(19) United States
(12) Patent Application Publication
(10) Pub. No.: US 2015/0079139 A1
(43) Pub. Date: Mar. 19, 2015

(54) HAIR PROCESSING AGENT COMPOSITION, AND HAIR PROCESSING AGENT COMPOSITION FOR DYEING HAIR . . . .

(71) Applicant: KIKUBOSHI CORPORATION, Tokyo (JP)

(72) Inventor: Miki TAKEHANA, Tokyo (JP)

(21) Appl. No.: 14/548,526

(22) Filed: Nov. 20, 2014

Related U.S. Application Data
(63) Continuation of application No. PCT/JP2013/080844, filed on Nov. 8, 2013.

(30) Foreign Application Priority Data
Nov. 8, 2012 (JP) 2012-246787

(51) Int. Cl. A61K 8/58; A61K 8/04; A61K 8/34; A61K 8/37; A61Q 5/06; A61Q 5/04; A61Q 5/02; A61Q 5/12; A61Q 5/00; A61K 8/02; A61Q 5/10

(52) U.S. Cl. CPC A61K 8/585; A61K 8/0245; A61K 8/0233; A61K 8/044; A61K 8/342; A61K 8/37; A61Q 5/10; A61Q 5/04; A61Q 5/02; A61Q 5/12; A61Q 5/002; A61Q 5/06
USPC 424/401; 424/70.1; 424/70.5; 424/70.51; 8/406; 510/119

(57) ABSTRACT

There is provided a hair procession agent composition including: 22% by mass or less of inorganic particles having an average particle size of primary particles of 1 nm to 1 μm, and an oil-soluble dispersant.

### CROSS REFERENCE TO RELATED APPLICATION

This application claims the benefit of and priority under 35 U.S.C. §119 to PCT Patent Application No. PCT/JP2013/080844, entitled “HAIR PROCESSING AGENT COMPOSITION, AND HAIR PROCESSING AGENT COMPOSITION FOR DYEING HAIR, FIRST AGENT FOR OXIDATION HAIR DYE, SECOND AGENT FOR OXIDATION HAIR DYE, HAIR PROCESSING AGENT COMPOSITION FOR SHAMPOOING, HAIR PROCESSING AGENT COMPOSITION FOR HAIR TREATMENT, HAIR PROCESSING AGENT COMPOSITION FOR PERMING, THIO-BASED HEATING TWO-BATH TYPE PERM SOLUTION FIRST AGENT, THIO-BASED HEATING TWO-BATH TYPE PERM SOLUTION SECOND AGENT, CYS-BASED HEATING TWO-BATH TYPE PERM SOLUTION FIRST AGENT AND CYS-BASED HEATING TWO-BATH TYPE PERM SOLUTION SECOND AGENT USING THE HAIR PROCESSING AGENT COMPOSITION,” filed on Nov. 8, 2013, which is hereby incorporated by reference in its entirety into this application.

### TECHNICAL FIELD

The present invention relates to a hair processing agent composition (a hair treating agent composition or a hair treatment agent composition). The present invention also relates, to a hair processing agent composition for dyeing hair, a first agent for oxidation hair dye, a second agent for oxidation hair dye, a hair processing agent composition for shampooing, a hair processing agent composition for hair treatment, a hair processing agent composition for perming, a thio-based (thio-type or thioglycolate-based) heating two-bath type (two-bath system or two-liquid system) perm solution first agent, a thio-based (thio-type or thioglycolate-based) heating two-bath type perm solution second agent, a cys-based (cys-type or cysteine based) heating two-bath type perm solution first agent and a cys-based (cys-type or cysteine-based) heating two-bath type perm solution second agent, each utilizing the aforementioned hair processing agent composition.

### BACKGROUND ART

In the case where hair is thin and soft by nature or by aging, or in the case where hair is damaged by perming or by hair coloring, the hair loses voluminousness (voluminous appearance), and bounce and resilience (strength, elasticity). Furthermore, when hair loses bounce and resilience, the hair loses shape retention strength, and styling to have a desired hair style becomes difficult.

Therefore, hair processing agents to impart voluminousness, bounce and resilience to hair have been proposed. Such a hair processing agent can be classified roughly into two types including a type of coating hair on the surface thereof (hereinbelow referred to as “coating type”) and a type of reinforcing hair from inside (hereinbelow referred to as “inside reinforcing type”).

As a hair processing agent of the coating type, there has been proposed, for example, a hair growth coloring dyeing agent containing coal particle (Patent Document 1). In addition, as another hair processing agent of a coating type, there has been proposed a hair styling agent composition containing hydrophobic silica particles and non-aqueous dispersion liquid (mainly ethanol) for dispersing the hydrophobic silica particles therein (Patent Document 2). By the hair growth coloring dyeing agent and the hair styling agent composition, hair is reinforced from outside by coating the surface of the hair with coal particles and hydrophobic silica particles to enable to impart voluminousness, bounce and resilience to the hair. However, in the case of using the aforementioned hair growth coloring dyeing agent and hair styling agent, composition, sticky feeling or stiff feeling, or both of those are imparted to the hair due to the coal particles and hydrophobic silica particles with which the surface of the hair is coated. In order to dissolve such discomfort feeling on the touch surface of hair, there has been proposed a foam head hair cosmetic material containing a silicone compound having a predetermined molecular structure (Patent Document 3). By coating hair with a film containing a silicone compound having a predetermined molecular structure, such as a film provided by the aforementioned foam head hair cosmetic material, it is possible to impart voluminousness, bounce and resilience to hair without causing sticky feeling and stiff feeling.

As a hair processing agent of the inside reinforcing type, there has been proposed a hair processing agent containing a univalent or divalent metal salt and a sericin hydrolysate (Patent Document 4). When using the hair processing agent containing sericin hydrolysate, a hydrolysate of silk protein sericin is infiltrated (penetrates) into hair, and the sericin hydrolysate is bonded to keratin of hair tissue inside the hair. Such bonding of the sericin hydrolysate and the keratin enables to reinforce the hair from inside and to impart voluminousness, bounce, and resilience to the hair. In addition, as another hair processing agent of the inside reinforcing type, there has been proposed a hair processing agent containing an alkoxysilane compound having a predetermined molecular structure (Patent Document 5). This hair processing agent containing an alkoxysilane compound enables to reinforce hair from inside and, therefore, impart voluminousness, bounce and resilience to hair, by producing a silanol compound from the alkoxysilane compound and infiltrating silanol compound into the hair to cause polymerization.

### RELATED TECHNICAL DOCUMENT

### Patent Document

Patent Document 1: JP 2009-73827 A.
Patent Document 2: JP 2011-246352 A
Patent Document 3: JP 2009-280527 A
Patent Document 4: JP 2009-132648 A
Patent Document 5: JP 2009-184978 A

### SUMMARY OF THE INVENTION

### Problem to be Solved by the Invention

However, in the aforementioned coating type hair processing agent, a coating material (coating film containing coal particles, hydrophobic, silica particles, a silicone compound, etc.) is peeled off when the hair is washed with a shampoo. As a result, the hair is put back in the original state where voluminousness, bounce, and resilience are lost. That is, it is difficult for the aforementioned coating type hair processing anent to maintain hair in a state having voluminousness, bounce, and resilience.

In contrast, the aforementioned inside reinforcing type hair processing agent is hardly influenced by washing hair with a shampoo or the like and can maintain hair in a state having voluminousness, bounce, and resilience. However, in the case of using the aforementioned inside reinforcing type hair processing agent since the reaction of combining a sericin hydrolysate with keratin and the polymerization reaction of a silanol compound are caused inside hair, the hair has to be maintained in a state of being suitable for these reactions. Therefore, in the case of using the aforementioned inside reinforcing type hair processing agent, it cannot be performed at the same time as another processing such as hair coloring (dyeing), hair washing (shampooing), hair treatment, or perming.

In view of the aforementioned problems, the present invention aims to provide a technique realizing the hair processing of imparting sustainable voluminousness, bounce and resilience to hair and being capable of conducting the hair processing simultaneously with dyeing, shampooing, hair treatment, or perming.

### Means for Solving the Problems

The present invention includes the following hair processing agent composition, and also includes the following hair processing agent composition for dyeing hair, first agent for oxidation hair dye, second agent for oxidation hair dye, hair processing agent composition for shampooing, hair processing agent composition on for hair treatment, hair processing agent composition for perming, thio-based heating two-bath type perm solution (perming liquid) first agent, thio-based (thioglycolate-containing) heating two-bath type perm solution second agent, cys-based (cysteine-containing) heating two-bath type perm solution first agent and cys-based heating two-bath type perm solution second agent, each containing the aforementioned hair processing agent composition.

[1] A hair processing agent composition including: 22% by mass or less of inorganic particles having an average Particle size of 1 nm to 1 nm at the time of primary particles, and an oil-soluble dispersant.

[2] The hair processing agent composition according to [1], including an inorganic particle dispersion where the inorganic particles, in a state of primary particles and/or secondary particles having an average particle size of 5 μm or less, are dispersed in the oil-soluble dispersant.

[3] The hair processing agent composition according to [2], including: a surfactant, and an aqueous solvent, wherein a first phase including the inorganic particle dispersion and a second phase including the aqueous solvent form an emulsion.

[4] The hair processing agent composition according to [1], including core-shell particles, each having a core portion including the primary particles of the inorganic particles or the secondary particles having an average particle size of 5 μm or less of the inorganic particles, and a shell layer including the oil-soluble dispersant and enclosing the core portion.

[5] The hair processing agent composition according to [4], including: a surfactant and an aqueous solvent, wherein the core-shell particles form a colloid dispersed in the aqueous solvent.

[6] The hair processing agent composition according to any one of [1] to [5], wherein the inorganic particles are silica particles.

[7] The hair processing agent composition according to any one of [1] to [6], including a moistening auxiliary.

[8] A hair processing agent composition for dyeing hair, including: the hair processing agent composition according to any one of [1] to [7], and a hair dye.

[9] The hair processing agent composition for dyeing hair according to [8], including: a first agent including an oxidation dye, and a second agent including the hair processing agent composition and hydrogen peroxide.

[10] The hair processing agent composition for dyeing according to [8], including: a first agent including the hair processing agent composition and an oxidation dye, and a second agent including hydrogen peroxide.

[11] A first agent of an oxidation hair dye including: the hair processing agent composition according to any one of [1] to [7], and an oxidation dye.

[12] A second agent of an oxidation hair dye including: she hair processing agent composition according to any one of [1] to [7], and hydrogen peroxide.

[13] A hair processing agent composition for shampooing, including: the hair processing agent composition according to any one of [1] to [7], and a shampoo agent.

[14] A hair processing agent composition for hair treatment, including: the hair processing agent composition according to any one of [1] to [7], and a hair treatment agent.

[15] The hair processing agent composition for hair treatment according to [14], wherein the hair treatment agent includes silicone.

[16] A hair processing agent composition for perming, including: the hair processing agent composition according to any one of [1] to [7], and a perming agent.

[17] The hair processing agent composition for perming according to [16], including: a thio-based heating two-bath type perm solution first agent including the hair processing agent composition, and a thio-based heating two-bath type perm solution second agent.

[18] The hair processing agent composition for perming according to [16], including: a thio-based heating two-bath type perm solution first agent, and a thio-based heating two-bath type perm solution second agent including the hair processing agent composition.

[19] The hair processing agent composition for perming according to [16], including: a cys-based heating two-bath type perm solution first agent including the hair processing agent composition, and a cys-based heating two-bath type perm solution second agent.

[20] The hair processing agent composition for perming according to [16], including: a cys-based heating two-bath type perm solution first agent, and a cys-based heating two-bath type perm solution second agent including the hair processing agent composition.

[21] A thio-based heating two-bath type perm solution first agent including: the hair processing agent composition according to any one of [1] to [7], and a reducing agent.

[22] A thio-based heating two-bath type perm solution second agent including: the hair processing agent composition according to any one of [1] to [7], and an oxidizing agent.

[23] A cys-based heating two-bath type perm solution first agent including: the hair processing agent composition according to any one of [1] to [7], and a reducing agent.

[24] A cys-based heating two-bath type perm solution second agent including: the hair processing agent composition according to any one of [1] to [7], and an oxidizing agent.

### Effect of the Invention

According to a hair processing agent composition of the present invention, inorganic particles are infiltrated into (let into) the inside of hair, specifically the gap between a cuticle edge portion exposed to the outside and an inside cuticle covered with the cuticle edge portion (concretely, adjacent cuticle on the tip side) in a scale structure on the surface of the hair formed of cuticles. In addition, according to a hair processing agent composition of the present invention, by stabilizing (fixing) the inorganic particles between the outside cuticle and the inside cuticle, hair can be reinforced and voluminousness, bounce, and resilience can be imparted to the hair. Since the inorganic particles stabilized inside the hair in such a manner are hardly released from the inside of the hair even by shampooing the hair, by a hair processing agent composition of the present invention, it is easy to maintain hair in a state of having voluminousness, and bounce and resilience.

Furthermore, with a hair processing agent composition of the present invention, hair can be reinforced by a simple system of introducing inorganic particles into the gap between a cuticle edge portion exposed to the outside and an inside cuticle covered with the edge portion. Therefore, the hair processing agent composition of the present invention is hardly influenced by the presence of a hair dye, a shampoo agent, a hair treatment agent or a perming agent, and at the same time, can impart voluminousness, and bounce and resilience to hair having a decreased influence on the hair dye and the like. That is, by using the hair processing agent composition of the present invention, a hair processing for imparting voluminousness, bounce and resilience to hair, and a processing of dyeing, shampooing, hair treatment or perming can be conducted at the same time.

### BRIEF DESCRIPTION OF THE DRAWINGS

FIG. 1 is a schematic explanatory view of an embodiment of a hair processing agent composition of the present invention.

FIG. 2 is a schematic explanatory view of another embodiment of a hair processing agent composition of the present invention.

FIG. 3 is a schematic explanatory view of an embodiment including a surfactant and an aqueous solvent among hair processing agent compositions of the present invention.

FIG. 4 is a schematic explanatory view of another embodiment including a surfactant and an aqueous solvent among hair processing agent compositions of the present invention.

FIG. 5A is an electron micrograph of a whole image of an unprocessed head hair.

FIG. 5B is an electron micrograph of an enlarged image of cuticles of an unprocessed, head hair.

FIG. 5C is an electron micrograph of an enlarged image of a cuticle edge portion of an unprocessed head hair.

FIG. 6A is an electron micrograph of a who image of a head hair after hair processing using the hair processing agent composition of Example 1.

FIG. 6B is an electron micrograph of an enlarged image of cuticles of a head hair after hair processing′ using the hair processing agent composition of Example 1.

FIG. 6C is an electron micrograph of an enlarged image of cuticle edge portion of a head hair after hair processing using the hair processing agent composition of Example 1.

FIG. 7A is an electron micrograph of a whole image of a head hair after hair processing using the hair processing agent composition of Example 2.

FIG. 7B is an electron micrograph of an enlarged image of cuticles of a head hair after hair processing using the hair processing agent composition of Example 2.

FIG. 7C is an electron micrograph of an enlarged image of a cuticle edge portion of a head hair after hair processing using the hair processing agent composition of Example 2.

FIG. 8A is an electron micrograph of a whole image of a head hair after hair processing using the hair processing agent composition of Comparative Example 1.

FIG. 8B is an electron micrograph of an enlarged image of cuticles of a head hair after hair processing using the hair processing agent composition of Comparative Example 1.

FIG. 8C is an electron micrograph of an enlarged image of a cuticle edge portion of a head hair after hair processing using the hair processing agent composition of Comparative Example 1.

FIG. 9A is an electron micrograph of a whole image of a head hair after dyeing hair using the hair processing agent composition for dyeing hair of Example 5.

FIG. 9B is an electron micrograph of an enlarged image of cuticles of a head hair after dyeing hair using the hair processing; agent composition for dyeing hair of Example 5.

FIG. 9C is an electron micrograph of an enlarged image of a cuticle edge portion of a head hair after dyeing hair using the hair processing agent composition for dyeing hair of Example 5.

FIG. 10A is an electron micrograph of a whole image of a head hair after shampooing hair and hair treatment of Usage Example 1 using the hair processing agent composition for shampooing of Example 25.

FIG. 10B is an electron micrograph of an enlarged image of cuticles of a head hair after shampooing hair and hair treatment of Usage Example 1 using the hair processing agent composition for shampooing of Example 25.

FIG. 10C is an electron micrograph of an enlarged image of a cuticle edge portion of a head hair after shampooing hair and hair treatment of Usage Example 1 using the hair processing agent composition for shampooing of Example 25.

FIG. 11A is an electron micrograph of a whole image of a head hair after shampooing hair and hair treatment of Usage Example 2 using the hair processing agent composition for shampooing of Example 25.

FIG. 11B is an electron micrograph of an enlarged image of cuticles of a head hair after shampooing hair and hair treatment of Usage Example 2 using the hair processing agent composition for shampooing of Example 25.

FIG. 11C is an electron micrograph of an enlarged image of a cuticle edge portion of a head hair after shampooing hair and hair treatment of Usage Example 2 using the hair processing agent composition for shampooing of Example 25.

FIG. 12A is an electron micrograph of a whole image of a head hair after shampooing hair and hair treatment of Usage Example 3 using the hair processing agent composition for hair treatment of Example 89.

FIG. 12B is an electron micrograph of an enlarged image of cuticles of a head hair after shampooing hair and hair treatment of Usage Example 3 using the hair processing agent composition for hair treatment of Example 89.

FIG. 12C is an electron micrograph of an enlarged image of a cuticle edge portion of a head hair after shampooing hair and hair treatment of Usage Example 3 using the hair processing agent composition for hair treatment of Example 89.

FIG. 13A is an electron micrograph of a whole image of a head hair after perming using the hair processing agent composition for perming of Example 125.

FIG. 13B is an electron micrograph of an enlarged image of cuticles of a head hair after perming using the hair processing agent composition for perming of Example 125.

FIG. 13C is an electron micrograph of an enlarged image of a cuticle edge portion of a head hair after perming using the hair processing agent composition for perming of Example 125.

FIG. 14A is an electron micrograph of a whole image of a head hair after perming using the hair processing agent composition for perming of Example 124.

FIG. 14B is an electron micrograph of an enlarged image of cuticles of a head hair after perming using the hair processing agent composition for perming of Example 124.

FIG. 14C is an electron micrograph of an enlarged image of a cuticle edge portion of a head hair after perming using the hair processing agent composition for perming of Example 124.

FIG. 15A is an electron micrograph of a whole image of a head hair after perming using the hair processing agent composition for perming of Example 125.

FIG. 15B is an electron micrograph of an enlarged image of cuticles of a head hair after perming using the hair processing agent composition for perming of Example 125.

FIG. 15C is an electron micrograph of an enlarged image of a cuticle edge portion of a head hair after perming using the hair processing agent composition for perming of Example 125.

FIG. 16A is an electron micrograph of a whole image of a head hair after perming using the hair processing agent composition for perming of Example 126.

FIG. 16B is an electron micrograph of an enlarged image of cuticles of a head hair after perming using the hair processing agent composition for perming of Example 126.

FIG. 16C is an electron micrograph of an enlarged image of a cuticle edge portion of a head hair after perming using the hair processing agent composition for perming of Example 126.

FIG. 17A is a photo of a hair bundle subjected to processing with the hair processing agent composition of Example 17.

FIG. 17B is a photo of a hair bundle subjected to processing with the hair processing agent composition of Example 18.

FIG. 17C is a photo of a hair bundle subjected to processing with the hair processing agent composition of Example 19.

FIG. 17D is a photo of a hair bundle subjected to processing with the hair processing agent composition of Example 20.

FIG. 17E is a photo of a hair bundle subjected to processing with the hair processing agent composition of Example 21.

FIG. 18A is a photo of a hair bundle subjected to processing with the hair processing agent composition for dyeing hair of Comparative Example 3.

FIG. 18B is a photo of a hair bundle subjected to processing with the hair processing agent composition for dyeing hair of Example 31.

FIG. 18C is a photo of a hair bundle subjected to processing with the hair processing agent composition for dyeing hair of Example 32.

FIG. 18D is a photo of a hair bundle subjected to processing with the hair processing agent composition for dyeing hair of Example 33.

FIG. 18E is a photo of a hair bundle subjected to a process with the hair processing agent composition for dyeing hair of Example 34.

FIG. 19A is a photo of the head of the subject A before processing.

FIG. 19B is a photo of the head of the subject A after processing with the hair processing agent composition for dyeing hair of Example 35.

FIG. 19C is a photo of the head of the subject B before processing.

FIG. 19D is a photo of the head of the subject B after processing with the hair processing agent composition for dyeing hair of Example 35.

FIG. 20A is a photo of an unprocessed hair bundle.

FIG. 20B is a photo of a hair bundle subjected to processing with the hair processing agent composition for dyeing hair of Comparative Example 4.

FIG. 20C is a photo of a hair bundle subjected to processing with the hair processing agent composition for dyeing hair of Example 36.

FIG. 20D is a photo of a hair bundle subjected to processing with the hair processing agent composition for dyeing hair of Example 37.

FIG. 20E is a photo of a hair bundle subjected to processing with the hair processing agent composition for dyeing hair of Example 38.

FIG. 20F is a photo of a hair bundle subjected to processing with the hair processing agent composition for dyeing hair of Example 39.

FIG. 20G is a photo of a hair bundle subjected to processing with the hair processing agent composition for dyeing hair of Example 40.

FIG. 20H is a photo of a hair bundle subjected to processing with the hair processing agent composition for dyeing hair of Example 41.

FIG. 20I is a photo of a hair bundle subjected to processing with the hair processing agent composition for dyeing hair of Example 42.

FIG. 20J is a photo of a hair bundle subjected to processing with the hair processing agent composition for dyeing hair of Example 43.

FIG. 21A is a photo of a hair bundle subjected to processing with the hair processing agent composition for dyeing hair of Comparative Example 5.

FIG. 21B is a photo of a hair bundle subjected to processing with the hair processing agent composition for dyeing hair of Example 44.

FIG. 21C is a photo of a hair bundle subjected to processing with the hair processing agent composition for dyeing hair of Example 45.

FIG. 21D is a photo of a hair bundle subjected to processing with the hair processing agent composition for dyeing hair of Example 46.

FIG. 21E is a photo of a hair bundle subjected to processing with the hair processing agent composition for dyeing hair of Example 47.

FIG. 21F is a photo of a hair bundle subjected to processing with the hair processing agent composition for dyeing hair of Example 48.

FIG. 21G is a photo of a hair bundle subjected to processing with the hair processing agent composition for dyeing hair of Example 49.

FIG. 21H is a photo of a hair bundle subjected to processing with the hair processing agent composition for dyeing hair of Example 50.

FIG. 21I is a photo of a hair bundle subjected to processing with the hair processing agent composition for dyeing hair of Example 51.

FIG. 22A is a photo of an unprocessed hair bundle.

FIG. 22B is a photo of a hair bundle subjected to processing with the hair processing agent composition for perming of Comparative Example 11.

FIG. 22C is a photo of a hair bundle subjected to processing with the hair processing agent composition for perming of Example 127.

FIG. 22D is a photo of a hair bundle subjected to processing with the hair processing agent composition for perming of Example 128.

FIG. 22E is a photo of a hair bundle subjected to processing with the hair processing agent composition for perming of Example 129.

FIG. 22F is a photo of a hair bundle subjected to processing with the hair processing agent composition for perming of Example 130.

FIG. 22G is a photo of a hair bundle subjected to processing with the hair processing agent composition for perming of Example 131.

FIG. 22H is a photo of a hair bundle subjected to processing with the hair processing agent composition for perming of Example 132.

FIG. 22I is a photo of a hair bundle subjected to processing with the hair processing agent composition for perming of Example 133.

FIG. 22J is a photo of a hair bundle subjected to processing with the hair processing agent composition for perming of Example 134.

FIG. 23A is a photo of a hair bundle subjected to processing with the hair processing agent composition for perming of Comparative Example 12.

FIG. 23B is a photo of a hair bundle subjected to processing with the hair processing agent composition for perming of Example 135.

FIG. 23C is a photo of a hair bundle subjected to processing with the hair processing agent composition for perming of Example 136.

FIG. 23D is a photo of a hair bundle subjected to processing with the hair processing agent composition for perming of Example 137.

FIG. 23E is a photo of a hair bundle subjected to processing with the hair processing agent composition for perming of Example 138.

FIG. 23F is a photo of a hair bundle subjected to processing with the hair processing agent composition for perming of Example 139.

FIG. 23G is a photo of a hair bundle subjected to processing with the hair processing agent composition for perming of Example 140.

FIG. 23H is a photo of a hair bundle subjected to processing with the hair processing agent composition for perming of Example 141.

FIG. 23I is a photo of a hair bundle subjected to processing with the hair processing agent composition for perming of Example 142.

FIG. 24 is a photo of a hair bundle subjected to processing with the hair processing agent composition for perming of Example 143.

FIG. 25 is a photo of a hair bundle subjected to processing with the hair processing agent composition for perming of Example 144.

FIG. 26A is a photo of an unprocessed hair bundle.

FIG. 26B is a photo of a hair bundle subjected to processing with the hair processing agent composition for perming of Comparative Example 13.

FIG. 26C is a photo of a hair bundle subjected to processing with the hair processing agent composition for perming of Example 145.

FIG. 26D is a photo of a hair bundle subjected to processing with the hair processing agent composition for perming of Example 146.

FIG. 26E is a photo of a hair bundle subjected to processing with the hair processing agent composition for perming of Example 147.

FIG. 26F is a photo of a hair bundle subjected to processing with the hair processing agent composition for perming of Example 148.

FIG. 26G is a photo of a hair bundle subjected to processing with the hair processing agent composition for perming of Example 149.

FIG. 26H is a photo of a hair bundle subjected to processing with the hair processing agent composition for perming of Example 150.

FIG. 26I is a photo of a hair bundle subjected to processing with the hair processing agent composition for perming of Example 151.

FIG. 26J is a photo of a hair bundle subjected to processing with the hair processing agent composition for perming of Example 152.

FIG. 27A is a photo of a hair bundle subjected to processing with the hair processing agent composition for perming of Comparative Example 14.

FIG. 27B is a photo of a hair bundle subjected to processing with the hair processing agent composition for perming of Example 153.

FIG. 27C is a photo of a hair bundle subjected to processing with the hair processing agent composition for perming of Example 154.

FIG. 27D is a photo of a hair bundle subjected to processing with the hair processing agent composition for perming of Example 155.

FIG. 27E is a photo of a hair bundle subjected to processing with the hair processing agent composition for perming of Example 156.

FIG. 27F is a photo of a hair bundle subjected to processing with the hair processing agent composition for perming of Example 157.

FIG. 27G is a photo of a hair bundle subjected to processing with the hair processing agent composition for perming of Example 158.

FIG. 27H is a photo of a hair bundle subjected to processing with the hair processing agent composition for perming of Example 159.

FIG. 27I is a photo of a hair bundle subjected to processing with the hair processing agent composition for perming of Example 160.

FIG. 28A is a photo of a hair bundle subjected to processing with the hair processing agent composition for perming of Comparative Example 15.

FIG. 28B is a photo of a hair bundle subjected to processing with the hair processing agent composition for perming of Example 161.

FIG. 28C is a photo of a hair bundle subjected to processing with the hair processing agent composition for perming of Example 162.

FIG. 28D is a photo of a hair bundle subjected to processing with the hair processing agent composition for perming of Example 163.

FIG. 28E is a photo of a hair bundle subjected to processing with the hair processing agent composition for perming of Example 164.

FIG. 28F is a photo of a hair bundle subjected to processing with the hair processing agent composition for perming of Example 165.

FIG. 28G is a photo of a hair bundle subjected to processing with the hair processing agent composition for perming of Example 166.

FIG. 28H is a photo of a hair bundle subjected to processing with the hair processing agent composition for perming of Example 167.

FIG. 28I is a photo of a hair bundle subjected to processing with the hair processing agent composition for perming of Example 168.

### MODE FOR CARRYING OUT THE INVENTION

Hereinbelow, embodiments of the present invention will be described referring to Drawings. The present invention is not limited to the following embodiments, and changes, modifications and improvements may be made as long as they do not deviate from the scope of the present invention.

1. Hair Processing Agent Composition:

A hair processing agent composition of the present invention includes 22% by mass or less of inorganic particles having an average particle size of primary particles of 1 nm to 1 μm and an oil-soluble dispersant.

Since the inorganic particles used for the present invention have an average particle size of 1 nm to 1 μm as primary particles, the inorganic particles have aggregating property, which is typical for so-called nanoparticles (characteristic that inorganic particles tend to aggregate together to easily form aggregates).

An oil-soluble dispersant used in the present invention has a function of inhibiting aggregation of inorganic particles. Since aggregation of inorganic particles is inhibited by a function of the oil-soluble dispersant, inorganic particles can easily be maintained in a state of primary particles and/or secondary particles having an average particle size of 5 μm or less.

The size of the primary particles of the inorganic particles having an average particle size of 1 nm to 1 μm or the secondary particles having an average particle size of 5 μm or less of the inorganic particles enable to allow the particles to enter the gap between a cuticle edge portion exposed to the outside and an inside cuticle covered with the cuticle edge portion (concretely, adjacent cuticles on the tip side) in hair. In the case where the average particle size of the secondary particles of the inorganic particles is 5 μm or less, when a hair processing agent composition of the present invention is applied to hair, the secondary particles of the inorganic particles tend to collapse (break) to form, from the secondary particles, primary particles having an average particle size of 1 μm or less and/or secondary particles having an average particle size of 1 μm or less. In this manner, in a hair processing agent composition of the present invention, the primary particles and/or the secondary particles of the inorganic particles having an average particle size of 1 μm or less are applied into hair. Therefore, in a hair processing agent composition of the present invention, the inorganic particles easily enter the gap between a cuticle edge portion exposed to the outside and a cuticle inside the cuticle edge portion.

In addition, an oil-soluble dispersant used in the present invention has an affinity for cuticles. Therefore, an oil-soluble dispersant used in the present invention plays a role of assisting introduction of the inorganic particles into the gap between a cuticle edge portion exposed to the outside and an adjacent cuticle covered with the cuticle edge portion and located on the tip side of the outside cuticle. In this manner, stabilization of the inorganic particles after the introduction into the gap between a cuticle edge portion exposed to the outside and an adjacent cuticle on the tip side of the outside cuticle, covered with the cuticle edge portion of the outside cuticle, enables to reinforce hair from the inside and impart voluminousness, and bounce and resilience to the hair.

In a hair processing agent composition of the present invention, the inorganic particles may aggregate to form aggregates as long as it is after the introduction of the inorganic particles into the gap between a cuticle edge portion exposed to the outside and an inside cuticle covered with the outside cuticle. In the case where the aggregates of inorganic particles are formed in a state of being sandwiched between an outside cuticle and an inside cuticle and stabilized (adhered) in this portion, the function of reinforcing the hair is further enhanced, and, as a result, it becomes possible to impart voluminousness, bounce and resilience to the hair. At this time, the aggregation force (cohesion force) of the inorganic particles is week to such extent that the aggregate is broken by applying an external force such as bending the hair. Therefore, according to the present invention, even in the case where aggregates of the inorganic particles are stabilized in a state of being sandwiched between an outside cuticle and an inside cuticle, hair can be maintained in a moderately soft state without being solidified too firmly.

FIG. 1 is a schematic explanatory view of an embodiment of a hair processing agent composition of the present invention. A hair processing agent composition of the present invention may include an inorganic particle dispersion 1 as shown in the figure. In the present embodiment, the inorganic particle dispersion 1 is a dispersion in which inorganic particles 2 are dispersed in an oil-soluble dispersant 5 in a state of primary particles 3 and/or secondary particles 4 having an average particle size of 5 μm or less.

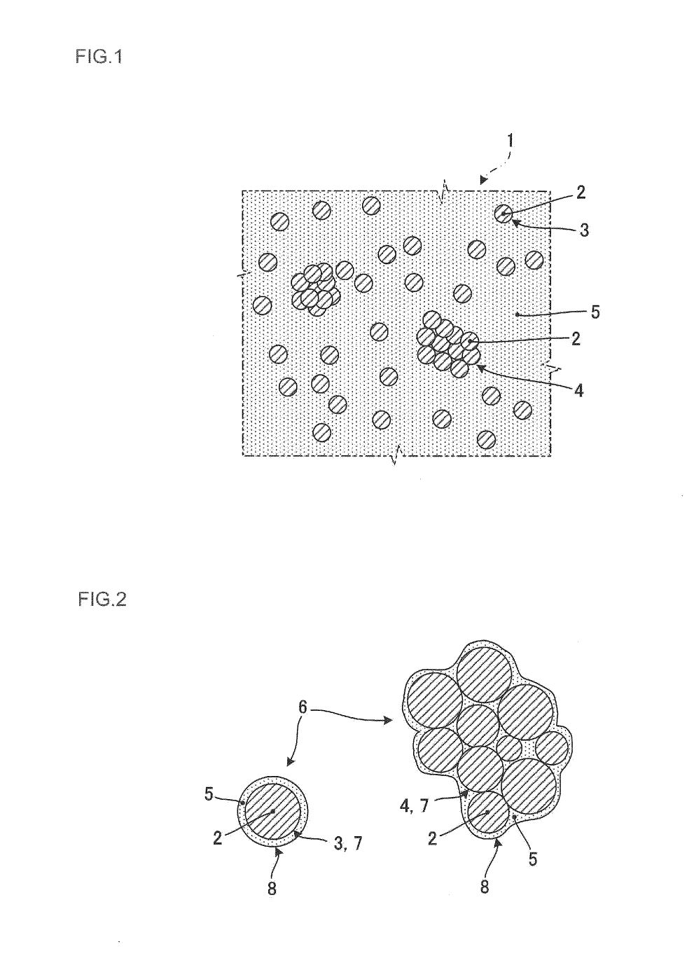

FIG. 2 is a schematic explanatory view of another embodiment of a hair processing agent composition of the present invention. A hair processing agent composition of the present invention may contain core-shell particles 6 as shown in the figure. The core-shell particles 6 have a core portion 7 including the primary particles 3 of the inorganic particles 2 or the secondary particles 4 having an average particle size of 5 μm or less of the inorganic particles 2, and a shell layer 8 including the oil-soluble dispersant 5 and enclosing the core portion 7.

In addition, a hair processing agent composition of the present invention may further include a surfactant and an aqueous solvent.

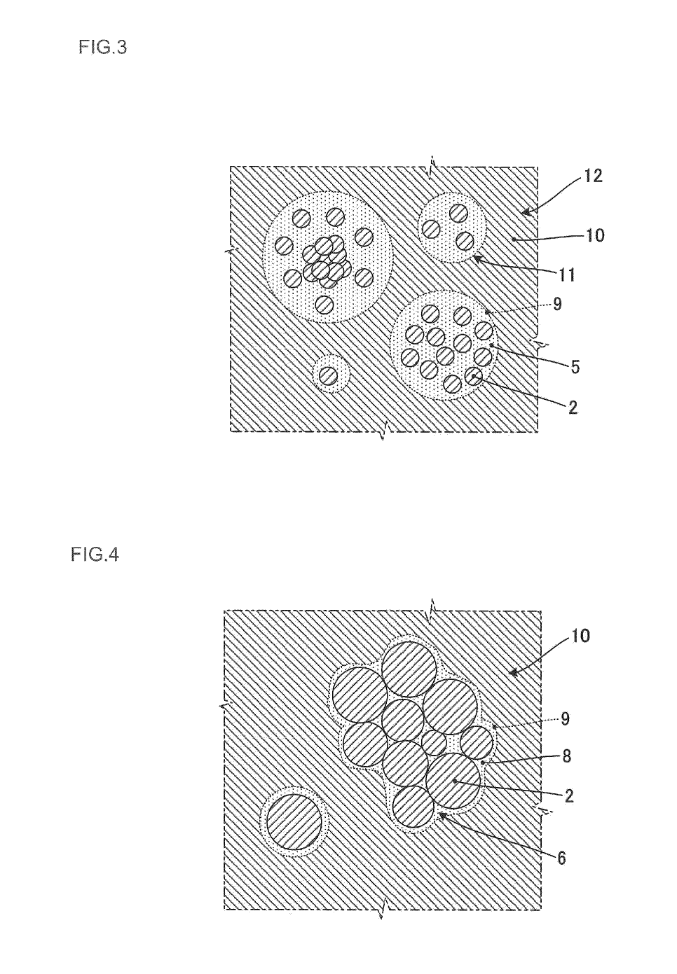

FIG. 3 is a schematic explanatory view of an embodiment including a surfactant and an aqueous solvent among hair processing agent compositions of the present invention. As shown in the figure, in a hair processing agent composition of the present invention, in the case of including a surfactant 9 and an aqueous solvent 10, a phase (first phase 11) including the inorganic particle dispersion 1 and a phase (second phase 12) including the aqueous solvent 10 may form an emulsion. In this case, the surfactant 9 is interposed between the first phase 11 and the second phase 12 to play a role of maintaining the emulsion stably.

In the case where the aforementioned emulsion is formed in a hair processing agent composition of the present invention, the continuous phase may be the first phase 11 or the second phase 12. However, the continuous phase is preferably the second phase 12. In the case where the aforementioned emulsion is formed in a hair processing agent composition of the present invention, the second phase 12 as the continuous phase enables to easily maintain the inorganic particles 2 in the state of the primary particles 3 and/or the secondary particles 4 having an average particle size of 5 μm or less. As a result, it becomes easy to introduce inorganic particles 2 into the gap between a cuticle edge portion exposed to the outside and an inside cuticle covered with the cuticle edge portion.

As a method for preparing the aforementioned embodiment where the first phase 11 and the second phase 12 form an emulsion, for example, an inorganic particle dispersion 1 (see, e.g., FIG. 1) is prepared in advance, and an aqueous solvent 10 and a surfactant 9 may be added to it, followed by stirring them. By such a method, an emulsion can be formed.

Alternatively, as another method for preparing the embodiment where the first phase 11 and the second phase 12 form an emulsion, there is the following method. First, an oil-soluble dispersant 5, an aqueous solvent 10, and a surfactant 9 are mixed together, and the mixture is stirred to form an emulsion (preemulsion) of a phase including the oil-soluble dispersant 5 and a phase including the aqueous solvent 10 (second phase 12) in advance. Next, the inorganic particles 2 are added to the preemulsion, and stirring is performed strongly to mix the inorganic particles 2 into the phase including the oil-soluble dispersant 5. By this method, the phase including the inorganic particle dispersion 1 (first phase 11) can be formed.

This method for forming the preemulsion is particularly suitable for the case where the inorganic particles 2 are silica particles. When the inorganic particles 2 are silica particles, it is sometimes difficult to disperse silica particles in the oil-soluble dispersant 5. When it is difficult to disperse such silica particles, a preemulsion as described above may be formed in advance, and silica particles may be added thereto, followed by strongly stirring. By this method, it becomes possible to allow the silica particles to be adsorbed by the oil-soluble dispersant 5 to compulsorily introduce the silica particles into the oil-soluble dispersant 5 and disperse them.

FIG. 4 is a schematic explanatory view of another embodiment including a surfactant and an aqueous solvent among hair processing agent compositions of the present invention. As shown in the figure, in a hair processing agent composition of the present invention, in the case of containing the surfactant 9 and the aqueous solvent 10, there may be formed a colloid (colloidal dispersion) where the core-shell particles 6 are dispersed in the aqueous solvent 10. In this case, the surfactant 9 is interposed between the core-shell particles 6 and the aqueous solvent 10 to play a role of stably maintaining the colloid.

In the case where the surfactant 9 and the aqueous solvent 10 are contained in a hair processing agent composition of the present invention, even if water-soluble components (e.g., moistening auxiliaries described later, and hydrogen peroxide in the case of hair processing agent composition for dyeing hair) are added, the function of the water-soluble components can sufficiently be exhibited.

It is preferable that an hair processing agent composition of the present invention contains a moistening auxiliary (moistening coagent, humectant). The moistening auxiliary which can be used for the present invention swells hair and plays a role of easing the introduction of inorganic particles into the gap between a cuticle edge portion exposed to the outside and an inside cuticle covered with the cuticle edge portion. Therefore, in a hair processing agent composition of the present invention, in the case of containing a moistening auxiliary, the inorganic particles can be introduced and stabilized more securely into the gap between a cuticle edge portion exposed to the outside and an inside cuticle covered with the exposed cuticle edge portion. Therefore, in the case where a hair processing agent composition of the present invention contains a moistening auxiliary, bounce and resilience can be imparted to hair more securely.

In addition, as long as the hair processing agent composition of the present invention has the aforementioned characteristic, any substance can be used as the inorganic particle, the oil-soluble dispersant, the surfactant, the aqueous solvent, or the moistening auxiliary.

In addition, a hair processing agent composition of the present invention described so far can be used as a hair processing agent composition for dyeing hair by mixing it into a hair dye or using it together with a hair dye. In addition, a hair processing agent composition of the present invention can be used as a hair processing agent composition for shampooing by mixing it into a shampoo agent or using it together with a shampoo agent. A hair processing agent composition of the present invention can be used as a hair processing agent composition for hair treatment by mixing it into a hair treatment agent or using it together with a hair treatment agent. Further, a hair processing agent composition of the present invention can be used as a hair processing agent composition for perming by mixing it into a perming agent or using it together with a perming agent (described later in detail).

Hereinbelow, each of the components which is contained (which may be contained) in a hair processing agent composition of the present invention is described in more detail.

(Inorganic Particles)

As the inorganic particles used for the present invention, there can be mentioned silica particles (hydrophobic silica particles, hydrophilic silica particles), calcium phosphate particles, calcium carbonate particles, titanium oxide particles, carbon particles, talc particles, mica particles, and sodium silicate particles.

The average particle size of the primary particles of the inorganic particles used for the present invention is preferably 1 nm to 900 nm from the viewpoint of easing introduction of the inorganic particles into the gap between an cuticle edge portion exposed to the outside and an inside cuticle covered with the cuticle edge portion. Further, the average particle size of the primary particles of the inorganic particles used for the present invention is more preferably 1 nm to 500 nm, and particularly preferably 1 nm to 50 nm.

In addition, in the present invention, in the case where the secondary particles of the inorganic particles are dispersed in the oil-soluble dispersant, the average particle size of the secondary particles of the inorganic particles is preferably 3 μm or less. In the case where the average particle size of the secondary particles of the inorganic particles is 3 μm or less, it becomes easy to introduce the inorganic particles into the gap between a cuticle edge portion exposed to the outside and an adjacent cuticle on the tip side of the outside cuticle and covered with the outside cuticle edge portion. Further, in the present invention, in the case where the secondary particles of the inorganic particles are dispersed in the oil-soluble dispersant, the average particle size of the secondary particles of the inorganic particles is more preferably 2 μm or less, particularly preferably 50 nm to 1.5 μm, most preferably 50 nm to 1.0 μm.

The average particle size in the present specification is, in the case where the average particle size of 1 μm or less is measured, a measured value (modal diameter) measured by a dynamic scattering method (photon correlation method) and obtained from the number-size distribution in a particle diameter analysis method “CONTIN method”. In addition, the average particle size in the present specification is a value (median diameter) measured by a laser diffraction method, in the case where the average particle size of above 1 μm is measured.

In a hair processing agent composition of the present invention, the inorganic particle content is preferably 0.1 to 22% by mass because the inorganic particles are easily dispersed in the oil-soluble dispersant as the primary particles having an average particle size of 1 nm to 1 μm or the secondary particles having an average particle size of 5 μm or less. Further, in a hair processing agent composition of the present invention, the inorganic particle content is more preferably 0.5 to 12% by mass, furthermore preferably 1.0 to 8.0% by mass, particularly preferably 2.0 to 7.0% by mass, most preferably 2.0 to 5.0% by mass.

Furthermore, as the inorganic particles used in the present invention, silica particles (hydrophobic silica particles, hydrophilic silica particles) are preferable. In particular, hydrophobic silica particles are more preferable as the inorganic particles used in the present invention because of having a characteristic of easy stabilization between a cuticle edge portion exposed to the outside and an inside cuticle covered with the cuticle edge portion (high affinity for cuticles).

In a hair processing agent composition of the present invention, in the case where the inorganic particles are hydrophobic silica particles, the hydrophobic silica particles can be once stabilized between a cuticle edge portion exposed to the outside and an inside cuticle covered with the cuticle edge portion of the outside cuticle. When hydrophobic silica particles are once stabilized between cuticles in such a manner as above, the hydrophobic silica particles are hardly released from the gap between the overlapped cuticles to the outside of the hair, due to the high affinity of the hydrophobic silica particles and cuticles. Therefore, in a hair processing agent composition of the present invention, in the case where the inorganic particles are hydrophobic silica particles, it is easy to maintain the hair in the state of having voluminousness, bounce and resilience.

Further, in a hair processing agent composition of the present invention, in the case where the inorganic particles are hydrophobic silica particles, the hydrophobic silica particles and aggregates of the hydrophobic silica particles can be stabilized so that they span the gap between a cuticle edge portion exposed to the outside and an inside cuticle covered with the cuticle edge portion. Stabilizing the hydrophobic silica particles and aggregates of the hydrophobic silica particles in such a configuration enables to make a condition where the overlapped cuticles are crosslinked, in combination with the high affinity between the hydrophobic silica particles and cuticles. In the case where such a crosslinked state is formed, it is possible to prevent cuticles from flipping up or to repair flipping of the cuticles. Therefore, in the case where the inorganic particles are hydrophobic silica particles in a hair processing agent composition of the present invention, it becomes possible to prevent hair from being damaged and to repair damage to hair, and it becomes possible to inhibit split ends from being caused.

In a hair processing agent composition of the present invention, particularly in the case where the inorganic particles include hydrophobic silica particles, the hydrophobic silica particle content (content with respect to the entire hair processing agent composition as 100% by mass) is preferably 0.5 to 22% by mass. In the case where the hydrophobic silica particle content is 0.5 to 22% by mass, the primary particles having an average particle size of 1 nm to 1 μm and the secondary particles having an average particle size of 5 μm or less can easily be dispersed in the oil-soluble dispersant. Furthermore, in the case where the hydrophobic silica particle content is 0.5 to 22% by mass, it can be inhibited that the hydrophobic silica particles are aggregated before entering the gap between a cuticle edge portion exposed to the outside and an inside cuticle covered with the cuticle edge portion. In a hair processing agent composition of the present invention, in the case where the inorganic particles include hydrophobic silica particles, the hydrophobic silica particle content is preferably 1.0 to 12% by mass, particularly preferably 1.5 to 8.0% by mass, most preferably 1.5 to 6.0% by mass.

As hydrophobic silica particles usable for the present invention, there can be mentioned, for example, particles obtained by subjecting the hydroxyl group (—OH) on the surfaces of the silica particles to a treatment of dimethyl silylation, trimethyl silylation, or the like. Here, regarding the characteristics of the hydrophobic silica particles, a treating method such as dimethyl silylation will be explained with examples. However, the explanation does not mean to limit the hydrophobic silica particles used in the present invention to the particles obtained by the treating method shown as an example.

As marketed hydrophobic silica particles usable for the present invention, there can be mentioned, for example, AEROSIL-R972, AEROSIL-R974, AEROSIL-R104, AEROSIL-R106, AEROSIL-R202, AEROSIL-R805, AEROSIL-R812, AEROSIL-R812S, AEROSIL-R816, AEROSIL-R7200, AEROSIL-R8200, AEROSIL-R9200, AEROSIL-R711, AEROSIL-R972Pharma (produced by Nippon Aerosil Co., Ltd.), VM-2270 Aerogel Fine Particles (produced by Dow Corning Corp.), HDK H2000, HDK H15, HDK H18, HDK H20, and HDK H30 (produced by Wacker Asahikasei Silicone, Co., Ltd.).

As marketed hydrophilic silica particles usable for the present invention, there can be mentioned, for example, AEROSIL-90, AEROSIL-130, AEROSIL-150, AEROSIL-200, AEROSIL-255, AEROSIL-300, AEROSIL-380, AEROSIL-OX50, AEROSIL-TT600, AEROSIL-200SP, AEROSIL-300SP, AEROSIL-200Pharma, AEROSIL-300Pharma, AEROSIL-300/30, AEROSIL-300Pharma (produced by Nippon Aerosil Co., Ltd.), HDK N20, HDK T30 (produced by Wacker Asahikasei Silicone, Co., Ltd.), Sylysia350, and Sylisia445 (produced by Fuji Silysia Chemical Ltd.)

<Oil-Soluble Dispersant>

As oil-soluble dispersants usable for the present invention, there can be mentioned polyols and higher alcohols.

Polyols usable as the oil-soluble dispersant in the present invention, there can be mentioned, for example, glycerin, concentrated glycerin, pentylene glycol, butylene glycol, 1,3-butylene glycol, ethylene glycol, diethylene glycol, propylene glycol, polyethylene glycol, and ethoxy diglycol.

As higher alcohols usable as the oil-soluble dispersant for the present invention, there can be mentioned, for example, isostearic acid, isostearyl alcohol, oleyl alcohol, hexyldecanol, octyldodecanol, decyl alcohol, cetanol, cetostearyl alcohol, stearyl alcohol, behenyl alcohol, hydrogenated rapeseed oil alcohol, myristyl alcohol, arachyl alcohol, lauryl alcohol, chimyl alcohol, cholesterol, lanoline alcohol, hexyl decanol, 2-octyldecanol, selachyl alcohol (monooleyl glyceryl ether), batyl alcohol (glyceryl monostearyl ether), phytosterol (phytosterin), hydrogenated lanoline alcohol, capryl alcohol, linolyl alcohol, POE stearyl ether, and POE cetyl ether.

Further, as oil-soluble dispersants usable for the present invention, there can be mentioned various oils including vegetable oils such as hardened rapeseed oil alcohol and isostearic acid hardened (hydrogenated) castor oil; esters such as tri(caprylic acid/capric acid) glyceryl and cetyl 2-ethyl hexanoate; lower alcohols such as iropropanol; hydrocarbons such as liquid paraffin and liquid isoparaffin; waxes such as beeswax; fatty acids such as lauric acid, myristic acid, and laurylic acid; and silicone oils such as cyclopentasiloxane, dimethicone, and cyclomethicone.

Because a stable colloid or emulsion can be formed in the case where the hair processing agent composition contains an aqueous solvent and a surfactant, it is preferable that the oil-soluble dispersant used for the present invention contains at least one selected from the group consisting of cetanol, lanoline alcohol, cetearyl alcohol, liquid paraffin, stearyl alcohol, polyethylene glycol, isopropanol, cetanol, propylene glycol, highly polymerized methylpolysiloxane, butylene glycol, glycerin, lipophilic glyceryl stearate, vaseline, phytosterol, octyl decanol, myristyl alcohol, decamethylcyclopentasiloxane, liquid isoparaffin, cyclotetrasiloxane, castor oil, isostearyl alcohol, methylpolysiloxane, isostearic acid hydrogeneted castor oil isostearyl alcohol, methylpolysiloxane, octamethylcyclotetrasiloxane, highly polymerized polyethylene glycol, aminoethyl-aminopropylmethylsiloxane-dimethylsiloxane copolymer, highly polymerized dimethicone-1, 2-propanol, hardened oil, cholesterol, vegetable squalane, palm kernel oil, palm oil, glycol stearate, dimethicone, microcrystalline wax, beeswax, oleyl alcohol, phytosteryl oleate, DPG, behenyl alcohol, glyceril tricaprinate, glyceril tricaprylate, and methyl polysiloxane. Furthermore, because a stable colloid or emulsion can be obtained in the state that the inorganic particles (particularly, hydrophobic silica particles are suitable) are dispersed well in the oil-soluble dispersant, it is more preferable that the oil-soluble dispersant used for the present invention includes at least one selected from the group consisting of cetanol, lanoline alcohol, cetearyl alcohol, liquid paraffin, stearyl alcohol, polyethylene glycol, isopropanol, propylene glycol, highly polymerized methylpolysiloxane, butylene glycol, glycerin, lipophilic glyceryl stearate, vaseline, phytosterol, octyldecanol, myristyl alcohol, decamethylcyclopentasiloxane, liquid isoparaffin, and hardened rapeseed oil alcohol.

In a hair processing agent composition of the present invention, in the case where an emulsion containing an inorganic particle dispersion is formed (e.g., the case of the state shown in FIG. 3) or the case where a colloid in which core-shell particles are dispersed in an aqueous solvent is formed (e.g., the case of the state shown in FIG. 4), the content of the oil-soluble dispersant (content with respect to the entire hair processing agent composition as 100% by mass) is preferably 0.55 to 50% by mass. When the content of the oil-soluble dispersant is 0.55 to 50% by mass, the emulsion or colloid can be stabilized, and this can easily introduce inorganic particles between a cuticle edge portion exposed to the outside and an inside cuticle covered with the cuticle edge portion. Furthermore, in a hair processing agent composition of the present invention, the oil-soluble dispersant content (content with respect to the entire hair processing agent composition as 100% by mass) is more preferably 1 to 10% by mass, particularly preferably 1 to 5% by mass, most preferably 3 to 5% by mass.

In a hair processing agent composition of the present invention, it is preferable that the oil-soluble dispersant contains cetanol and lanoline alcohol because a ball bearing effect is generated by mixing the oil-soluble dispersant, water and the surfactant, and, as a result, a stable emulsion can easily be obtained. Such ball bearing effect enables to easily induce micelles enclosing inorganic particles (particularly, hydrophobic silica particles are suitable) therein.

In a hair processing agent composition of the present invention, in the case where the hair processing agent composition contains an aqueous solvent and a surfactant and where the oil-soluble dispersant includes cetanol and lanoline alcohol, the content of cetanol is preferably 0.5 to 5% by mass or less from the viewpoint of obtaining a stable emulsion. In particular, in a hair processing agent composition of the present invention, it is preferable that the oil-soluble dispersant includes cetanol and lanoline alcohol, and that the cetanol content is 0.5 to 5% by mass, and that the lanoline alcohol content is 0.05 to 0.5% by mass [the aforementioned contents (% by mass) are with respect to the entire hair processing agent composition as 100% by mass].

<Surfactant>

As surfactants usable for the present invention, there can be mentioned anionic surfactants, cationic surfactants, non-ionic surfactants, and amphoteric surfactants.

As the anionic surfactants usable as a surfactant in the present invention, there can be mentioned, for example, fatty acid sodium (NS soap, SS-40N, KS soap, OS soap, FR-14, FR-25: Kao Corporation), sodium lauryl sulfate (Emal: Kao Corporation), sodium higher alcohol sulfate (Emal: Kao Corporation), triethanolamine lauryl sulfate (Emal: Kao Corporation), ammonium lauryl sulfate (Latemul: Kao Corporation), dodecylbenzene sulfonate (Neopelex: Kao Corporation), sodium dodecylbenzene sulfonate (Neopelex: Kao Corporation), dodecylbenzene sulfonate (Neopelex: Kao Corporation), sodium alkylnaphthalene sulfonate (Pelex: Kao Corporation), sodium dialkyl sulfosuccinate (Pelex: Kao Corporation), sodium alkyldiphenyl ether disulfonate (Pelex: Kao Corporation), sodium alkane sulfonate (Latemul: Kao Corporation), polyoxyalkylene alkenyl ether ammonium sulfate (Latemul: Kao Corporation), polyoxyethylene alkyl ether potassium phosphate (Electrostripper F: Kao Corporation), dipottasium alkenylsuccinate (Latemul ASK: Kao Corporation), monoalkyl sulfate, alkylpolyoxyethylene sulfate (Emal: Kao Corporation), alkylbenzene sulfonate, monoalkyl phosphate, sodium alpha-sulfofatty acid ester, sodium alpha-olefin sulfonate, sodium alkyl sulfonate, straight chain sodium alkylbenzene sulfonate, sodium alkyl ether sulfate ester, sodium polyoxylauryl ether sulfate (TEXAPONN25 Maker: BASF Japan Ltd.), sodium polyoxyethylene alkyl ether sulfate (Emal, Latemul: Kao Corporation), sodium polyoxyethylene alkyl ether sulfate (Levenol, Latemul), triethanol amine polyoxyethylene alkyl ether sulfate (Emal: Kao Corporation), sodium octanoate, sodium decanoate, sodium laurate, sodium myristate, sodium palmitate, sodium stearate, PFOA (C₇F₁₅COOH), perfluorononanoic acid, N-lauroylsarcosine sodium, sodium cocoylglutamate, alpha-sulfo fatty acid methyl ester salt, sodium 1-hexanesulfonate, sodium 1-octanesulfonate, sodium 1-decanesulfonate, sodium 1-dodecanesulfonate, perfluorobutane sulfonic acid, straight chain sodium alkylbenzene sulfonate, sodium toluene sulfonate, sodium cumene sulfonate, sodium octylbenzene sulfonate, DBS, sodium naphthalene sulfonate, disodium naphthalene disulphonate, trisodium naphthalene trisulfonate, sodium butylnaphthalene sulfonate, PFOS (C₈F₁₇SO₃H), sodium myristyl sulfate, sodium laureth sulfate, sodium polyoxyethylene alkyl phenol sulfonate, ammonium lauryl sulfate, lauryl phosphoric acid, sodium lauryl phosphate, and potassium lauryl phosphate.

As cationic surfactants usable as a surfactant in the present invention, there can be mentioned, for example, alkylamine salts such as coconut amine acetate (Acetamin 24: Kao Corporation) and stearyl amine acetate (Acetamin 86: Kao Corporation); and quarternary ammonium salts such as lauryltrimethylammonium chloride (Quartamin 24P: Kao Corporation), stearyltrimethylammonium chloride (Quartamin 86P Conc: Kao Corporation), cetyltrimethylammonium chloride (Quartamin 60W: Kao Corporation), stearyltrimethylammonium chloride (Quartamin 86W: Kao Corporation), alkylbenzyldimethylammonium chloride (Quartamin D86P: Kao Corporation), and distearyldimethylammonium chloride (Sanisol C, Sanisol B-50: Kao Corporation).

As non-ionic surfactants usable as a surfactant in the present invention, there can be mentioned, for example, polyoxyethylene lauryl ether (Emulgen: Kao Corporation), polyoxyethylene cetyl ether (Emulgen: Kao Corporation), polyoxyethylene stearyl ether (Emulgen: Kao Corporation), polyoxyethylene oleyl ether (Emulgen: Kao Corporation), polyoxyethylene alkyl ether (Emulgen: Kao Corporation), polyoxymyristyl ether (Emulgen: Kao Corporation), polyoxyethylene octyldodecyl ether (Emulgen: Kao Corporation), polyoxyethylene alkylenealkyl ether (Emulgen: Kao Corporation), polyoxyethylene distyrenated phenyl ether (Emulgen: Kao Corporation), polyoxyalkylenealkenyl ether (Latemul, Kao Corporation), sorbitan fatty acid ester (Rheodol, Emasol: Kao Corporation), sorbitan monolaurate (Rheodol: Kao Corporation), sorbitan monopalmitate (Rheodol: Kao Corporation), sorbitan monostearate (Rheodol: Kao Corporation), sorbitan distearate (Rheodol: Kao Corporation), sorbitan tristearate (Rheodol: Kao Corporation), sorbitan monooleate (Rheodol: Kao Corporation), sorbitan trioleate (Rheodol: Kao Corporation), sorbitan monolaurate (Rheodol Super SP-L10: Kao Corporation), sorbitan monostearate (Rheodol: Kao Corporation), sorbitan monooleate (Rheodol: Kao Corporation), sorbitan sesquioleate (Rheodol: Kao Corporation), polyoxysorbitan fatty acid ester (Rheodol: Kao Corporation), polyoxyethylene sorbitan monolaurate (Rheodol: Kao Corporation), polyoxyethylene sorbitan monopalmitate (Rheodol: Kao Corporation), polyoxysorbitan monostearate (Rheodol: Kao Corporation), polyoxysorbitan tristearate (Rheodol: Kao Corporation), polyoxyethylene sorbitan monooleate (Rheodol: Kao Corporation), polyoxyethylene sorbitan trioleate (Rheodol: Kao Corporation), polyoxyethylene sorbitan triisostearate (Rheodol: Kao Corporation), polyoxyethylene sorbitan monolaurate (Rheodol Super: Kao Corporation), tetraoleic acid polyoxyethylene sorbitol (Rheodol: Kao Corporation), glycerin fatty acid ester (Rheodol: Kao Corporation), glycerol monostearate (Rheodol: Kao Corporation), glycerol monooleate (Rheodol: Kao Corporation), self-emulsifying glycerol monostearate (Rheodol: Kao Corporation), polyoxyethylene fatty acid ester (Emanon: Kao Corporation), polyethyleneglycol monolaurate (Rheodol: Kao Corporation), polyethyleneglycol monostearate (Rheodol: Kao Corporation), polyethyleneglycol distearate (Rheodol: Kao Corporation), polyethyleneglycol monooleate (Rheodol: Kao Corporation), polyoxyethylene hardened castor oil (Emanon: Kao Corporation), polyoxyethylene alkyl amine (Amiet: Kao Corporation), alkyl alkanolamide (Aminon: Kao Corporation), sucrose fatty acid ester, alkyl polyglycoside, fatty acid diethanol amide, alkyl monoglyceryl ether, polyoxyethylene sorbitan fatty acid ester, fatty acid alkanol amide, polyoxyethylene alkyl ether, polyoxyethylene alkyl phenyl ether, glycerin laurate, glycerin monostearate, polyoxyethylene alkyl ether, pentaethylene glycol monododecyl ether, octaethylene glycol monododecyl ether, polyoxyethylene alkyl phenyl ether, nonoxynol, nonoxynol-9, polyoxyethylene polyoxypropylene glycol, polyoxyethylene hexytane fatty acid ester, sorbitane fatty acid ester polyethylene glycol, diethanol amide laurate, diethanol amide oleate, diethanol amide stearate, cocamide DEA, octyl glucoside, decyl glucoside, and lauryl glucoside.

As amphoteric surfactants usable as a surfactant in the present invention, there can be mentioned, for example, alkylamino fatty acid sodium, alkyl betaine (Amphitol: Kao Corporation), alkyl amine oxide (Amphitol: Kao Corporation), alkyl dimethyl amine oxide, alkyl carboxy betaine, alkyl amine oxide, lauryl betaine (Amphitol: Kao Corporation), stearyl betaine (Amphitol: Kao Corporation), 2-alkyl-N-carboxymethyl-N-hydroxyethyl isodazolinium betaine (Amphitol: Kao Corporation), lauryldiemthyl amine oxide (Amphitol: Kao Corporation), lauryldimethylaminoacetic acid betaine, stearyldimethylaminoacetic acid betaine, dodecylaminomethyldimethylsulfopropyl betaine, octadecylaminomethyldimethylsulfopropyl betaine, cocamidopropyl betaine, cocamidopropylhydroxy sultaine, 2-alkyl-N-carboxymethyl-N-hydroxyethylimidazolinium betaine, sodium lauroyl glutamate, potassium lauroyl glutamate, lauroylmethyl-β-alanine, lauryldimethylamine N-oxide, and oleyldiemthylamine N-oxide.

In the case of using a surfactant in a hair processing agent composition of the present invention, it is possible to employ only one kind or a combination of two or more kinds of the aforementioned various surfactants. In addition, the selection from the aforementioned various surfactants may appropriately be performed in consideration of compatibility with the oil-soluble dispersant and aqueous solvent.

<Aqueous Solvent>

As aqueous solvents usable in the present invention, there can be mentioned, for example, water or a liquid containing water and additives (e.g., pH adjuster, preservative, chelator).

As pH adjusters usable for the aqueous solvent in the present invention, there can be mentioned, for example, triethanolamine, disodium hydrogenphosphate, phosphoric acid, citric acid, lactic acid, malic acid, and glycolic acid.

In a hair processing agent composition of the present invention, the pH of the aqueous solvent is preferably 3 to 10, more preferably 5 to 10. In the case where the pH of the aqueous solvent is 3 to 10 in a hair processing agent composition of the present invention, hair is swelled to facilitate the introduction of inorganic particles into the gap between a cuticle edge portion exposed to the outside and an inside cuticle covered with the cuticle edge portion. Furthermore, in a hair processing agent composition of the present invention, the pH of the aqueous solvent is more preferably 5 to 10, most preferably 5.5 to 9.8.

As preservatives usable for the aqueous solvent in the present invention, there can be mentioned, for example, phosphoric acid, salicylic acid, parabens, and phenoxyethanol.

As chelators usable for the aqueous solvent in the present invention, there can be mentioned, for example, hydroxyethanediphosphonic acid, edetates, and citric acid.

<Moistening Auxiliary>

As moistening auxiliaries usable in the present invention, there can be mentioned, for example, alkaline agent, silicone oil, mineral oil, vegetable oil, and animal oil. Some of the moistening auxiliaries described below can function as oil-soluble dispersants. In such a case, it is recognized that the oil-soluble dispersants can play a role as a moistening auxiliary.

As alkaline agents usable as the moistening auxiliary in the present invention, there can be mentioned, for example, strong ammonia water, monoethanol amine, triethanol amine, ammonium hydrogen carbonate, and ammonium chloride.

As silicone oils usable as the moistening auxiliary in the present invention, there can be mentioned, for example, methylphenyl silicone oil, cyclic dimethyl silicone oil (acrylates/tridecyl acrylate/triethoxysilylpropyl methacrylate/dimethicone methacrylate) copolymer, methicone, hydrogen dimethicone, triethoxysilylethylpolydimethylsiloxyethylhexyl dimethicone, triethyoxycaprylylsilane, dimethicone, cyclopentasiloxane, diphenyldimethicone, diphenylsiloxyphenyltrimethicone, diemthiconol, amodimethicone, aminopropyldimethicone, and simethicone.

As mineral oils usable as the moistening auxiliary in the present invention, there can be mentioned, for example, light liquid paraffin, liquid paraffin, vaseline, ceresin, solid paraffin, and microcrystalline wax.

As vegetable oils usable as the moistening auxiliary in the present invention, there can be mentioned, for example, ricinus (castor oil), tung oil, jatropha curcus oil (jatropha oil), stillingia oil, linseed oil (flaxseed oil, linseed oil), shortening, salad oil, refined oil, corn oil, soybean oil, sesame oil (sesame oil), rapeseed oil (canola oil), safflower oil (safflower oil), sunflower oil, rice oil, rice bran oil, camellia oil, palm oil, perm kernel oil, coconut oil (copra oil), cottonseed oil, hempseed oil (hemp oil), grape seed oil, poppy seed oil (poppy seed oil), mustard oil, wheat germ oil, evening primrose oil, beefsteak plant oil, perilla oil, kaya oil, persic oil (apricot kernel oil), akebia oil (akebia seed oil), camellia sinensis leaf oil (sasanqua oil), tee oil, walnut oil, white birch oil, chaulmoogra oil, croton oil, nutmeg oil, olive oil, peanut oil, almond oil, avocado oil, hazelnut oil, grape seed oil, laurel oil (laurel fruit oil), macadamia nut oil, argan oil, pumpkin seed oil, pecan nut oil, pistachio oil, jojoba oil, cacao butter, borage oil (borage seed oil), shea butter, babassu oil, baobab oil, melon seed oil, mango butter, sal butter, kokum butter, meadowfoam seed oil, coffee bean oil, marula nut oil, broccoli seed oil, peach kernel oil, cherry kernel oil, cranberry seed oil, pomegranate seed oil, kukui nut oil, neem oil (margosa oil), kiwi fruit seed oil, rose hip oil, sea buckthorn (sea buckthorn seed oil), mongongo oil, lindera praecox oil, zinc oxide oil, Mitsuda-yu oil (perilla oil in which lead oxide is added), Japan tallow, candelilla wax, carnauba wax, and essence (essential oil).

As animal oils used as the moistening auxiliary in the present invention, there can be mentioned, for example, lard, fett (beef tallow), bone fat, bone oil, fish oil, chicken oil, duck oil, goose oil, chrysalis oil, crab oil, horse oil, lanolin (wool grease), schmaltz, butter, emu oil, Soyu (milk oil or butter oil), ghee, horse oil, whale oil, dolphin oil, mink oil (squalane), egg-yolk oil, liver oil, shark oil, neat's-foot oil, and beeswax.

2. Hair Processing Agent for Dyeing Hair:

A hair processing agent composition for dyeing hair of the present invention contains the aforementioned hair processing agent composition (“hair processing agent composition of the present invention” in the section of “1. Hair processing agent composition”) and a hair dye. By a hair processing agent composition for dyeing hair of the present invention, hair processing for imparting voluminousness, bounce and resilience, and hair dyeing can be performed at the same time.

As hair dye agents usable for a hair processing agent composition for dyeing hair of the present invention, there can generally be mentioned alkaline oxidation hair dye, acidic oxidation hair dye, non-oxidizing hair dye, acidic dye (hair manicure), HC dye, basic dye, and dye called henna. The oxidation hair dye is a two agent type having a first agent including an oxidation dye and a second agent including hydrogen peroxide. The oxidation hair dye contains a component facilitating infiltration of the oxidation dye into the inside of hair. In an embodiment of a hair processing agent composition of the present invention, the function of the component contained in the hair dye, which facilitates the infiltration, can be utilized also for the phenomenon where the inorganic particles are introduced into the gap between overlapped cuticles. The function of facilitating the infiltration by the component contained in the hair dye enables to introduce the inorganic particles into hair more securely and, as a result, to impart voluminousness, bounce and resilience more securely. In addition, as a hair dye usable here, there may be employed pigments (e.g., Black No. 401) capable of dyeing hair by the use of a component of a hair processing agent composition of the present invention.

When a hair processing agent composition for dyeing hair of the present invention is applied to hair, by a function due to the thixotropic property of inorganic particles, an irritating component (component causing a stimulus or a pain when the component soaks into a scalp) contained in the hair dye hardly influences pain points of the scalp. As a result, a stimulus or a pain is hardly caused in the scalp.

In the hair processing agent composition for dyeing hair of the present invention, it is preferable that it includes a first agent including an oxidation dye, and a second agent including the hair processing agent composition and hydrogen peroxide. In the case of including a first agent including an oxidation dye and a second agent including the hair processing agent composition and hydrogen peroxide, the hair swelling function by the hydrogen peroxide can be used efficiently, and a stimulus and a pain in the scalp can be inhibited more securely.

In addition, the first agent of an oxidation hair dye (the first agent of an oxidation hair dye of the present invention) may include the aforementioned hair processing agent composition (“hair processing agent composition of the present invention” in the section of the “1. Hair processing agent composition”) and oxidation dye. By appropriately combining the separately prepared second agent of the oxidation hair dye with the first agent of an oxidation hair dye, hair can be dyed.

Alternatively, the second agent may be the second agent of an oxidation hair dye (the second agent of an oxidation hair dye of the present invention) including the aforementioned hair processing agent composition (“hair processing agent composition of the present invention” in the section of the “1. Hair processing agent composition”) and hydrogen peroxide. By appropriately combining the separately prepared first agent of an oxidation hair dye with the second agent of an oxidation hair dye, hair can be dyed.

Furthermore, in the hair processing agent composition for dyeing hair of this configuration, it is preferable that the hair processing agent composition includes an inorganic particle dispersion where the inorganic particles are dispersed in the oil-soluble dispersant in a state of primary particles and/or secondary particles having an average particle size of 5 μm or less, a surfactant, and an aqueous solvent, and that a first phase including the inorganic particle dispersion and a second phase including the aqueous solvent form an emulsion (note that also the aforementioned case of the first agent of an oxidation hair dye of the present invention and the second agent of an oxidation hair dye of the present invention may fall under the above). Thus, in an embodiment where the hair processing agent composition forms emulsion, the function of swelling hair by hydrogen peroxide can appear sufficiently because hydrogen peroxide is very compatible with the aqueous solvent contained in the second phase of the hair processing agent composition. As a result, the inorganic particles can be introduced well into the gap between a cuticle edge portion exposed to the outside and an inside cuticle covered with the cuticle edge portion.

Alternatively, in the hair processing agent composition for dyeing hair of this configuration, it is preferable that the hair processing agent composition includes core-shell particles, a surfactant and an aqueous solvent, and that the core-shell particles are dispersed in the aqueous solvent to form a colloid (note that also the aforementioned case of the first agent of an oxidation hair dye of the present invention and the second agent of an oxidation hair dye of the present invention may fall under the above). It is added that the core-shell particles described herein have a core portion including the primary particles of the inorganic particles or the secondary particles having an average particle size of 5 μm or less of the inorganic particles and a shell layer including the oil-soluble dispersant and enclosing the core portion. In an embodiment where a hair processing agent composition forms a colloid in such a manner as above, the function of swelling hair by hydrogen peroxide can appear sufficiently because hydrogen peroxide is very compatible with the aqueous solvent in the hair processing agent composition. As a result, the inorganic particles can be introduced well into the gap between a cuticle edge portion exposed to the outside and an inside cuticle covered with the cuticle edge portion.

As a concrete embodiment in the aforementioned case of the two agent type in a hair processing agent composition for dyeing hair of the present invention, there can be mentioned an embodiment wherein a second agent is obtained by mixing a hair processing agent composition (“hair processing agent composition of the present invention” in the section of “1. Hair processing agent composition”) with hydrogen peroxide, and a first agent (including oxidation dye) of a marketed oxidation hair dye is combined with the second agent. In this case, other components such as a pH adjuster and a chelator may be added to the second agent. In this manner, in a hair processing agent composition for dyeing hair of the present invention, very simple handling of combining a hair processing agent composition with a marketed oxidation hair dye, can dye hair and impart voluminousness, bounce and resilience at the same time.

In addition, a hair processing agent composition for dyeing hair of the present invention may contain the first agent including the hair processing agent composition (“hair processing agent composition of the present invention” in the section of “1. Hair processing agent composition”) and an oxidation dye, and the second agent including hydrogen peroxide. As a concrete embodiment in the case of such a two agent type, there can be mentioned an embodiment wherein a first agent is obtained by mixing a hair processing agent composition (“hair processing agent composition of the present invention” in the section of “1. Hair processing agent composition”) with an oxidation dye, and a marketed second agent (containing hydrogen peroxide) is combined with the first agent.

3. Hair Processing Agent Composition for Shampooing:

A hair processing agent composition for shampooing of the present invention contains the aforementioned hair processing agent composition (“hair processing agent composition of the present invention” in the section of “1. Hair processing agent composition”) and a shampoo agent. In a hair processing agent composition for shampooing of the present invention, hair processing for imparting voluminousness, bounce and resilience and hair washing (shampooing) can be performed at the same time. As shampoo agents usable here, there can be mentioned a soap-based shampoo, a higher alcohol-based shampoo, and an amino acid-based shampoo.

As an embodiment of a hair processing agent composition for shampooing of the present invention, there may be mentioned a mixture of a hair processing agent composition (“hair processing agent composition of the present invention” in the section of “1. Hair processing agent composition”) and a marketed shampoo agent. In this manner, in a hair processing agent composition for shampooing of the present invention, very simple handling of combining a hair processing agent composition with a marketed shampoo agent makes it possible to wash hair (shampoo) and impart voluminousness, bounce and resilience at the same time.

In a hair processing agent composition for shampooing of the present invention, it is preferable that the hair processing agent composition includes an inorganic particle dispersion, a surfactant and an aqueous solvent, and that a first phase including the inorganic particle dispersion and a second phase including the aqueous solvent form an emulsion. It is added that the inorganic particle dispersion described here is a dispersion where the inorganic particles are dispersed in the oil-soluble dispersant in a state of primary particles and/or secondary particles having an average particle size of 5 μm or less. In an embodiment where a hair processing agent composition forms an emulsion in such a manner as above, the inorganic particles can be introduced well into the gap between a cuticle edge portion exposed to the outside and an inside cuticle covered with the cuticle edge portion.

Alternatively, in a hair processing agent composition for shampooing of the present invention, it is preferable that the hair processing agent composition includes core-shell particles, a surfactant and an aqueous solvent, and that the core-shell particles are dispersed in the aqueous solvent to form a colloid. It is added that the core-shell particles described here mean particles having a core portion including the primary particles of the inorganic particles or the secondary particles having an average particle size of 5 μm or less of the inorganic particles, and a shell layer including the oil-soluble dispersant and enclosing the core portion. In an embodiment where a hair processing agent composition forms a colloid in such a manner as above, the inorganic particles can be introduced well into the gap between a cuticle edge portion exposed to the outside and an inside cuticle covered with the cuticle edge portion.

4. Hair Processing Agent Composition for Hair Treatment:

A hair processing agent composition for hair treatment of the present invention includes the aforementioned hair processing agent composition (“hair processing agent composition of the present invention” in the section of “1. Hair processing agent composition”) and a hair treatment agent. In a hair processing agent composition for hair treatment of the present invention, hair processing for imparting voluminousness, bounce and resilience and hair treatment can be performed at the same time. As treatment agents usable here, there can be mentioned agents categorized as a non-cation treatment or a cation treatment.

Hair treatment agents usable for a hair processing agent composition for hair treatment of the present invention include agents called hair rinse, hair conditioner, or hair pack.

As one embodiment of a hair processing agent composition for hair treatment of the present invention, there can be mentioned a mixture of the hair processing agent composition (“hair processing agent composition of the present invention” in the section of “1. Hair processing agent composition”) and a marketed hair treatment agent. In this manner, in a hair processing agent composition for hair treatment of the present invention, very simple handling of combining a hair processing agent composition with a marketed hair treatment agent can give hair a treatment and impart voluminousness, bounce and resilience at the same time.

In a hair processing agent composition for hair treatment of the present invention, it is preferable that the hair processing agent composition includes an inorganic particle dispersion, a surfactant and an aqueous solvent, and that a first phase including the inorganic particle dispersion and a second phase including the aqueous solvent form an emulsion. It is added that the inorganic particle dispersion described here is a body where the inorganic particles are dispersed in the oil-soluble dispersant in a state of primary particles and/or secondary particles having an average particle size of 5 μm or less. In an embodiment where a hair processing agent composition forms an emulsion in such a manner as above, the inorganic particles can be introduced well into the gap between a cuticle edge portion exposed to the outside and an inside cuticle covered with the cuticle edge portion.

Alternatively, in a hair processing agent composition for hair treatment of the present invention, it is preferable that the hair processing agent composition includes core-shell particles, a surfactant and an aqueous solvent, and that the core-shell particles are dispersed in the aqueous solvent to form a colloid. It is added that the core-shell particles described here mean particles having a core portion including the primary particles of the inorganic particles or the secondary particles having an average particle size of 5 μm or less of the inorganic particles, and a shell layer including the oil-soluble dispersant and enclosing the core portion. In an embodiment where a hair processing agent composition forms a colloid in such a manner as above, the inorganic particles can be introduced well into the gap between a cuticle edge portion exposed to the outside and an inside cuticle covered with the cuticle edge portion.

In a hair processing agent composition for hair treatment of the present invention, from the viewpoint of imparting bounce and resilience and allowing hair to rise in a better degree, it is preferable that the hair treatment agent contains silicone.

5. Hair Processing Agent Composition for Perming:

A hair processing agent composition for perming of the present invention includes the aforementioned hair processing agent composition (“hair processing agent composition of the present invention” in the section of “1. Hair processing agent composition”) and a perming agent. In a hair processing agent composition for perming of the present invention, hair processing for imparting voluminousness, bounce and resilience, and perming can be performed at the same time.

A hair processing agent composition for perming of the present invention may include a thio-based heating two-bath type (thio-based exothermic two-step type) perm solution first agent including the hair processing agent composition and a thio-based heating two-bath type perm solution second agent, or may include a thio-based heating two-bath type perm solution first agent and a thio-based heating two-bath type perm solution second agent including the hair processing agent composition.

Alternatively, a hair processing agent composition for perming of the present invention may include a cys-based heating two-bath type (cys-based exothermic two-step type) perm solution first agent including the hair processing agent composition and a cys-based heating two-bath type perm solution second agent, or may include a cys-based heating two-bath type perm solution first agent and a cys-based heating two-bath type perm solution second agent including the hair processing agent composition.

As perming agents (permanent wave agents) used for the hair processing agent composition for perming of the present invention, there can be mentioned, besides the aforementioned thio-based heating two-bath type perming agent (thio-based exothermic two-step permanent wave agent) and cys-based heating two-bath type perming agent (cys-based exothermic two-step permanent wave agent), a thio-based cold two-bath type perming agent, a cys-based cold two-bath type perming agent, a thio-based cold one bath type perming agent, a thio-based first agent prior preparation heat generation two-bath type perming agent (a thio-based first agent exothermic two-step type permanent wave agent for preparation at time of use), a thio-based cold two-bath type hair straightening agent, a thio-based heating two-bath type hair straightening agent, a heating two-bath type hair straightening agent using an iron for thio-based high temperature hair dressing, a sulfite-based perming agent, a cysteamine-based perming agent, a butyrolactonetiol-based perming agent [e.g., Spiera (registered trademark) (Showa Denko K.K.)], and tioglycerin-based perming agent, etc.

As an embodiment of a hair processing agent composition for perming of the present invention, there can be mentioned a mixture of a hair processing agent composition (“hair processing agent composition of the present invention” in the section of “1. Hair processing agent composition”) with a marketed perming agent. Thus, in a hair processing agent composition for perming of the present invention, very simple handling of combining a hair processing agent composition with a marketed perming agent can make it possible to perm and impart voluminousness, bounce and resilience at the same time.

In addition, there may be employed a thio-based heating two-bath type perm solution first agent (thio-based heating two-bath type perm solution first agent of the present invention) including the aforementioned hair processing agent composition (“hair processing agent composition of the present invention” in the section of “1. Hair processing agent composition”) and a reducing agent. By appropriately combining the thio-based heating two-bath type perm solution first agent with the thio-based heating two-bath type perm solution second agent prepared separately, perming becomes possible.

As reducing agents usable for the thio-based heating two-bath type perm solution first agent of the present invention, there can be mentioned thioglycolate salt, ammonium thioglycolate, and monoethanolamine thioglycolate. In a thio-based heating two-bath type perm solution first agent of the present invention, one kind out of the substances described above as reducing agents may be used, or two or more kinds may be used in combination.

In a thio-based heating two-bath type perm solution first agent of the present invention, there may appropriately be contained alkaline agents (e.g. strong ammonia water, monoethanol amine, and ammonium hydrogen carbonate), a chelate agent, dithioglycolic acid, a conditioning agent (a conditioner), and an aroma chemical (a perfume material).

Alternatively, there may be employed a thio-based heating two-bath type perm solution second agent (thio-based heating two-bath type perm solution second agent of the present invention) including the aforementioned hair processing agent composition (“hair processing agent composition of the present invention” in the section of “1. Hair processing agent composition”) and an oxidizing agent. By appropriately combining the thio-based heating two-bath type perm solution second agent with the thio-based heating two-bath type perm solution first agent prepared separately, perming becomes possible.

As oxidizing agents usable for the thio-based heating two-bath type perm solution second agent of the present invention, there can be mentioned hydrogen peroxide, bromate salt, potassium bromate, and sodium bromate.

In a thio-based heating two-bath type perm solution second agent of the present invention, a chelate agent, pH adjuster (e.g., phosphate, sodium phosphate, potassium phosphate), a conditioning agent, an aroma chemical, and the like may appropriately be contained.

Furthermore, there may be employed a cys-based heating two-bath type perm solution first agent (cys-based heating two-bath type perm solution first agent of the present invention) including the aforementioned hair processing agent composition (“hair processing agent composition of the present invention” in the section of “1. Hair processing agent composition”) and a reducing agent. By appropriately combining the cys-based heating two-bath type perm solution first agent with the cys-based heating two-bath type perm solution second agent separately prepared, perming becomes possible.

As reducing agents usable for a cys-based heating two-bath type perm solution first agent of the present invention, there can be mentioned cysteine (e.g., DL-cysteine hydrochloride, DL-cystine hydrochloride, L-cysteine, L-cysteine hydrochloride, acetylcysteine). In a cys-based heating two-bath type perm solution first agent, one kind out of the various kinds of cysteines described above as reducing agents may be used, or two or more kinds may be used in combination.

In a cys-based heating two-bath type perm solution first agent of the present invention, there may appropriately be contained alkaline agents (e.g. strong ammonia water, monoethanol amine, ammonium hydrogen carbonate), a chelate agent, dithioglycolate, a conditioning agent, and an aroma chemical.

Alternatively, there may be employed a cys-based heating two-bath type perm solution second agent (cys-based heating two-bath type perm solution second agent of the present invention) including the aforementioned hair processing agent composition (“hair processing agent composition of the present invention” in the section of “1. Hair processing agent composition”) and an oxidizing agent. By appropriately combining the cys-based heating two-bath type perm solution second agent with the cys-based heating two-bath type perm solution first agent separately prepared, perming becomes possible.

As oxidizing agents usable for the cys-based heating two-bath type perm solution second agent of the present invention, there can be mentioned hydrogen peroxide, bromate salt, potassium bromate, and sodium bromate.

In a cys-based heating two-bath type perm solution second agent of the present invention, a chelate agent, pH adjuster (e.g., phosphate, sodium phosphate, and potassium phosphate), a conditioning agent, an aroma chemical, and the like may appropriately be contained.

In a hair processing agent composition for perming of the present invention, it is preferable that the hair processing agent composition includes an inorganic particle dispersion, a surfactant and an aqueous solvent, and that a first phase including the inorganic particle dispersion and a second phase including the aqueous solvent form an emulsion (note that also the aforementioned cases of the thio-based heating two-bath type perm solution first agent of the present invention, the thio-based heating two-bath type perm solution second agent of the present invention, the cys-based heating two-bath type perm solution first agent of the present invention, and the cys-based heating two-bath type perm solution second agent of the present invention may fall under the above). It is added that the inorganic particle dispersion described here is a body where the inorganic particles are dispersed in the oil-soluble dispersant in a state of primary particles and/or secondary particles having an average particle size of 5 μm or less. In an embodiment where a hair processing agent composition thus forms an emulsion, inorganic particles can be introduced well into the gap between a cuticle edge portion exposed to the outside and an inside cuticle covered with the cuticle edge portion.

Alternatively, in a hair processing agent composition for perming of the present invention, it is preferable that the hair processing agent composition includes core-shell particles, a surfactant and an aqueous solvent, and that the core-shell particles are dispersed in the aqueous solvent to form a colloid (note that also the aforementioned cases of the thio-based heating two-bath type perm solution first agent of the present invention, the thio-based heating two-bath type perm solution second agent of the present invention, the cys-based heating two-bath type perm solution first agent of the present invention, and the cys-based heating two-bath type perm solution second agent of the present invention may fall under the above). It is added that the core-shell particles described here mean particles having a core portion including the primary particles of the inorganic particles or the secondary particles having an average particle size of 5 μm or less of the inorganic particles, and a shell layer including the oil-soluble dispersant and enclosing the core portion. In an embodiment where a hair processing agent composition thus forms a colloid, inorganic particles can be introduced well into the gap between a cuticle edge portion exposed to the outside and an inside cuticle covered with the cuticle edge portion.

### EXAMPLES

Hereinbelow, the present invention will be described in more detail on the basis of Examples. However, the present invention is not limited to these Examples.

### (1) Materials

The following materials were used in the following Examples and Comparative Examples.

(1-1) Silica Particles:

AEROSIL-R812 (AEROSIL R812, Evonic, average particle size of 7 nm)

(1-2) Oil-Soluble Dispersant:

Cetanol (Kokyu Alcohol Kogyo Co., Ltd.)

Lanolin alcohol (SUPER HARTOLAN, Croda Japan K.K.)

Cetostearyl alcohol (Kokyu Alcohol Kogyo Co., Ltd.)

Stearyl alcohol (Kalcol 8688, Kao Corporation)

Stearic acid (NAA-173K, NOF Corporation)

Hardened rapeseed oil alcohol (alcohol No. 20-B, Kokyu Alcohol Kogyo Co., Ltd.)

(1-3) Surfactant:

Sodium polyoxyethylene lauryl ether sulfate (TEXAPONN25, BASF Japan Ltd.)

Sodium laurylglycol acetate (Beaulight SHAA, Sanyo Kasei Industry Co., Ltd.

(1-4) pH Adjuster:

Triethanolamine (trade name: High Purity Triethanolamine (TEA-99), produced by Mitsui Chemicals, Inc.)

Disodium hydrogenphosphate (special grade disodium hydrogenphosphate (anhydrous), Kishida Chemical Co., Ltd.)

Phosphoric acid (special grade, phosphoric acid, Kishida Chemical Co., Ltd.)

(1-5) Chelate agent:

Hydroxyethane diphosphonic acid (TURPINAL SL, Cognis Japan)

(1-6) Preservative:

Salicylic acid (salicylic acid, Yoshitomi Pharmaceutical Industries, Ltd.)

(1-7) Hair Dye Agent:

### (1-7-1) Oxidation Hair Dye:

Trade name: Wella Koleston Perfect J, 6 brightness•black (6/0) (P&G) (first agent)

Trade name: In-fit Illumiant (International Toiletries Co., Ltd.) (first agent)

Trade name: Welloxon Perfect (P&G) (second agent)

### (1-7-2) Acidic Dye: Wella Color Fresh Black 0 (P&G)

### (1-7-3) HC Basic Dye:

Trade name: “BC Hair Color Treatment BL” (color treatment for Funwari-finishing (airy finishing)) (Medicos Corporation)

(1-8) Shampoo agent:

(1-8-1) Soap-based shampoo:

Trade name: COCO NATURA Shampoo (Kikuboshi Corporation)

### (1-8-2) Higher Alcohol-Based Shampoo:

Trade name: Pantene Extra Damage Hair Shampoo (P&G)

### (1-8-3) Amino Acid-Based Shampoo

Trade name: Lasana Shampoo (Yamasaki Co., Ltd.)

(1-9) Hair Treatment Agent:

### (1-9-1) Non-Cation Treatment:

Trade name: COCO NATURA Treatment (Kikuboshi Corporation)

(1-9-2) Cation treatment:

Trade name: Pantene Pro-V Extra Damage Care Treatment (P&G)

(1-10) Perming Agent:

### (1-10-1) Thio-Based Heating Two-Bath Type Perming Agent:

Trade name: Cream S. DESIGN LIPID (first agent, second agent) (Lebel Cosmetics)

### (1-10-2) Cys-Based Heating Two-Bath Type Perming Agent:

Trade name: CREAM CYS (first agent, second agent) (Lebel Cosmetics)

### (1-10-3) Thio-Based Cold Two-Bath Type Perming Agent:

Trade name: Stealth TA Normal-Light Damage Hair (first agent, second agent) (Number Three Inc.)

### (1-10-4) Cys-Based Cold Two-Bath Type Perming Agent:

Trade name: Stealth CYS CY damage level 4 to 5 (Number Three Inc.)

### (1-10-5) Cysteamine-Based Perming Agent:

Trade name: FELADYCA tocosme Cream L I CREAM LINER <hair treatment> (first agent) (Number Three Inc.)

Trade name: FELADYCA tocosme Second Cream II SECOND CREAM <hair treatment> (second agent) (Number Three Inc.)

### (1-10-6) Butyrolactonetiol-Based Perming Agent:

Trade name: Safety Wave Labo SP Curl 1 Spiera R-mixed two-bath type curling agent cosmetic product, exclusive to professionals (first agent) (Nousfit Corporation)

Trade name: Safety Wave Labo SP Fix 2 Safety exclusive to Wave Labo SP Cosmetic product, exclusive to professionals for fixing curl (second agent) (Nousfit Corporation)

(1-10-7) Sulfite-based perming agent:

Trade name: Creator Cosme 37<set lotion> (first agent) (Shiseido Professional)

Trade name: Creator Cosme AT <after treatment> (second agent) (Shiseido Professional)

(1-11) Others:

Ethanol (Japan Alcohol Corporation, general alcohol (alcohol for industrial usage) 95 degree synthesis)

Glycerin (Product Name: Concentrated Glycerin for Cosmetics, Kao Corporation)

Propylene glycol (PG) [Product Name: Propylene Glycol (Japanese Pharmacopoeia), Adeka Corporation]

Decamethylcyclopentasiloxane (TSF405, Momentive P•W•J)

28% aqueous ammonia (reagent 1st grade, Taisei Kakou Co., Ltd.)

50% ammonium thioglycolate solution (Sasaki Chemical Co., Ltd.)

100% monoethanolamine (Sasaki Chemical Co., Ltd.)

(used by diluting it to be 80% by purified water upon preparation)

L-cysteine (Tomo Chemical Co., Ltd.)

sodium bromate (Osakasasaki Chemical Co., Ltd.)

monosodium phosphate (Taihei Chemical Industrial Co., Ltd.)

dipotassium phosphate (Taihei Chemical Industrial Co., Ltd.)

### (2) Evaluation Items and Evaluation Criteria

In the following Examples and Comparative Examples, performance evaluations were given on the basis of the following evaluation items and evaluation criteria.

(2-1) Bounce and Resilience:

Excellent (Excel.): Bounce and resilience of hair can be felt to a considerable extent by touch.

Good: Bounce and resilience of hair can be felt to some extent.

Fair: Bounce and resilience of hair can be felt to a small extent.

Failed: Almost no bounce and resilience of hair can be felt.

(2-2) Rise:

Excellent (Excel.): Hair rises from the roots to a considerable extent.

Good: Hair rises from the roots to some extent.

Fair: Though hair rises from the roots, it lies down in a moment.

Failed: Hair does not rise from the roots.

(2-3) Stiffness:

Excellent (Excel.): Almost no stiffness of hair is felt.

Good: Not much stiffness of hair is felt.

Fair: Though some stiffness of hair is felt, it is not enough to bother.

Failed: Stiffness of hair bothers.

(2-4) Sustainability:

Excellent (Excel.): Bounce and resilience are sustained for 10 days or more.

Good: Bounce and resilience are sustained for 5 to 9 days.

Fair: Bounce and resilience are sustained for 3 to 4 days.

Failed: Bounce and resilience are sustained only for at most 2 days.

(2-5) Voluminousness:

Excellent (Excel.): Much volume is given.

Good: Volume is given.

Fair: Slight volume is given.

Failed: No volume is given.

### (3) Hair Processing Agent Composition

### Examples 1 to 25, Comparative Examples 1 and 2

The hair processing agent compositions of Examples 1 to 25 and Comparative Examples 1 and 2 were prepared by blending the aforementioned materials to have compositions shown in Table 1 (Examples 1 and 2), Table 2 (Comparative Example 1), Table 3 (Examples 3 to 16 and Comparative Example 2), and Table 4 (Examples 17 to 25) [The content (mass %) of each material in Tables 1 to 4 is a value based on the entire amount of the hair processing agent composition (the entire amount of the hair processing agent composition was 100 mass %)]. The hair processing agent compositions of Examples 1 to 25 and Comparative Example 2 were obtained by mixing silica particles with an oil-soluble dispersant and then adding a surfactant and aqueous solvent thereto to form emulsions. Regarding the pH values of the hair processing agent compositions, the pH of Example 1 was 5.5, and the pH of Example 2 was 9.0. In the measurement of pH values, a pH meter produced by Horiba, Ltd., was used, and the measurement was performed by putting an electrode bar of the pH meter in each of the hair processing agent compositions prepared (putting the electrode bar into each emulsion).

The hair processing agent compositions of Examples 3 to 16 had a good silica-mixing state (expressed with “good” in Table 2). On the other hand, the hair processing agent composition of Comparative Example 2 had a bad silica-mixing state where silica floats to the emulsion surface (expressed with “Failed” in Table 2). Incidentally, the hair processing agent compositions of Examples 3 to 10 were used for the examples described below as Samples A to H.

(Hair Processing)

Each hair processing agent composition was applied to head hair or a hair bundle and left in a natural state for about 20 minutes, and then the head hair was washed with warm water and dried with a dryer. The evaluation result of the head hair after the hair processing is shown in Tables 1, 2, and 4.

TABLE 1

| | | | | | Surfactant | | | | | | | |
|---|---|---|---|---|---|---|---|---|---|---|---|---|
| | | | | | Sodium | | | | | | | |
| | | | Oil-soluble | | polyoxy- | | | | | | | |
| | | | dispersant | | ethylene | Aqueous solvent | | | Performance | | | |
| | Silica particle | | Cetanol | Lanoline | laurylether | Water | Triethanol | Total | Evaluation | | | |
| | Product | Content | (mass | alcohol | sulfate | (mass | amine | (mass | Bounce & | | | Sustain- |
| | name | (mass %) | %) | (mass %) | (mass %) | %) | (mass %) | %) | resilience | Rise | Stiffness | ability |
| Example 1 | AEROSIL- | 7.00 | 5.00 | 0.05 | 2.00 | 85.95 | 0.00 | 100.00 | Good | Excellent | Good | Excellent |
| | R812 | | | | | | | | | | | |
| Example 2 | AEROSIL- | 7.00 | 5.00 | 0.05 | 2.00 | 85.95 | 0.10 | 100.00 | Excellent | Excellent | Good | Excellent |
| | R812 | | | | | | | | | | | |

TABLE 2

| | | | | | | Propylene | Decamethyl | | | | | |
|---|---|---|---|---|---|---|---|---|---|---|---|---|
| | Silica Particles | | Ethanol | | | glycol | cyclopenta- | Total | Performance evaluation | | | |
| | Product | Content | (mass | Water | Glycerin | (PG) | Siloxane | (mass | Bounce & | | | Sustain- |
| | name | (mass %) | %) | (mass %) | (mass %) | (mass %) | (mass %) | %) | resilience | Rise | Stiffness | ability |
| Comparative. | AEROSIL- | 1.00 | 70.80 | 4.70 | 2.50 | 6.00 | 15.00 | 1.00 | Fair | Fair | Failed | Failed |
| Example. 1 | R812 | | | | | | | | | | | |

TABLE 3

| | | | | | | Surfactant | Aqueous solvent | |
|---|---|---|---|---|---|---|---|---|
| | | | | Oil-soluble | | Sodium | | Hydroxy- |
| | | | | dispersant | | polyoxy- | | ethane |
| | | Silica particle | | | Lanoline | laurylether | | diphosphonic |
| | | | Content | Cetanol | alcohol | sulfate | Water | acid |
| | | Product name | (mass %) | (mass %) | (mass %) | (mass %) | (mass %) | (mass %) |
| Example 3 | Sample A | AEROSIL-R812 | 0.50 | 6.23 | 0.06 | 2.49 | 89.30 | 0.249 |
| Example 4 | Sample B | AEROSIL-R812 | 1.00 | 6.20 | 0.06 | 2.48 | 88.84 | 0.248 |
| Example 5 | Sample C | AEROSIL-R812 | 2.00 | 6.14 | 0.06 | 2.45 | 87.98 | 0.245 |
| Example 6 | Sample D | AEROSIL-R812 | 3.00 | 6.07 | 0.06 | 2.43 | 87.04 | 0.243 |
| Example 7 | Sample E | AEROSIL-R812 | 5.00 | 5.95 | 0.06 | 2.38 | 85.25 | 0.238 |
| Example 8 | Sample F | AEROSIL-R812 | 7.00 | 5.82 | 0.06 | 2.33 | 83.46 | 0.233 |
| Example 9 | Sample G | AEROSIL-R812 | 10.00 | 5.63 | 0.06 | 2.25 | 80.73 | 0.225 |
| Example 10 | Sample H | AEROSIL-R812 | 12.00 | 5.51 | 0.06 | 2.20 | 78.95 | 0.220 |
| Example 11 | — | AEROSIL-R812 | 13.00 | 5.50 | 0.06 | 2.19 | 78.03 | 0.200 |
| Example 12 | — | AEROSIL-R812 | 14.00 | 5.49 | 0.06 | 2.18 | 77.01 | 0.200 |
| Example 13 | — | AEROSIL-R812 | 15.00 | 5.48 | 0.06 | 2.18 | 76.07 | 0.200 |
| Example 14 | — | AEROSIL-R812 | 16.00 | 5.47 | 0.06 | 2.18 | 75.01 | 0.200 |
| Example 15 | — | AEROSIL-R812 | 20.00 | 5.46 | 0.06 | 2.17 | 71.07 | 0.200 |
| Example 16 | — | AEROSIL-R812 | 22.00 | 5.44 | 0.06 | 2.16 | 69.06 | 0.199 |
| Comp. Ex. 2 | — | AEROSIL-R812 | 23.00 | 5.42 | 0.06 | 2.15 | 68.15 | 0.198 |

| | | Aqueous solvent | | | | |
|---|---|---|---|---|---|---|
| | | Disodium | | | | Evaluation |
| | | hydrogen- | Phosphoric | Salicylic | | Silica- |
| | | phosphate | acid | acid | Total | mixing |
| | | (mass %) | (mass %) | (mass %) | (mass %) | state |
| Example 3 | Sample A | 0.62 | 0.50 | 0.06 | 100.0 | Good |
| Example 4 | Sample B | 0.62 | 0.50 | 0.06 | 100.0 | Good |
| Example 5 | Sample C | 0.61 | 0.49 | 0.06 | 100.0 | Good |
| Example 6 | Sample D | 0.61 | 0.49 | 0.06 | 100.0 | Good |
| Example 7 | Sample E | 0.59 | 0.48 | 0.06 | 100.0 | Good |
| Example 8 | Sample F | 0.58 | 0.47 | 0.06 | 100.0 | Good |
| Example 9 | Sample G | 0.56 | 0.45 | 0.06 | 100.0 | Good |
| Example 10 | Sample H | 0.55 | 0.44 | 0.06 | 100.0 | Good |
| Example 11 | — | 0.55 | 0.44 | 0.06 | 100.0 | Good |
| Example 12 | — | 0.55 | 0.44 | 0.06 | 100.0 | Good |
| Example 13 | — | 0.55 | 0.44 | 0.06 | 100.0 | Good |
| Example 14 | — | 0.55 | 0.44 | 0.06 | 100.0 | Good |
| Example 15 | — | 0.55 | 0.44 | 0.06 | 100.0 | Good |
| Example 16 | — | 0.55 | 0.44 | 0.06 | 100.0 | Good |
| Comp. Ex. 2 | — | 0.55 | 0.44 | 0.06 | 100.0 | Failed |

TABLE 4

| | Hair processing agent composition | | | | | | | | | |
|---|---|---|---|---|---|---|---|---|---|---|
| | | | | | | | | | Surfactant | |
| | | | | | | | | | | Sodium |
| | | | | | | | | | | polyoxy- |
| | | | Oil-soluble dispersant | | | | | | Sodium | ethylene |
| | | | | Lanoline | Coto- | | | Hardening | lauryl- | lauryl- |
| | Silica particle | | | alcohol | stearyl | Stearyl | | rapeseed oil | glycol | ether |
| | Product | Content | Cetanol | (mass | alcohol | alcohol | Stearic acid | alcohol | acetate | sulfate |
| | name | (mass %) | (mass %) | %) | (mass %) | (mass %) | (mass %) | (mass %) | (mass %) | (mass %) |
| Ex. | AEROSILR812 | 0.69 | 0.00 | 9.93 | 0.00 | 0.00 | 0.00 | 0.00 | 19.86 | 0.00 |
| 17 | | | | | | | | | | |
| Ex. | AEROSILR812 | 0.69 | 0.00 | 0.00 | 9.93 | 0.00 | 0.00 | 0.00 | 19.86 | 0.00 |
| 18 | | | | | | | | | | |
| Ex. | AEROSILR812 | 0.69 | 0.00 | 0.00 | 0.00 | 9.93 | 0.00 | 0.00 | 19.86 | 0.00 |
| 19 | | | | | | | | | | |
| Ex. | AEROSILR812 | 0.69 | 0.00 | 0.00 | 0.00 | 0.00 | 9.93 | 0.00 | 19.86 | 0.00 |
| 20 | | | | | | | | | | |
| Ex. | AEROSILR812 | 0.69 | 0.00 | 0.00 | 0.00 | 0.00 | 0.00 | 9.93 | 19.86 | 0.00 |
| 21 | | | | | | | | | | |
| Ex. | AEROSILR812 | 5.00 | 0.00 | 0.00 | 50.00 | 0.00 | 0.00 | 0.00 | 2.00 | 0.00 |
| 22 | | | | | | | | | | |
| Ex. | AEROSILR812 | 5.00 | 0.00 | 0.00 | 0.00 | 50.00 | 0.00 | 0.00 | 2.00 | 0.00 |
| 23 | | | | | | | | | | |
| Ex. | AEROSILR812 | 5.00 | 0.00 | 0.00 | 0.00 | 0.00 | 50.00 | 0.00 | 2.00 | 0.00 |
| 24 | | | | | | | | | | |
| Ex. | AEROSILR812 | 5.00 | 0.50 | 0.05 | 0.00 | 0.00 | 0.00 | 0.00 | 0.00 | 2.00 |
| 25 | | | | | | | | | | |

| | Hair processing | | | | | | |
|---|---|---|---|---|---|---|---|
| | agent composition | | | | | | |
| | Aqueous | | | | | | |
| | solvent | | Performance Evaluation | | | | |
| | Water | Total | Bounce & | | | | |
| | (mass %) | (mass %) | resilience | Rise | Stiffness | Sustainability | Voluminousness |
| Ex. | 69.50 | 100.0 | Excel. | Excel. | Good | Excel. | Excel. |
| 17 | | | | | | | |
| Ex. | 69.50 | 100.0 | Excel. | Excel. | Good | Excel. | PP |
| 18 | | | | | | | |
| Ex. | 69.50 | 100.0 | Excel. | Excel. | Good | Excel. | Excel. |
| 19 | | | | | | | |
| Ex. | 69.50 | 100.0 | Excel. | Excel. | Good | Excel. | Excel. |
| 20 | | | | | | | |
| Ex. | 69.50 | 100.0 | Good | Fair | Excel. | Fair | Fair |
| 21 | | | | | | | |
| Ex. | 43.00 | 100.0 | Excel. | Excel. | Good | Excel. | Excel. |
| 22 | | | | | | | |
| Ex. | 43.00 | 100.0 | Excel. | Excel. | Good | Excel. | Excel. |
| 23 | | | | | | | |
| Ex. | 43.00 | 100.0 | Excel. | Good | Good | Good | Excel. |
| 24 | | | | | | | |
| Ex. | 92.45 | 100.0 | Fair | Fair | Fair | Fair | Fair |
| 25 | | | | | | | |

Electron micrographs were taken regarding unprocessed head hair and head hair after hair processing using each of the hair processing agent compositions of Examples 1 and 2 and Comparative Example 1.

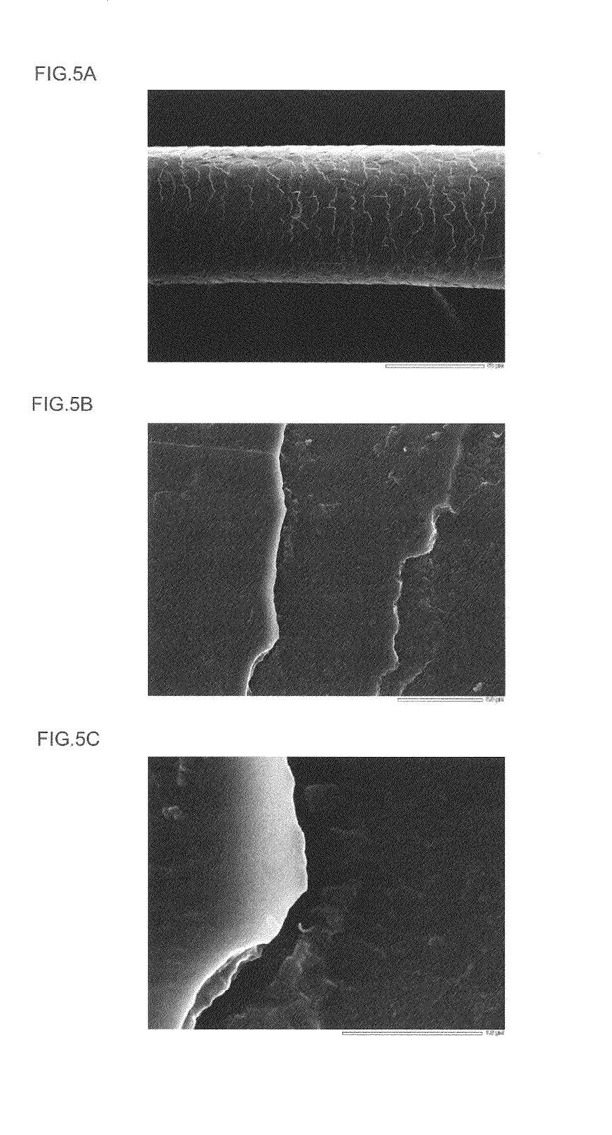

Regarding the unprocessed head hair, there are shown the whole image (FIG. 5A), an enlarged image of cuticles (FIG. 5B), and an enlarged image of a cuticle edge portion (FIG. 5C). In these photographs, the direction from left to right is the direction from the root side to the tip side of head hair. As shown in FIG. 5A, a scale structure covered with a large number of cuticles is formed on the surface of head hair. As shown in FIGS. 5B and 5C, the scale structure is formed in such a manner that each cuticle covers the root-side portion of an another adjacent cuticle on the tip side of the each cuticle.

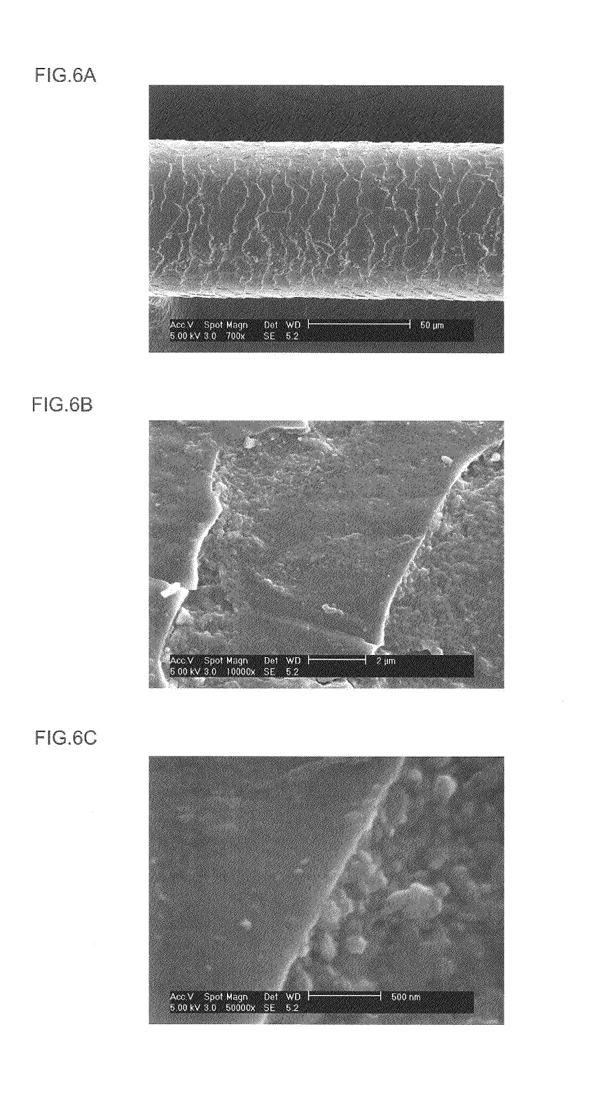

Regarding a head hair after hair processing using the hair processing agent composition of Example 1, there are shown the whole image (FIG. 6A), an enlarged image of cuticles (FIG. 6B), and an enlarged image of a cuticle edge portion (FIG. 6C). In the head hair of Example 1, there was observed a state where silica particles stayed inside the gap between a cuticle edge portion exposed to the outside and a cuticle covered with the cuticle edge portion and adjacently located on the tip side of the outside cuticle (FIGS. 6B and 6C). In addition, in the head hair of Example 1, a few silica particles scattered on the surface in the vicinity of the cuticle edge.

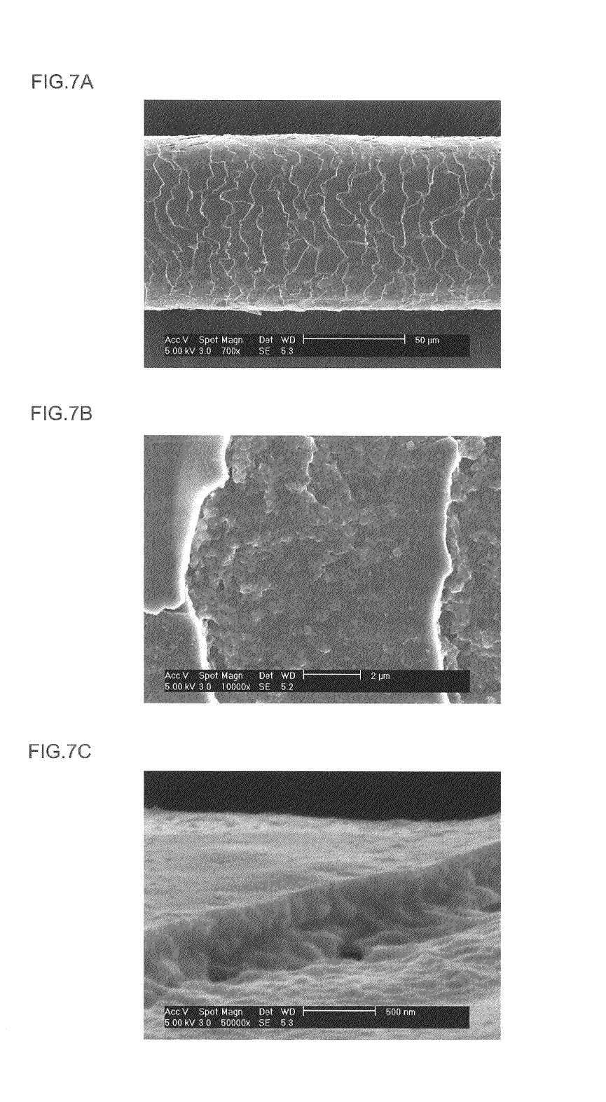

Regarding a head hair after hair processing using the hair processing agent composition of Example 2, there are shown the whole image (FIG. 7A), an enlarged image of cuticles (FIG. 7B), and an enlarged image of a cuticle edge portion (FIG. 7C). In the head hair of Example 2, there was observed a state where silica particles stayed inside the gap between a cuticle edge portion exposed to the outside and an adjacent cuticle covered with this and located on the tip side (FIGS. 7B and 7C). In comparison with Example 1, Example 2 had a tendency to allow a larger amount of silica particles to stay in the gap between cuticles. In addition, in the head hair of Example 2, a few silica particles scattered on the surface in the vicinity of the cuticle edge.

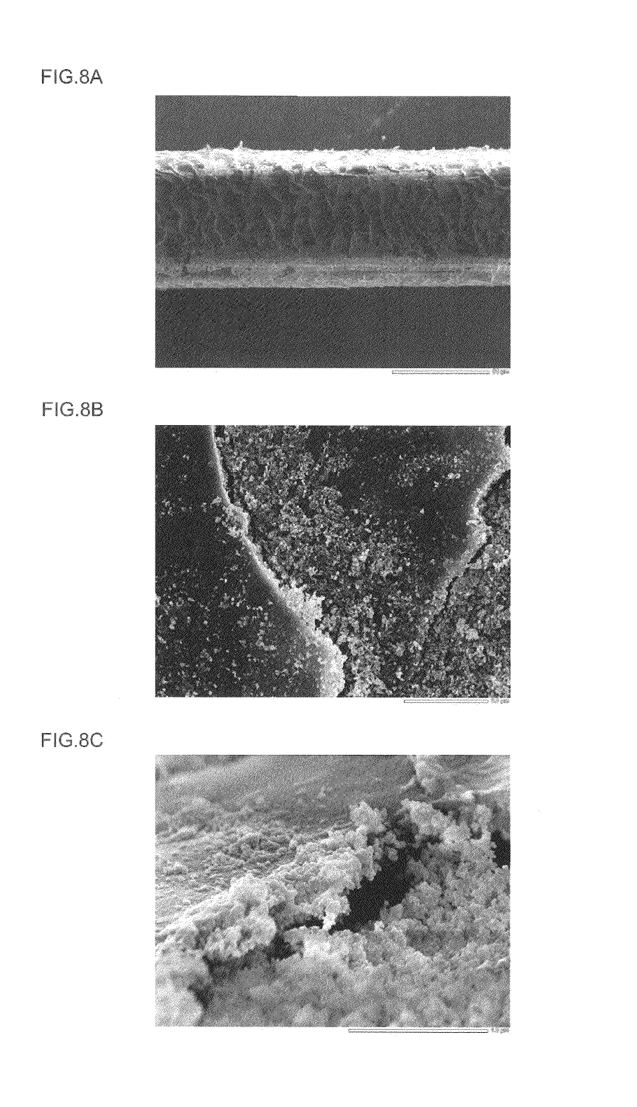

Regarding a head hair after hair processing using the hair processing agent composition of Comparative Example 1, there are shown the whole image (FIG. 8A), an enlarged image of cuticles (FIG. 8B), and an enlarged image of a cuticle edge portion (FIG. 8C). In the head hair of Comparative Example 1, there was observed a state where silica particles got upon the outside surface of a cuticle edge portion (FIG. 8C). Furthermore, in the head hair of Comparative Example 1, the cuticle edge portion exposed to the outside and a cuticle covered with the cuticle edge portion and adjacently located on the tip side adhered to each other closely, and there could not be observed a state where silica particles stayed inside the gap between the cuticles (FIG. 8B). In addition, in the head hair of the Comparative Example 1, there was observed a state where a large amount of silica particles covered the surface of the cuticle (FIGS. 8A to 8C).

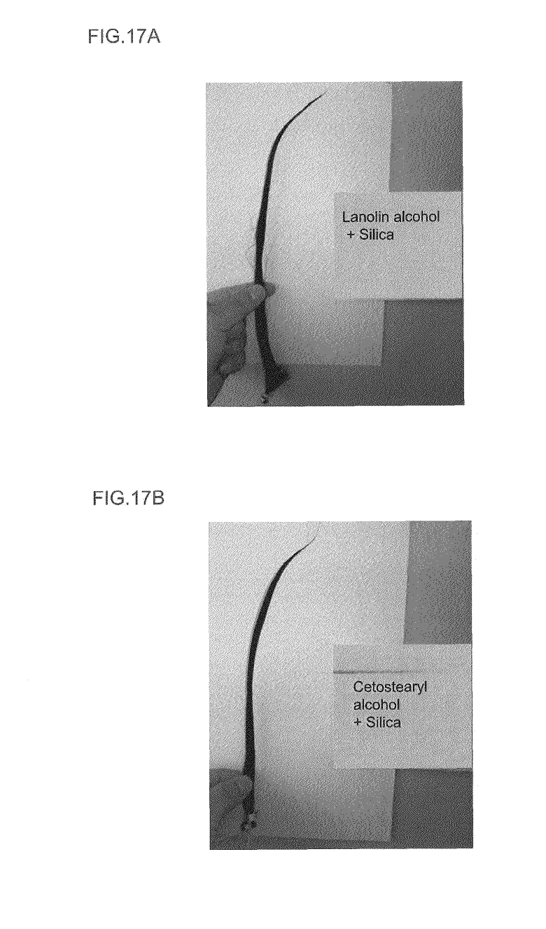

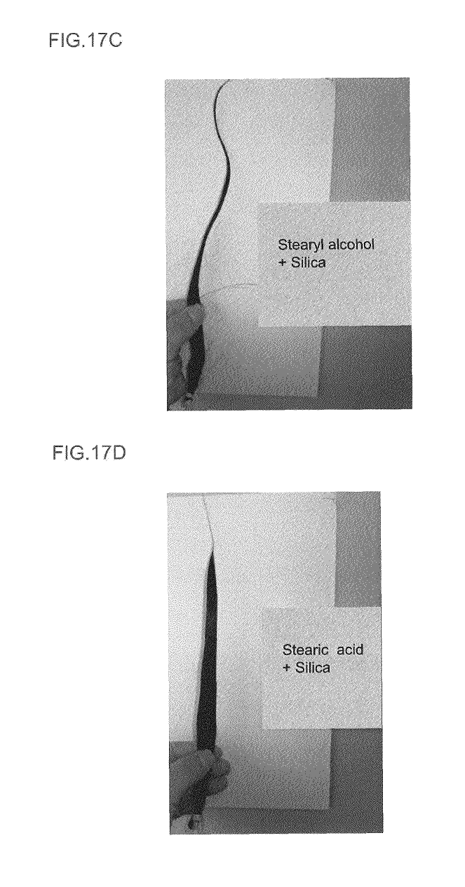

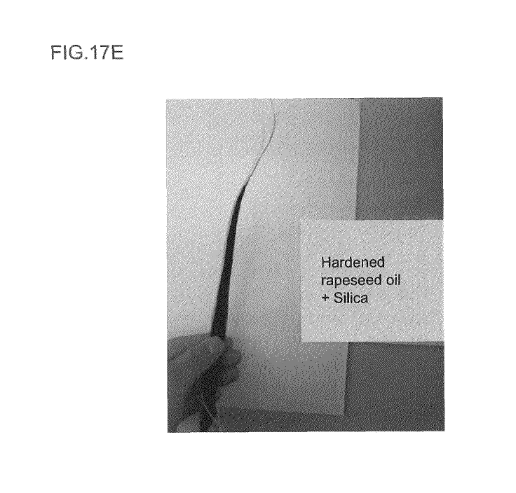

Photos of a hair bundle subjected to processing with the hair processing agent compositions of Examples 17 to 21 are shown in FIGS. 17A to 17E.

### (4) Hair Processing Agent Composition for Dyeing Hair

### (4-1) Oxidation Hair Dye

### Examples 26 to 29 and 31 to 35

The hair processing agent compositions for dyeing hair of Examples 26 to 29 and Examples 31 to 35 were prepared by blending the aforementioned materials to have compositions shown in Tables 5, 7, and 8. Note that, the second agent of each of Examples 26 to 29 and Examples 31 to 35 was obtained by firstly mixing silica particles with an oil-soluble dispersant and, then, adding a surfactant, an aqueous solvent, and hydrogen peroxide thereto to form an emulsion. Just before use, an equal amount (100 g of each) of the first agent and the second agent were mixed and stirred well into a creamy consistency.

### Example 30

The hair processing agent composition for dyeing hair of Example 30 was prepared by blending the aforementioned materials to have compositions shown in Table 6. Note that, the first agent of Example 30 was obtained by firstly mixing silica particles with an oil-soluble dispersant, and then adding a surfactant and an aqueous solvent thereto to form an emulsion, to which Wella Koleston Perfect J was further added. Just before use, 45 g of the first agent and 40 g of the second agent were mixed and stirred well into a creamy consistency.

### Comparative Example 3

The hair processing agent composition for dyeing hair of the Comparative Example 3 was prepared by blending the aforementioned materials to have compositions shown in Table 9. Just before use, an equal amount (100 g of each) of the first agent and the second agent were mixed and stirred well into a creamy consistency.

(Dyeing Hair)

Using a brush or a comb, the hair processing agent composition for dyeing hair was applied to head hair evenly from the root to the tip of the hair. After the application, it was left in a natural state for 20 to 30 minutes to allow the oxidation by the hair dye. After confirming that the hair was dyed, while immersing the head hair in lukewarm water, unwanted extraneous matter adhering to the surface of the head hair was taken off. Further, the head hair was rinsed with warm water, shampooed by the use of a shampoo (shampoo agent). Next, treatment of the head hair was performed by the use of a hair treatment agent. Subsequently, water on the head hair was removed with a towel, and the head hair was dried with a drier.

The evaluation results of the head hair after dyeing are shown in Table 5 (Examples 26 to 29), Table 6 (Example 30), Table 7 (Examples 31 to 34), Table 8 (Example 35), and Table 9 (Comparative Example 3).

In any of the hair processing agent compositions for dyeing hair of Examples 26 to 29, almost no stimulus to the scalp, which is often caused upon using the conventional hair dyeing agent (upon general use of “Wella Koleston Perfect J, 6 brightness· black (6/0)” (P&G)), was felt (expressed as “none” in the column of “stimulus to scalp” in Table 5). However, a stimulus to the scalp was sometimes felt by the hair processing agent composition for dyeing hair of Example 30 (expressed as “present” in the column of “stimulus to scalp” in Table 6).

Regarding the “dyeing result”, “excellent” was given to the case of having a beautiful dyeing result as in the general use of the “Wella Koleston Perfect J, 6 brightness•black (6/0)” (P&G). “Good” was given to the case of having a slightly lighter dyeing result in comparison with the general use of the “Wella Koleston Perfect J, 6 brightness•black (6/0)” (P&G). “Fair” was given to the case of having an uneven dyeing result though the hair was dyed.

TABLE 5

| | | Hair processing agent composition for dyeing hair | | | | | | | | | |
|---|---|---|---|---|---|---|---|---|---|---|---|
| | | Second agent | | | | | | | | | |
| | First | | | | | Surfactant | | | | | |
| | agent | | | | | Sodium | Aqueous solvent | | | | |
| | Wella | | | Oil-soluble | | polyoxy- | | Hydroxy- | | | |
| | Koleston | | | dispersant | | ethylene | | ethane | Disodium | Phos- | |
| | Perfect J6/0 | Silica particle | | | Lanoline | lauryl- | | diphosphonic | hydrogen- | phoric | Salicylic |
| | (P&G) | Product | | Cetanol | alcohol | ether | Water | acid | phosphate | acid | acid |
| | (g) | name | (g) | (g) | (g) | sulfate (g) | (g) | (g) | (g) | (g) | (g) |
| Ex. | 100.00 | AEROSILR812 | 2.00 | 5.00 | 0.05 | 2.00 | 72.70 | 0.20 | 0.50 | 0.40 | 0.05 |
| 26 | | | | | | | | | | | |
| Ex. | 100.00 | AEROSILR812 | 5.00 | 5.00 | 0.05 | 2.00 | 69.70 | 0.20 | 0.50 | 0.40 | 0.05 |
| 27 | | | | | | | | | | | |
| Ex. | 100.00 | AEROSILR812 | 7.00 | 5.00 | 0.05 | 2.00 | 67.70 | 0.20 | 0.50 | 0.40 | 0.05 |
| 28 | | | | | | | | | | | |
| Ex. | 100.00 | AEROSILR812 | 10.00 | 5.00 | 0.05 | 2.00 | 64.70 | 0.20 | 0.50 | 0.40 | 0.05 |
| 29 | | | | | | | | | | | |

| | Hair processing | | | | | | | |
|---|---|---|---|---|---|---|---|---|
| | agent composition | | | | | | | |
| | for dyeing hair | | | | | | | |
| | Second agent | | Performance evaluation | | | | | |
| | Hydrogen | Total | Bounce | | | | | Stimulus |
| | peroxide | mass | and | | | | Dyeing | to |
| | (g) | (g) | Resilience | Rise | Stiffness | Sustainability | result | scalp |
| Ex. | 17.10 | 100.00 | Excel. | Excel. | Excel. | Excel. | Excel. | None |
| 26 | | | | | | | | |
| Ex. | 17.10 | 100.00 | Excel. | Excel. | Excel. | Excel. | Excel. | None |
| 27 | | | | | | | | |
| Ex. | 17.10 | 100.00 | Excel. | Excel. | Good | Excel. | Good | None |
| 28 | | | | | | | | |
| Ex. | 17.10 | 100.00 | Good | Good | Fair | Excel. | Good | None |
| 29 | | | | | | | | |

TABLE 6

| | Hair processing agent composition for dyeing hair | | | | | | | | | |
|---|---|---|---|---|---|---|---|---|---|---|
| | First agent | | | | | | | | | |
| | | | | | | | | Oxidation | | |
| | | | | | Surfactant | | | dye | | |
| | | | Oil-soluble | | Sodium | | | Wella | | |
| | | | dispersant | | polyoxy- | Aqueous solvent | | Koleston | | Second agent |
| | Silica particle | | | Lanoline | ethylene lauryl- | | Triethanol | Perfect J | | Welloxon |
| | Product | Content | Cetanol | alcohol | ether sulfate | Water | amine | (P&G) | Total mass | Perfect (P&G) |
| | name | (g) | (g) | (g) | (g) | (g) | (g) | (g) | (g) | (g) |
| Ex. 30 | AEROSILR812 | 0.35 | 0.25 | 0.0025 | 0.10 | 4.2925 | 0.005 | 40.00 | 45.00 | 40.00 |

| | Performance evaluation | | | | | |
|---|---|---|---|---|---|---|
| | Bounce and | | | | Dyeing | Stimulus to |
| | Resilience | Rise | Stiffness | Sustainability | result | scalp |
| Ex. 30 | Good | Excellent | Excellent | Excellent | Excellent | Present |

TABLE 7

| | Hair processing agent composition for dyeing hair | | | | | | | | | | |
|---|---|---|---|---|---|---|---|---|---|---|---|
| | | Second agent | | | | | | | | | |
| | First agent | | | | | Surfactant | Aqueous solvent | | | | |
| | Wella | | | | | Sodium | | Hydroxy- | | | |
| | Koleston | | | Oil-soluble dispersant | | polyoxy- | | ethane | Disodium | | |
| | Perfect | Silica particle | | | Lanoline | laurylether | | diphosphonic | hydrogen- | Phosphonic | Salicylic |
| | (P&G) | Product | Content | Cetanol | alcohol | sulfate | Water | acid | phosphate | acid | acid |
| | (g) | name | (mass %) | (mass %) | (mass %) | (mass %) | (mass %) | (mass %) | (mass %) | (mass %) | (mass %) |
| Ex. 31 | 100.0 | AEROSILR812 | 5.00 | 4.90 | 0.049 | 1.96 | 70.19 | 0.49 | 0.39 | 0.049 | 16.74 |
| Ex. 32 | 100.0 | AEROSILR812 | 7.00 | 4.75 | 0.475 | 1.90 | 68.52 | 0.49 | 0.38 | 0.048 | 16.25 |
| Ex. 33 | 100.0 | AEROSILR812 | 10.00 | 4.60 | 0.046 | 1.84 | 66.72 | 0.46 | 0.37 | 0.046 | 15.73 |
| Ex. 34 | 100.0 | AEROSILR812 | 12.00 | 4.50 | 0.045 | 1.80 | 65.20 | 0.45 | 0.39 | 0.045 | 15.39 |
| Comp. | 100.0 | — | — | 0 | 0 | 0 | 0 | 0 | 0 | 0 | 0 |
| Ex. 3 | | | | | | | | | | | |

| | Hair processing | | | | | | | |
|---|---|---|---|---|---|---|---|---|
| | agent composition | | | | | | | |
| | for dyeing hair | | | | | | | |
| | Second agent | | | | | | | |
| | Hydrogen | | Performance evaluation | | | | | |
| | peroxide | Total mass | Bounce | | | | Dyeing | |
| | (mass %) | (g) | and resilience | Rise | Stiffness | Sustainability | result | Voluminousness |
| Ex. 31 | 0.20 | 100.0 | Excel. | Excel. | Good | Good | Excel. | Excel. |
| Ex. 32 | 0.19 | 100.0 | Excel. | Excel. | Fair | Good | Good | Excel. |
| Ex. 33 | 0.18 | 100.0 | Excel. | Excel. | Fair | Good | Good | Excel. |
| Ex. 34 | 0.18 | 100.0 | Excel. | Excel. | Fair | Good | Fair | Excel. |
| Comp. | 0 | 0 | Failed | Failed | Excel. | Good | Good | Fair |
| Ex. 3 | | | | | | | | |

TABLE 8

| | Hair processing agent composition for dyeing hair | | | | | | | | | |
|---|---|---|---|---|---|---|---|---|---|---|
| | | Second agent | | | | | | | | |
| | First agent | | | | | | Aqueous solvent | | | |
| | In-fit | | | Oil-soluble | | | | Hydroxy- | | |
| | Illumiant | | | dispersant | | Surfactant | | ethane | Disodium | |
| | chocolate | Silica particle | | | Lanoline | Sodium polyoxy- | | diphosphonic | hydrogen- | Phosphoric |
| | blown 5 | Product | Content | Cetanol | alcohol | laurylether sulfate | Water | acid | phosphate | acid |
| | (g) | name | (mass %) | (mass %) | (mass %) | (mass %) | (mass %) | (mass %) | (mass %) | (mass %) |
| Ex. 35 | 100.0 | AEROSILR812 | 3.00 | 4.5 | 0.045 | 1.80 | 74.20 | 0.45 | 0.39 | 0.045 |

| | Hair processing agent composition | | | | | | | | |
|---|---|---|---|---|---|---|---|---|---|
| | for dyeing hair | | | | | | | | |
| | Second agent | | | | | | | | |
| | Aqueous solvent | Hydrogen | | Performance evaluation | | | | | |
| | Salicylic acid | peroxide | Total mass | Bounce and | | | | Dyeing | |
| | (mass %) | (mass %) | (g) | resilience | Rise | Stiffness | Sustainability | result | Voluminousness |
| Ex. 35 | 15.39 | 0.18 | 100.0 | Excel. | Excel. | Good | Good | Excel. | Excel. |

TABLE 9

| | Hair processing | | | | | | | |
|---|---|---|---|---|---|---|---|---|
| | agent | | | | | | | |
| | composition for | | | | | | | |
| | dyeing hair | | | | | | | |
| | First agent | | | | | | | |
| | Wella | Second | | | | | | |
| | Koleston | agent | | | | | | |
| | Perfect | Welloxon | Performance evaluation | | | | | |
| | 6/0 | Perfect | Bounce and | | | | | |
| | (g) | (g) | resilience | Rise | StiffNess | Sustainability | Dyeing result | Voluminousness |
| Comp. | 100.0 | 100.0 | Failed | Failed | Excellent | Good | Good | Fair |
| Ex. 3 | | | | | | | | |

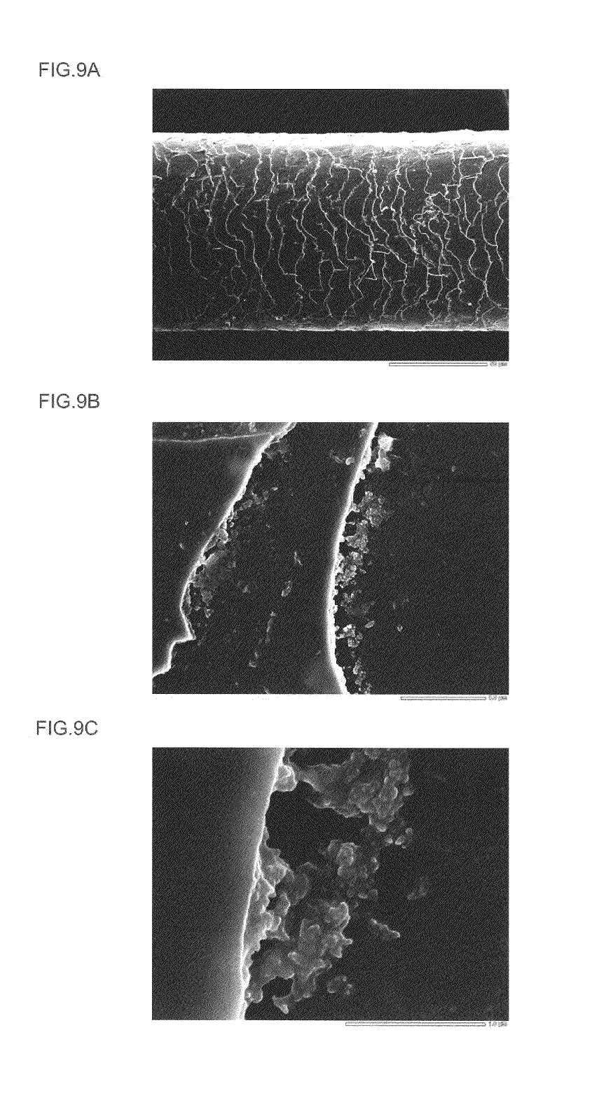

Regarding a head hair after hair processing using the hair processing agent composition for dyeing hair of Example 28, there are shown the whole image (FIG. 9A), an enlarged image of cuticles (FIG. 9B), and an enlarged image of a cuticle edge portion (FIG. 9C). In the head hair of Example 28, there was observed a state where silica particles stayed inside the gap between a cuticle edge portion exposed to the outside and a cuticle covered with the cuticle edge portion and adjacently located on the tip side of the outside cuticle (FIGS. 9B and 9C). In addition, in the head hair of Example 28, almost no silica particle was observed on the surface of the cuticle.

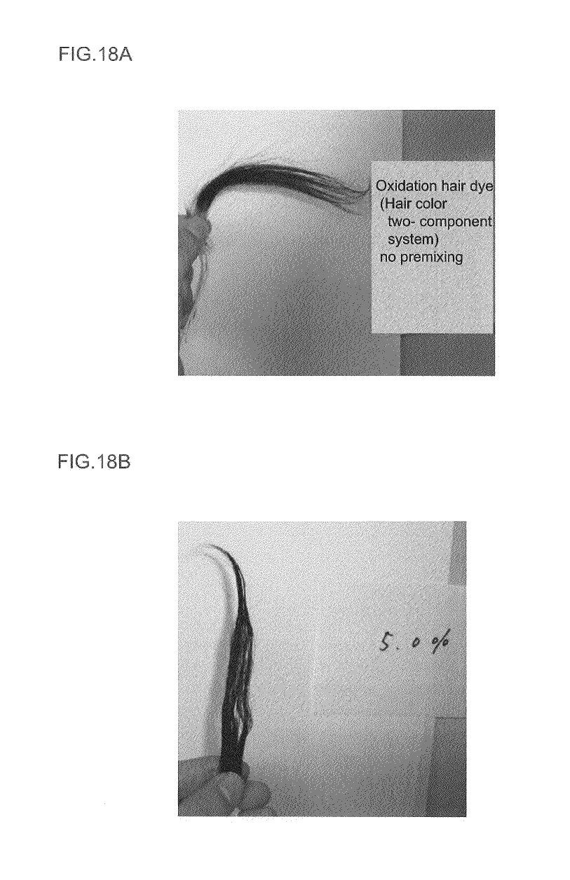

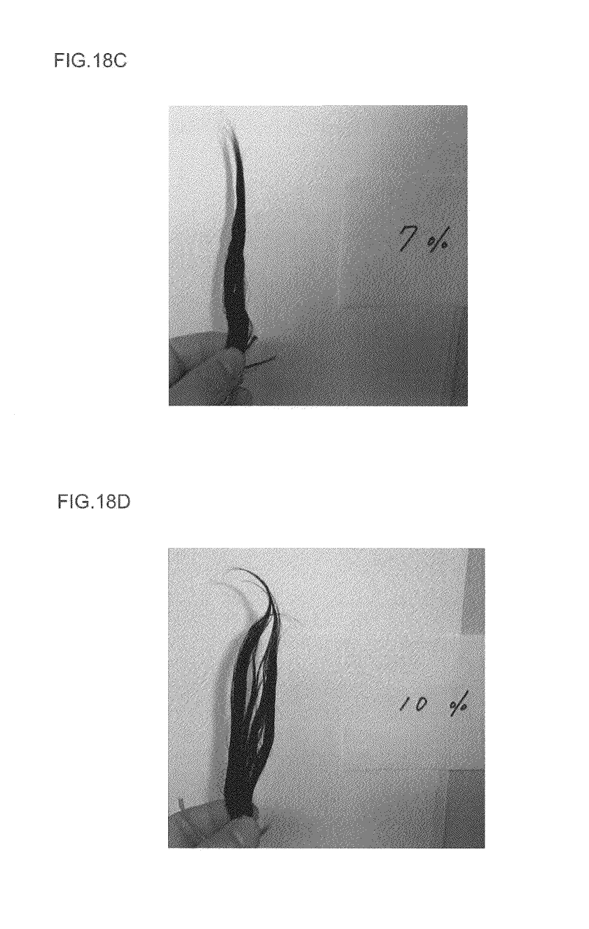

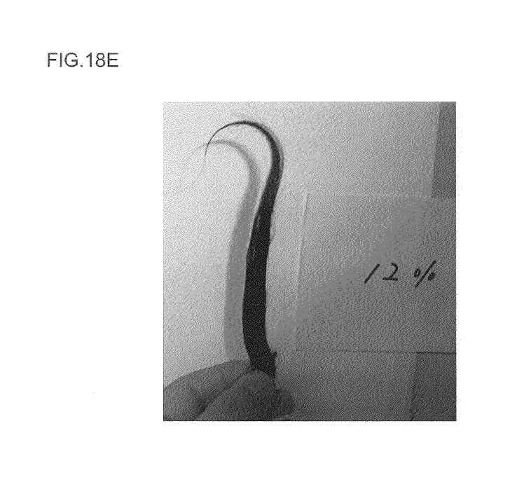

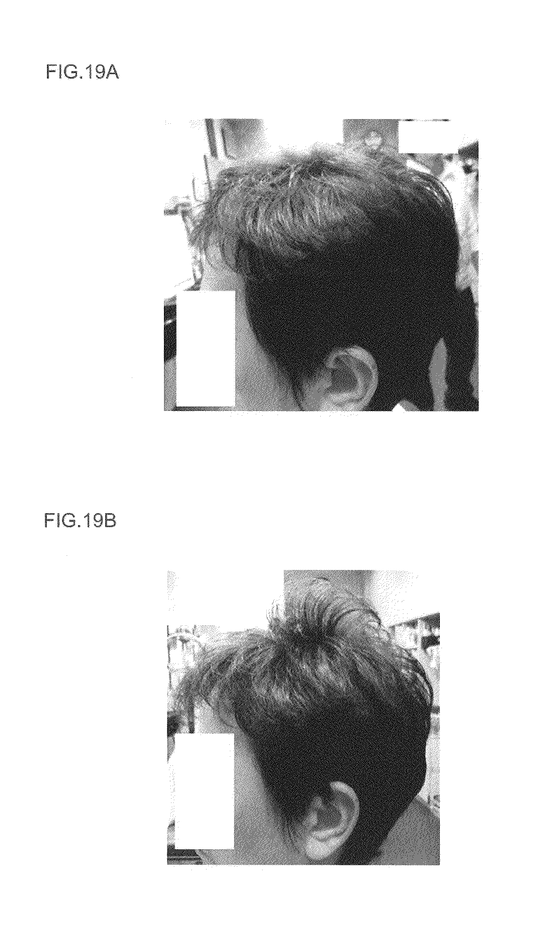

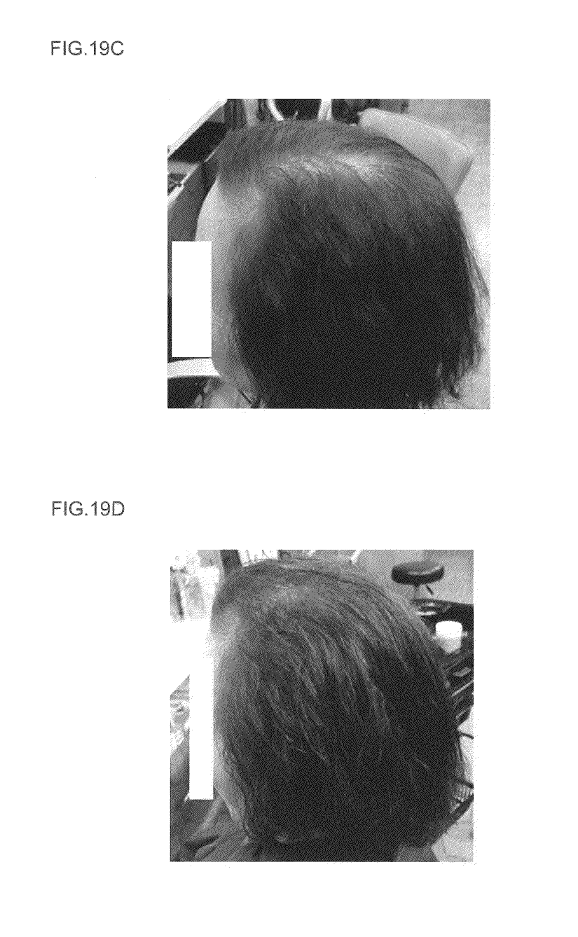

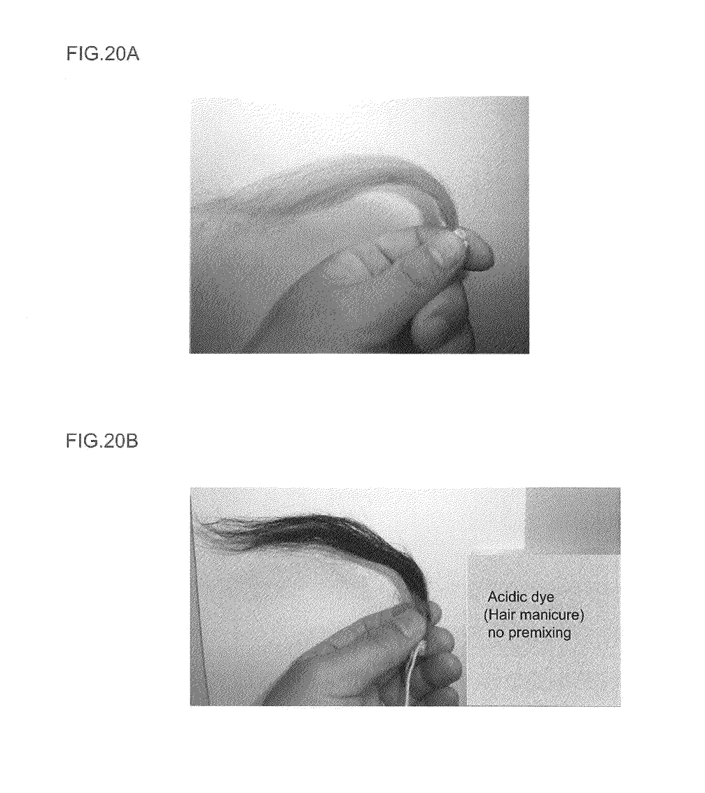

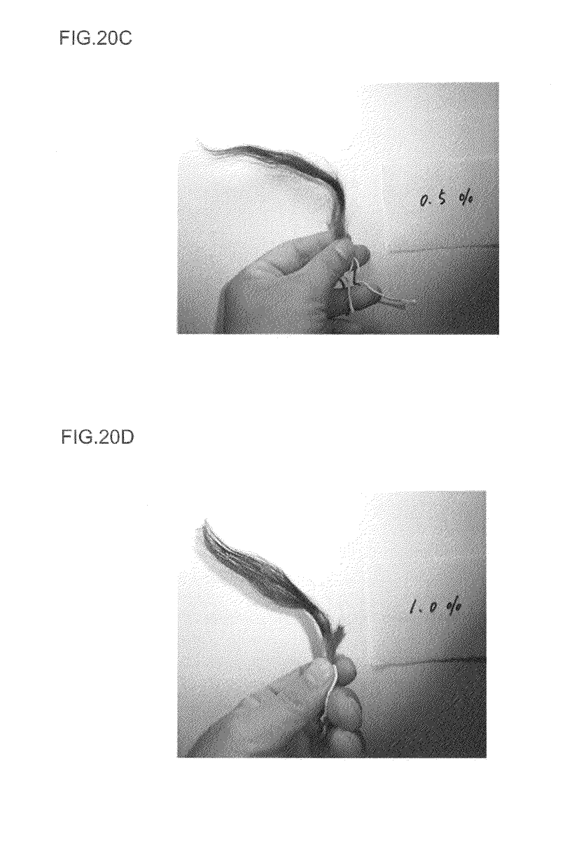

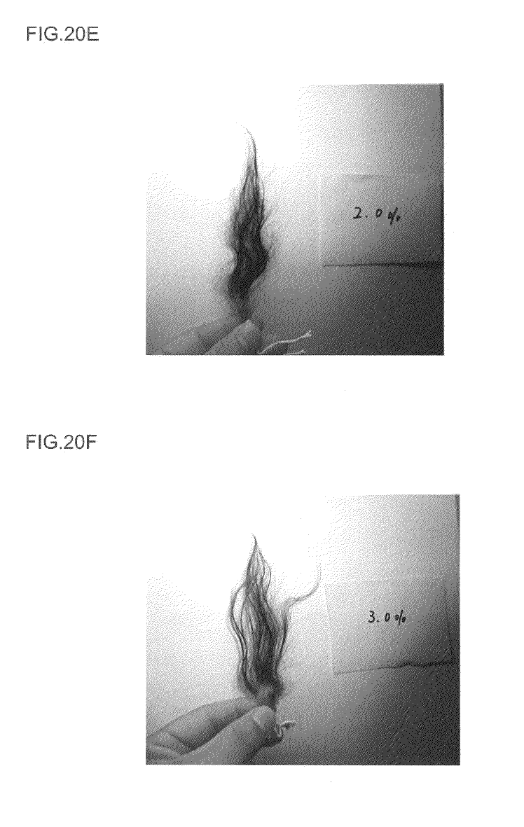

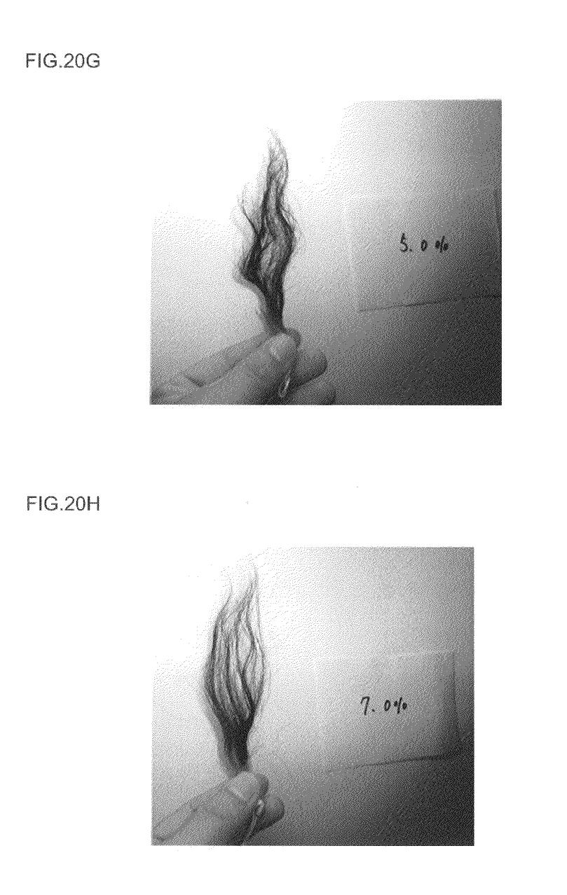

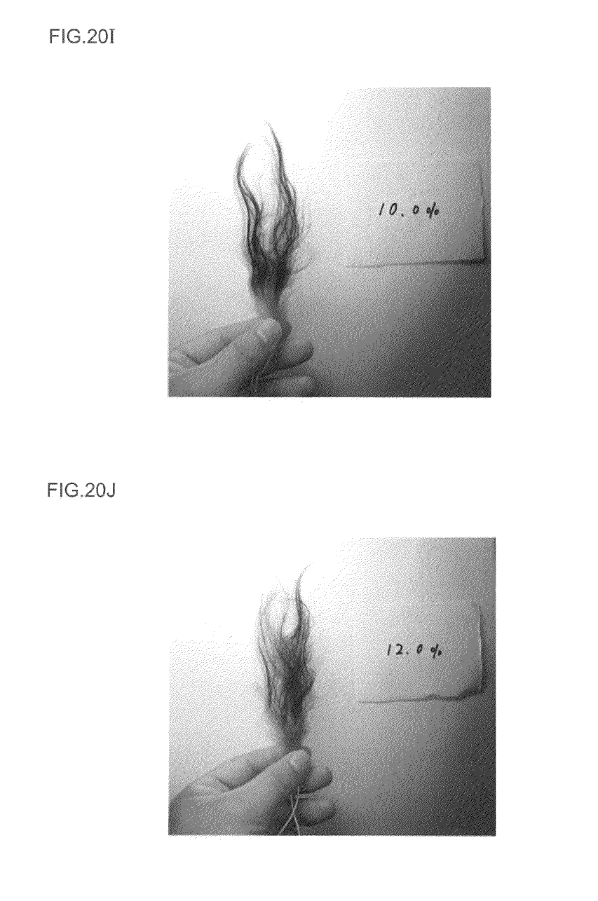

Photos of hair bundles subjected to processing with the hair processing agent compositions for dyeing hair of Comparative Example 3 and Examples 31 to 34 are shown in FIGS. 18A to 18E. Note that unprocessed hair bundles were the same as shown in the photo of FIG. 20A. FIG. 19A is a photo of the test subject A before the processing. FIG. 19B is a photo of the test subject A after subjecting to the processing with the hair processing agent composition for dyeing hair of Example 35. FIG. 19C is a photo of the head of the test subject B before the processing. FIG. 19D is a photo of the head of the test subject B after subjecting to the processing with the hair processing agent composition for dyeing hair of Example 35.

### (4-2) Acidic Dye

### Examples 36 to 43, Comparative Example 4

The hair processing agent compositions for dyeing hair of Examples 36 to 43 and Comparative Example 4 were prepared by blending the aforementioned materials to have compositions shown in Table 10.

Regarding the “dyeing result”, “excellent” was given to the case of having a beautiful dyeing result as in the general use of “Wella Color FreshBlack 0” (P&G). “Good” was given to the case of having a slightly lighter dyeing result in comparison with the general use of the “Wella Color Fresh Black 0” (P&G). “Fair” was given to the case of having an uneven dyeing result though the hair was dyed.

A photo of an unprocessed bundle of hair is shown in FIG. 20A, and photos of bundles of hair subjected to the processing with the hair processing agent compositions for dyeing hair of Comparative Example 4 and Examples 36 to 43 are shown in FIGS. 20B to 20J.

TABLE 10

| | Hair processing agent | | | | | | | | |
|---|---|---|---|---|---|---|---|---|---|
| | composition for dyeing hair | | | | | | | | |
| | Hair manicure | | | | | | | | |
| | (acidic dye) | Hair processing | | | | | | | |
| | Wella Color Fresh | agent composition | | Performance evaluation | | | | | |
| | black 0 | | Content | Bounce and | | | | | |
| | (g) | Kind | (g) | resilience | Rise | Stiffness | Sustainability | Dyeing result | Voluminousness |
| Example 36 | 100.0 | Sample A | 100.0 | Good | Good | Excellent | Good | Excellent | Good |
| Example 37 | 100.0 | Sample B | 100.0 | Good | Good | Excellent | Good | Excellent | Good |
| Example 38 | 100.0 | Sample C | 100.0 | Good | Good | Excellent | Good | Excellent | Good |
| Example 39 | 100.0 | Sample D | 100.0 | Good | Good | Excellent | Good | Excellent | Good |
| Example 40 | 100.0 | Sample E | 100.0 | Good | Good | Excellent | Good | Excellent | Good |
| Example 41 | 100.0 | Sample F | 100.0 | Good | Good | Good | Good | Good | Good |
| Example 42 | 100.0 | Sample G | 100.0 | Excellent | Excellent | Good | Good | Fair | Excellent |
| Example 43 | 100.0 | Sample H | 100.0 | Excellent | Excellent | Good | Good | Fair | Excellent |
| Comp. Ex. 4 | 100.0 | — | — | Failed | Failed | Excellent | Good | Excellent | Failed |

### (4-3) HC•Basic Dye

### Examples 44 to 51, Comparative Example 5

The hair processing agent compositions for dyeing hair of Examples 44 to 51 and Comparative Example 5 were prepared by blending the aforementioned materials to have the compositions shown in Table 11.

Regarding the “dyeing result”, “excellent” was given to the case of having a beautiful dyeing result as in the general use of the “BC Hair Color Treatment BL” (color treatment for airy finishing) (Medicos Co. Ltd.). “Good” was given to the case of having a slightly light dyeing result in comparison with the general use of the “BC Hair Color Treatment BL” (color treatment for airy finishing) (Medicos Co. Ltd.). “Fair” was given to the case of having an uneven dyeing result though the hair was dyed.

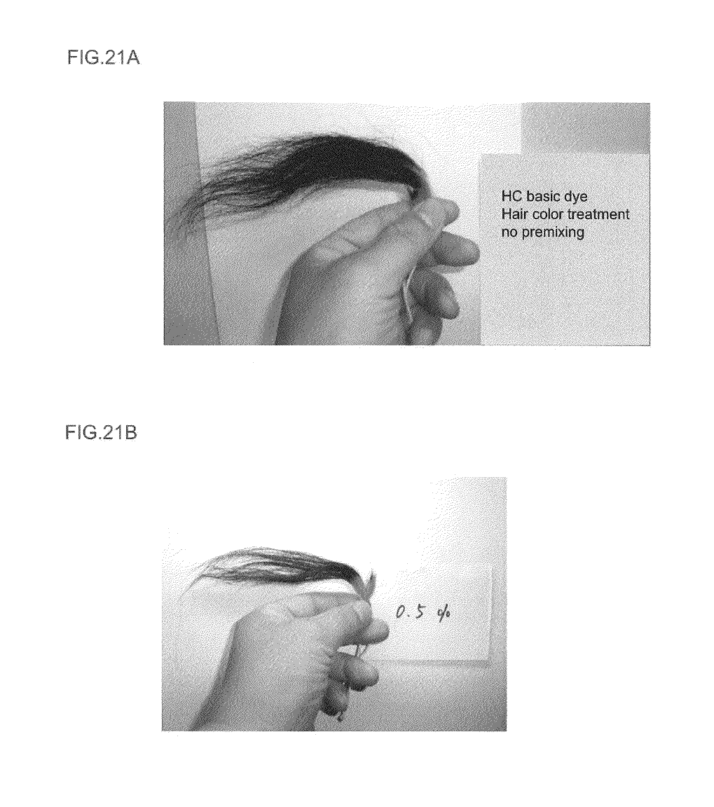

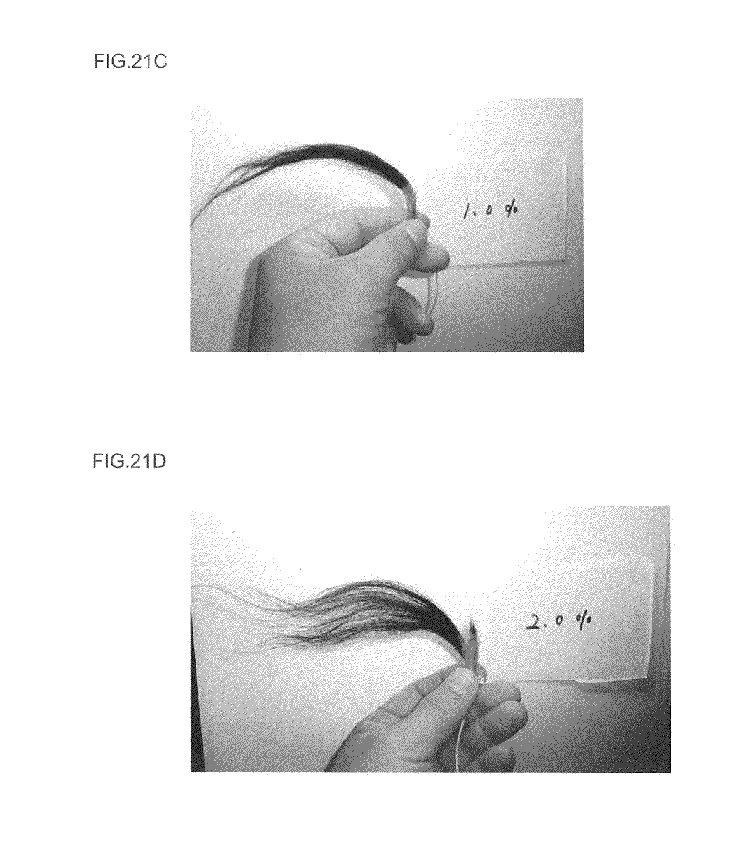

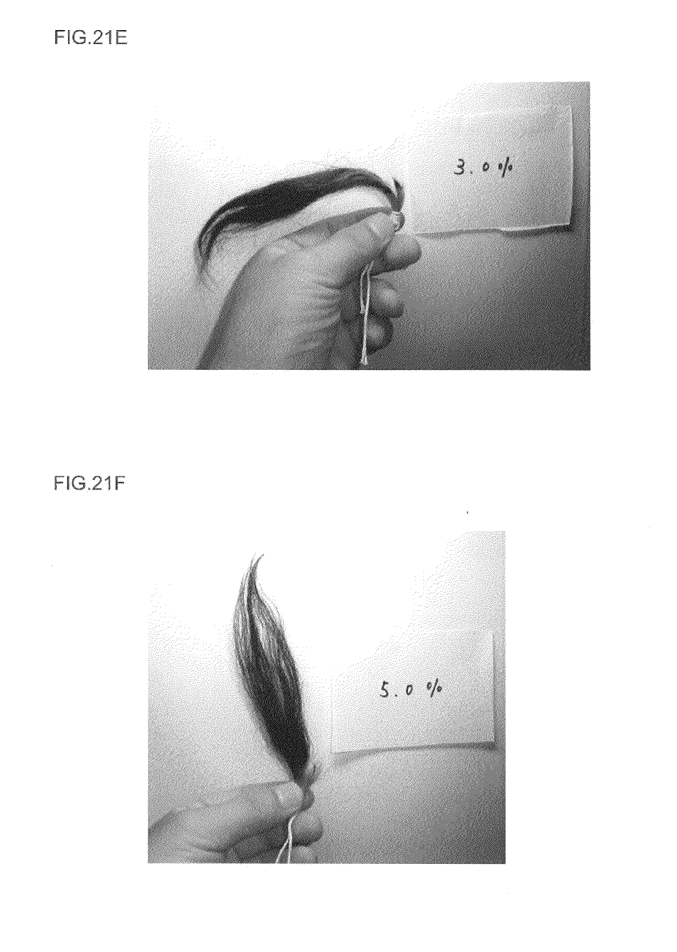

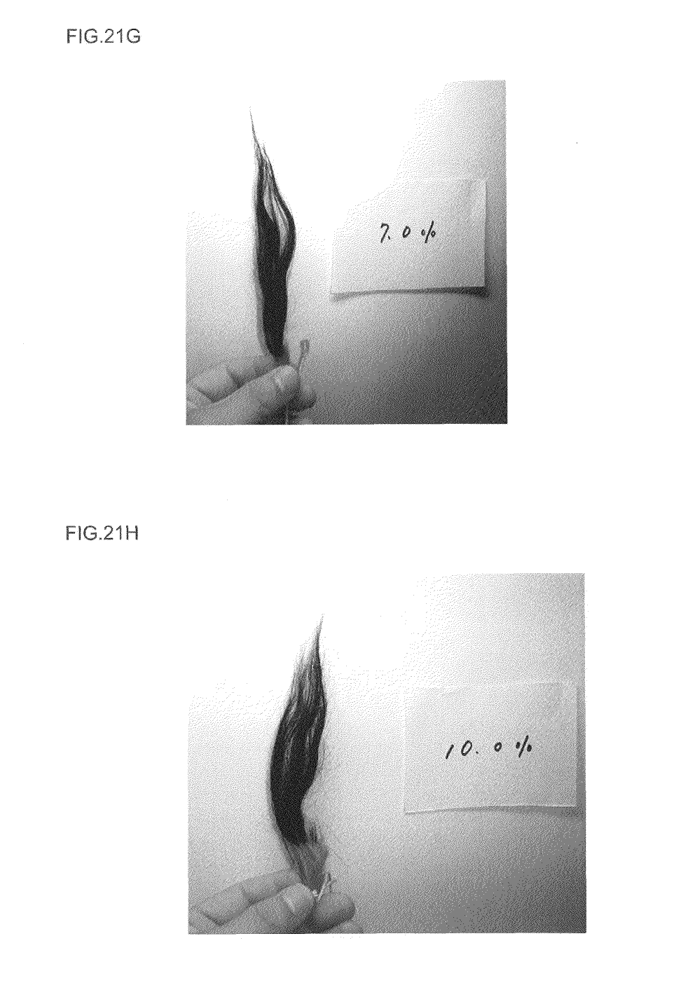

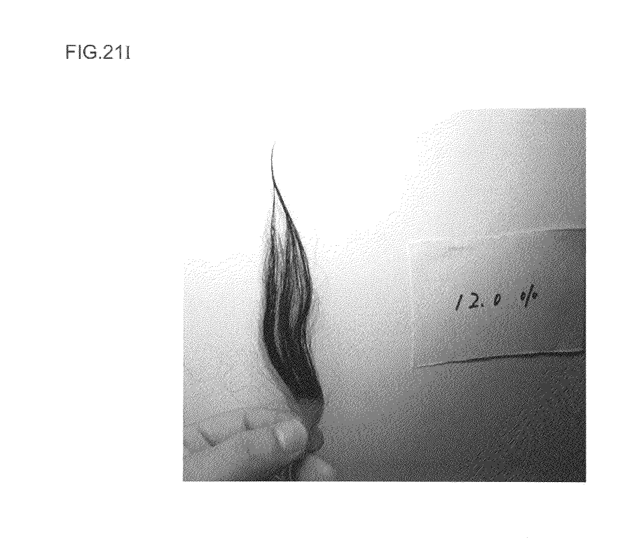

Photos of bundles of hair subjected to the processing with the hair processing agent compositions for dyeing hair of Comparative Example 5 and Examples 44 to 51 are shown in FIGS. 21A to 21I.

TABLE 11

| | Hair processing agent | | | | | | | | |
|---|---|---|---|---|---|---|---|---|---|
| | composition for dyeing hair | | | | | | | | |
| | HC-basic dye | Hair processing | | | | | | | |
| | BC Hair Color | agent composition | | Performance evaluation | | | | | |
| | Treatment BL | | Content | Bounce and | | | | | |
| | (g) | Kind | (g) | resilience | Rise | Stiffness | Sustainability | Dyeing result | Voluminousness |
| Example 44 | 100.0 | Sample A | 100.0 | Good | Good | Excellent | Good | Excellent | Good |
| Example 45 | 100.0 | Sample B | 100.0 | Good | Good | Excellent | Good | Excellent | Good |
| Example 46 | 100.0 | Sample C | 100.0 | Good | Excellent | Good | Good | Excellent | Excellent |
| Example 47 | 100.0 | Sample D | 100.0 | Excellent | Excellent | Good | Good | Excellent | Excellent |
| Example 48 | 100.0 | Sample E | 100.0 | Excellent | Excellent | Good | Good | Excellent | Excellent |
| Example 49 | 100.0 | Sample F | 100.0 | Excellent | Excellent | Good | Good | Good | Excellent |
| Example 50 | 100.0 | Sample G | 100.0 | Excellent | Excellent | Good | Good | Good | Excellent |
| Example 51 | 100.0 | Sample H | 100.0 | Excellent | Excellent | Good | Good | Fair | Excellent |
| Comp. Ex. 5 | 100.0 | — | — | Failed | Failed | Excellent | Good | Excellent | Failed |

### (5) Manufacturing of Hair Processing Agent Compositions of Sample I and Sample J

The hair processing agent compositions of Sample I and Sample J were prepared by blending the aforementioned materials to have the compositions shown in Table 12 [Content (mass %) of each material in Table 12 is a value based on the entire amount of the hair processing agent composition (entire amount of the hair processing agent composition as 100 mass %)]. They were obtained by firstly mixing silica particles with an oil-soluble dispersant in advance and, then, adding a surfactant and an aqueous solvent thereto to form an emulsion. Regarding the pH in the hair processing agent compositions, the Sample A had the pH of 5.5, and the Sample B had the pH of 9.0. The measurement of the pH values was performed by the use of a pH meter produced by Horiba, Ltd., by putting an electrode bar of the pH meter in each of the hair processing agent compositions prepared above (putting the electrode bar into the emulsion).

TABLE 12

| | Hair processing agent composition | | | | | | | |
|---|---|---|---|---|---|---|---|---|
| | | | | | Surfactant | | | |
| | | | | | Sodium polyoxy- | | | |
| | Silica particle | | Oil-soluble dispersant | | ethylene lauryl- | Aqueous solvent | | |
| | | Content | Cetanol | Lanoline alcohol | ether sulfate | Water | Triethanol amine | Total |
| | Product Name | (mass %) | (mass %) | (mass %) | (mass %) | (mass %) | (mass %) | (mass %) |
| Sample I | AEROSIL-R812 | 5.00 | 5.00 | 0.05 | 2.00 | 87.85 | 0.10 | 100.00 |
| Sample J | AEROSIL-R812 | 5.00 | 5.00 | 0.05 | 2.00 | 87.95 | 0.00 | 100.00 |

### (6) Hair Processing Agent Composition for Shampooing

### Example 52

By mixing 5 g of the hair processing agent composition of Sample I and 40 ml of “COCO NATURA Shampoo (trade name)” (Kikuboshi Corporation), the hair processing agent composition for shampooing of Example 52 was obtained (Table 13).

TABLE 13

| | Hair processing agent composition for shampooing | | | |
|---|---|---|---|---|
| | Hair processing | | | |
| | agent composition | | Shampoo agent | |
| | Kind | Amount (g) | Kind | Amount (ml) |
| Example 52 | Sample I | 5 | COCO NATURA | 40 |
| | | | shampoo | |
| | | | (Kikuboshi | |
| | | | Corporation) | |

(Shampoo and Hair Treatment)

In Usage Examples 1 and 2, shampooing and hair treatment were performed by the combinations shown in Table 14. First, prewashing of head hair was performed with water, and then a shampoo agent (hair processing agent composition for shampooing of Example 52) was applied to the head hair to shampoo the hair. After the head hair was rinsed with water subsequently, a hair treatment agent was applied to and allowed to penetrate into the head hair (to be applied evenly), and then the head hair was washed with water.

The evaluation results of the head hair after the shampoo and the hair treatment are shown in Table 14.

Also, the head hair after the hair treatment was evaluated for friction (Kishimi, squeaky feeling). In Usage Example 1, slight friction of the head hair was felt after the hair treatment (expressed as “fair” in the column of “friction” in Table 14). Usage Example 2 falls under the case where the hair treatment agent contains silicone. In Usage Example 2, no friction of the head hair was felt after the hair treatment (expressed as “excellent” in the column of “friction” in Table 14).

TABLE 14

| | | | Performance evaluation | | | | |
|---|---|---|---|---|---|---|---|
| | Shampoo | | Bounce and | | | | |
| | agent | Hair treatment agent | resilience | Rise | Stiffness | Sustainability | Friction |
| Usage | Hair processing agent | COCO NATURA treatment | Good | Excellent | Good | Excellent | Fair |
| Ex. 1 | composition for shampooing of | (Kikuboshi Corporation) | | | | | |
| | Example 52 | | | | | | |
| Usage | Hair processing agent | PANTENE PRO-V | Excellent | Excellent | Excellent | Excellent | Excellent |
| Ex. 2 | composition for shampooing of | Extra Damage Care | | | | | |
| | Example 52 | Treatment | | | | | |
| | | (P&G) | | | | | |

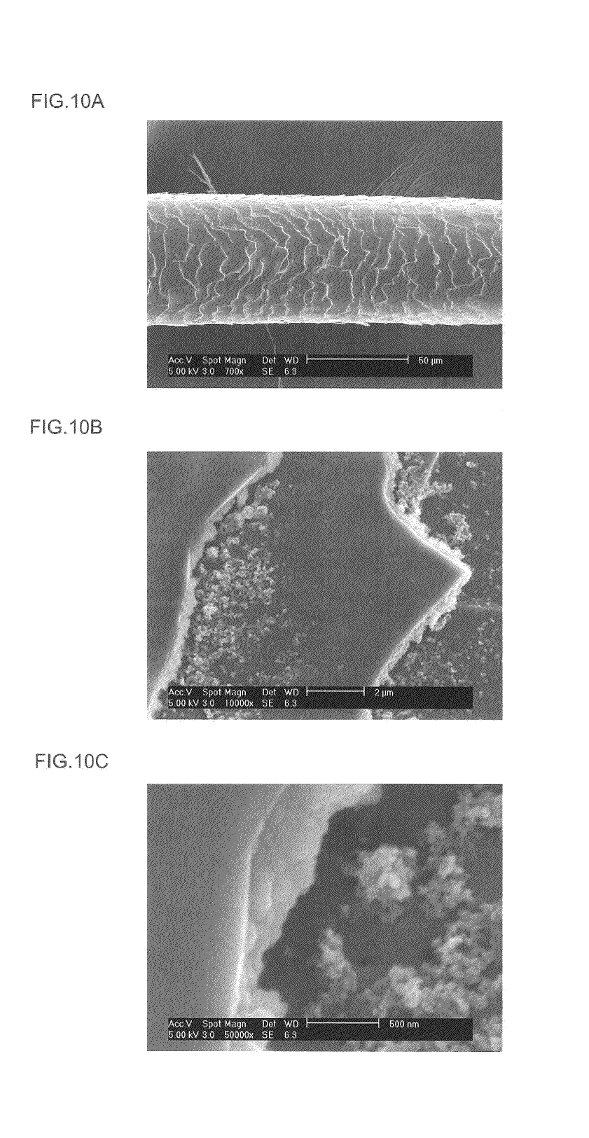

Regarding the head hair after shampoo and hair treatment of Usage Example 1 using the hair processing agent composition for shampooing of Example 52, there are shown the whole image (FIG. 10A), an enlarged image of cuticles (FIG. 10B), and an enlarged image of a cuticle edge portion (FIG. 10C). In the head hair of Usage Example 1 of Example 52, there was observed a state where silica particles stayed inside the gap between a cuticle edge portion exposed to the outside and an another cuticle covered with the cuticle edge portion of the outside cuticle and adjacently located on the tip side of the outside cuticle (FIGS. 10B and 10C). In addition, in the head hair of Usage Example 1 of Example 52, a few silica particles scattered on the surface in the vicinity of the cuticle edge portion.

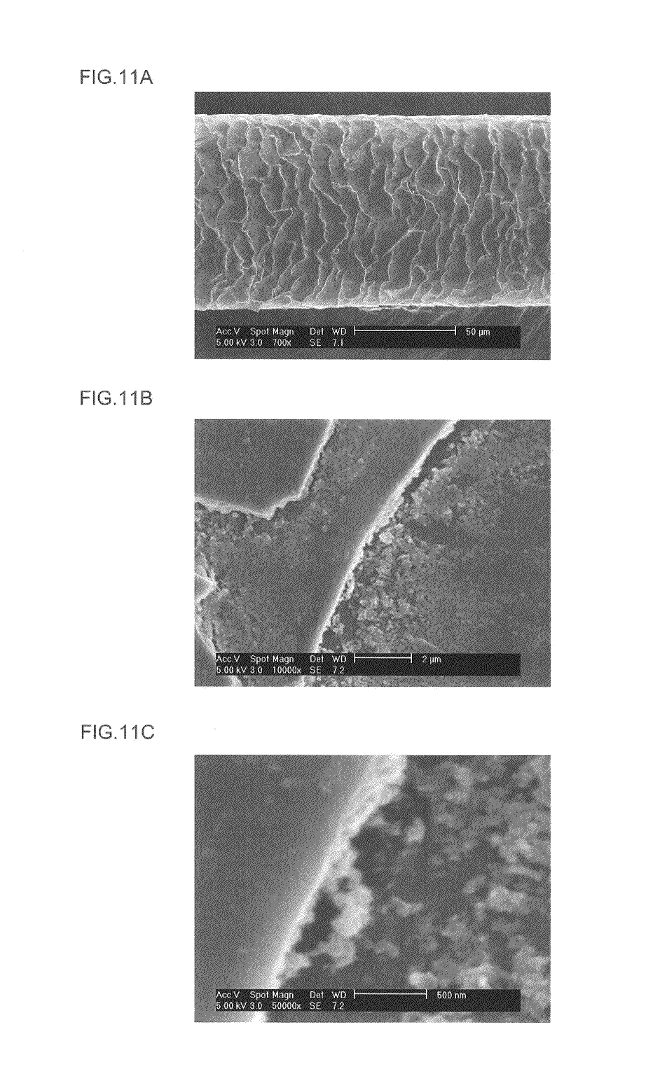

Regarding the head hair after shampoo and treatment of Usage Example 2 using the hair processing agent composition for shampooing of Example 52, there are shown the whole image (FIG. 11A), an enlarged image of cuticles (FIG. 11B), and an enlarged image of a cuticle edge portion (FIG. 11C). In the head hair of Usage Example 2 of Example 52, there was observed a state where silica particles stayed inside the gap between a cuticle edge portion exposed to the outside and an another adjacent cuticle covered with the cuticle edge portion and located on the tip side of the outside cuticle (FIGS. 11B and 11C).

In addition, in the head hair of Usage Example 2 of Example 52, a slightly larger amount of silica particles adhered to the surface in the vicinity of the edge portion of the cuticle. However, in comparison with the head hair of Comparative Example 1, where silica particles adhered to the surface of the cuticle in the state of having a bloom and emerging (in the state where the particles have emerged clearly on the cuticle surface) (FIG. 8B), the head hair of Usage Example 2 of Example 52 had a state where silica particles clung (stuck) closely to the surface of the cuticle (FIG. 11B). It was speculated that such a difference in the adhesion conditions of silica particles related to the “excellent” in “stiffness” of the head hair of Usage Example 2 of Example 52 (Table 14), while “stiffness” of the head hair of Comparative Example 1 was “failed” (Table 2).

### Examples 53 to 88, Comparative Examples 6 to 8

The hair processing agent compositions for shampooing of Examples 53 to 88 and Comparative Examples 6 to 8 were prepared by blending the aforementioned materials to have compositions shown in Tables 15 to 17.

Shampooing and hair treatment were performed by the combinations of hair processing agent compositions for shampooing of Examples 53 to 88 and Comparative Examples 6 to 8 with Pantene Pro-V Extra Damage Care Treatment (P&G). The head hair was firstly subjected to prewashing with water and, then, washed with applying a shampoo agent to the head hair (one of the hair processing agent compositions for shampooing of Examples 53 to 88 and Comparative Examples 6 to 8). After the head hair was subsequently rinsed with water, a hair treatment agent was applied to and allowed to penetrate into the head hair, the hair was washed with water.

The results are shown in Tables 15 to 17. Note that evaluation criteria of foaming are as follows.

### Evaluation Criteria of Foaming:

Excellent: much foamed

Good: foamed

Fair: somewhat foamed

Failed: not foamed

TABLE 15

| | Hair processing agent composition | | | | | | | |
|---|---|---|---|---|---|---|---|---|
| | for shampooing | | | | | | | |
| | | | Shampoo agent | | | | | |
| | Hair processing | | Higher alcohol base: | | | | | |
| | agent composition | | Pantene Extra Damage Hair | Performance evaluation | | | | |
| | | Content | Shampoo | | Bounce and | | | |
| | Kind | (g) | (g) | Foaming | resilience | Rise | Sustainability | Voluminousness |
| Example 53 | Sample E | 3 | 97 | Excellent | Failed | Failed | Failed | Failed |
| Example 54 | Sample H | 3 | 97 | Excellent | Fair | Fair | Fair | Fair |
| Example 55 | Sample E | 5 | 95 | Good | Fair | Fair | Fair | Fair |
| Example 56 | Sample H | 5 | 95 | Good | Good | Good | Good | Good |
| Example 57 | Sample E | 10 | 90 | Fair | Good | Good | Good | Good |
| Example 58 | Sample H | 10 | 90 | Fair | Excellent | Excellent | Excellent | Excellent |
| Example 59 | Sample A | 3 | 97 | Excellent | Failed | Failed | Failed | Failed |
| Example 60 | Sample A | 5 | 95 | Good | Failed | Failed | Failed | Failed |
| Example 61 | Sample A | 10 | 90 | Fair | Failed | Failed | Failed | Failed |
| Example 62 | Sample A | 15 | 85 | Failed | Failed | Failed | Failed | Failed |
| Example 63 | Sample E | 15 | 85 | Failed | Fair | Fair | Fair | Fair |
| Example 64 | Sample H | 15 | 85 | Failed | Excellent | Excellent | Excellent | Excellent |
| Comp. Ex. 6 | — | — | 100 | Excellent | Failed | Failed | Failed | Failed |

TABLE 16

| | Hair processing agent composition | | | | | | | |
|---|---|---|---|---|---|---|---|---|
| | for shampooing | | | | | | | |
| | Hair processing | | Shampoo agent | | | | | |
| | agent composition | | Soap base: | Performance evaluation | | | | |
| | | Content | COCO NATURA Shampoo | | Bounce and | | | |
| | Kind | (g) | (g) | Foaming | resilience | Rise | Sustainability | Voluminousness |
| Example 65 | Sample E | 3 | 97 | Excellent | Fair | Fair | Fair | Fair |
| Example 66 | Sample H | 3 | 97 | Excellent | Fair | Fair | Fair | Fair |
| Example 67 | Sample E | 5 | 95 | Good | Fair | Fair | Fair | Fair |
| Example 68 | Sample H | 5 | 95 | Good | Good | Good | Good | Good |
| Example 69 | Sample E | 10 | 90 | Fair | Good | Good | Good | Good |
| Example 70 | Sample H | 10 | 90 | Fair | Excellent | Excellent | Excellent | Excellent |
| Example 71 | Sample A | 3 | 97 | Excellent | Failed | Failed | Failed | Failed |
| Example 72 | Sample A | 5 | 95 | Good | Failed | Failed | Failed | Failed |
| Example 73 | Sample A | 10 | 90 | Fair | Failed | Failed | Failed | Failed |
| Example 74 | Sample A | 15 | 85 | Failed | Failed | Failed | Failed | Failed |
| Example 75 | Sample E | 15 | 85 | Failed | Good | Good | Good | Good |
| Example 76 | Sample H | 15 | 85 | Failed | Excellent | Excellent | Excellent | Excellent |
| Comp. Ex. 7 | — | — | 100 | Excellent | Failed | Failed | Failed | Failed |

TABLE 17

| | Hair processing agent composition | | | | | | | |
|---|---|---|---|---|---|---|---|---|
| | for shampooing | | | | | | | |
| | Hair processing | | Shampoo agent | | | | | |
| | agent composition | | Amino acid base: | Performance evaluation | | | | |
| | | Content | Lasana Shampoo | | Bounce and | | | |
| | Kind | (g) | (g) | Foaming | resilience | Rise | Sustainability | Voluminousness |
| Example 77 | Sample E | 3 | 97 | Excellent | Fair | Fair | Fair | Fair |
| Example 78 | Sample H | 3 | 97 | Excellent | Fair | Fair | Fair | Fair |
| Example 79 | Sample E | 5 | 95 | Good | Fair | Fair | Fair | Fair |
| Example 80 | Sample H | 5 | 95 | Good | Good | Good | Good | Good |
| Example 81 | Sample E | 10 | 90 | Fair | Good | Good | Good | Good |
| Example 82 | Sample H | 10 | 90 | Fair | Excellent | Excellent | Excellent | Excellent |
| Example 83 | Sample A | 3 | 97 | Excellent | Failed | Failed | Failed | Failed |
| Example 84 | Sample A | 5 | 95 | Good | Failed | Failed | Failed | Failed |
| Example 85 | Sample A | 10 | 90 | Fair | Failed | Failed | Failed | Failed |
| Example 86 | Sample A | 15 | 85 | Failed | Failed | Failed | Failed | Failed |
| Example 87 | Sample E | 15 | 85 | Failed | Good | Good | Good | Good |
| Example 88 | Sample H | 15 | 85 | Failed | Excellent | Excellent | Excellent | Excellent |
| Comp. Ex. 8 | — | — | 100 | Excellent | Failed | Failed | Failed | Failed |

### (7) Hair Processing Agent Composition for Hair Treatment

### Examples 89 and 90

A hair processing agent composition for hair treatment was obtained by mixing 5 g of hair processing agent composition of the Sample I with 40 ml of “COCO NATURA treatment (product name)” (Kikuboshi Corporation) in Example 89, and a hair processing agent composition for hair treatment was obtained by mixing 5 g of hair processing agent composition of the Sample I with 40 ml of “Pantene Pro-V Extra Damage Care Treatment (product name)” (P&G) in Example 90 (Table 18).

TABLE 18

| | Hair processing agent composition for hair treatment | | | |
|---|---|---|---|---|
| | Hair processing | | | |
| | agent composition | | Hair treatment agent | |
| | Kind | Amount (g) | Kind | Amount (ml) |
| Example 89 | Sample I | 5 | COCO NATURA | 40 |
| | | | treatment | |
| | | | (Kikuboshi | |
| | | | Corporation) | |
| Example 90 | Sample I | 5 | Pantene Pro-V | 40 |
| | | | Extra Damage | |
| | | | Care Treatment | |
| | | | (P&G) | |

(Shampoo and Hair Treatment)

In Usage Examples 3 and 4, shampooing and hair treatment were performed with the combinations shown in Table 19. First, prewashing of head hair was performed with water, and then a shampoo agent was applied to the head hair to shampoo the hair. After the head hair was rinsed with water subsequently, a hair treatment agent (hair processing agent composition for hair treatment of Example 89 or Example 90) was applied to and allowed to penetrate into the head hair, and then the head hair was washed with water.

The evaluation results of the head hair after the shampooing and the hair treatment are shown in Table 19.

Also, the head hair after the hair treatment was evaluated for friction. In Usage Examples 3 and 4, no friction of the head hair was felt after the hair treatment (expressed as “excellent” in the column of “friction” in Table 19).

TABLE 19

| | | | Performance evaluation | | | | |
|---|---|---|---|---|---|---|---|
| | Shampoo | | Bounce and | | | | |
| | agent | Hair treatment agent | resilience | Rise | Stiffness | Sustainability | Friction |
| Usage | COCO NATURA shampoo | Hair processing agent | Good | Good | Good | Excellent | Excellent |
| Example 3 | (Kikuboshi Corporation) | composition for hair treatment | | | | | |
| | | of Example 89 | | | | | |
| Usage | COCO NATURA shampoo | Hair processing agent | Excellent | Excellent | Good | Excellent | Excellent |
| Example 4 | (Kikuboshi Corporation) | composition for hair treatment | | | | | |
| | | of Example 90 | | | | | |

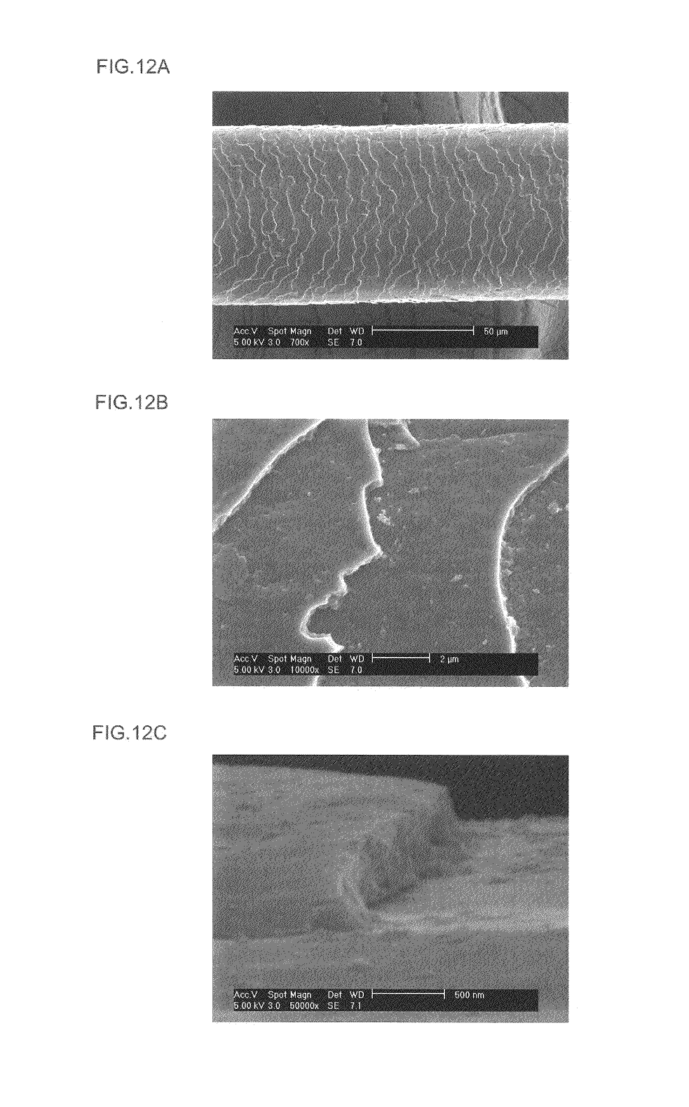

Regarding the head hair after shampoo and treatment using the hair processing agent composition for hair treatment of Example 89 (Usage Example 3), there are shown the whole image (FIG. 12A), an enlarged image of cuticles (FIG. 12B), and an enlarged image of a cuticle edge portion (FIG. 12C). In the head hair of Example 89 (Usage Example 3), there was observed a state where silica particles stayed inside the gap between a cuticle edge portion exposed to the outside and an inside cuticle covered with this and located on the tip side (FIGS. 12B and 12C). In addition, in the head hair of Example 89 (Usage Example 3), almost no silica particle was observed on the surface of the cuticle.

### Examples 91 to 122, Comparative Examples 9 and 10

The hair processing agent compositions for shampooing of Examples 91 to 122 and Comparative Examples 9 and 10 were prepared by blending the aforementioned materials to have compositions shown in Tables 20 and 21.

The hair processing agent compositions for hair treatment of Examples 91 to 106 and Comparative Example 9 were combined with Pantene Extra Damage Hair Shampoo (P&G) to perform shampoo and hair treatment. First, prewashing of head hair was performed with water, and then a shampoo agent was applied to the head hair to shampoo the hair. After the head hair was rinsed with water subsequently, a hair treatment agent (one of the hair processing agent compositions for shampooing of Examples 91 to 122 and Comparative Example 9) was applied to and allowed to penetrate into the head hair, and then the head hair was washed with water.

The hair processing agent compositions for hair treatment of Examples 107 to 122 and Comparative Example 10 were combined with COCO NATURA Shampoo (Kikuboshi Corporation) to perform shampoo and hair treatment. First, prewashing of head hair was performed with water, and then a shampoo agent was applied to the head hair to shampoo the hair. After the head hair was rinsed with water subsequently, a hair treatment agent (one of the hair processing agent compositions for shampooing of Examples 107 to 122 and Comparative Example 10) was applied to and allowed to penetrate into the head hair, and then the head hair was washed with water.

The results are shown in Tables 20 and 21. Note that the evaluation criteria for conditioning are as follows:

### Evaluation for Conditioning:

Excellent: Head hair becomes very smooth.

Good: Head hair becomes smooth.

Fair: Head hair becomes slightly smooth.

Failed: Head hair does not become smooth.

TABLE 20

| | Hair processing agent composition | | | | | | | |
|---|---|---|---|---|---|---|---|---|
| | for shampooing | | | | | | | |
| | | | Hair treatment | | | | | |
| | | | agent | | | | | |
| | | | Cation base: | | | | | |
| | Hair processing | | Pantene Extra | | | | | |
| | agent composition | | Damage Hair | Performance evaluation | | | | |
| | | Content | Treatment | | Bounce and | | | |
| | Kind | (g) | (g) | Conditioning | resilience | Rise | Sustainability | Voluminousness |
| Example 91 | Sample A | 30 | 70 | Excellent | Fair | Fair | Fair | Fair |
| Example 92 | Sample D | 30 | 70 | Excellent | Good | Good | Good | Good |
| Example 93 | Sample E | 30 | 70 | Excellent | Excellent | Excellent | Excellent | Excellent |
| Example 94 | Sample H | 30 | 70 | Excellent | Excellent | Excellent | Excellent | Excellent |
| Example 95 | Sample A | 40 | 60 | Good | Fair | Fair | Fair | Fair |
| Example 96 | Sample D | 40 | 60 | Good | Excellent | Excellent | Excellent | Excellent |
| Example 97 | Sample E | 40 | 60 | Good | Excellent | Excellent | Excellent | Excellent |
| Example 98 | Sample H | 40 | 60 | Good | Excellent | Excellent | Excellent | Excellent |
| Example 99 | Sample A | 50 | 50 | Fair | Fair | Fair | Fair | Fair |
| Example 100 | Sample D | 50 | 50 | Fair | Excellent | Excellent | Excellent | Excellent |
| Example 101 | Sample E | 50 | 50 | Fair | Good | Good | Good | Good |
| Example 102 | Sample H | 50 | 50 | Fair | Excellent | Excellent | Excellent | Excellent |
| Example 103 | Sample A | 60 | 40 | Failed | Fair | Fair | Fair | Fair |
| Example 104 | Sample D | 60 | 40 | Failed | Excellent | Excellent | Excellent | Excellent |
| Example 105 | Sample E | 60 | 40 | Failed | Excellent | Excellent | Excellent | Excellent |
| Example 106 | Sample H | 60 | 40 | Failed | Excellent | Excellent | Excellent | Excellent |
| Comp. Ex. 9 | — | — | 100 | Excellent | Failed | Failed | Failed | Failed |

TABLE 21

| | Hair processing agent composition | | | | | | | |
|---|---|---|---|---|---|---|---|---|
| | for shampooing | | | | | | | |
| | Hair processing | | Non-cation base: | | | | | |
| | agent composition | | COCO NATURA | Performance evaluation | | | | |
| | | Content | treatment | | Bounce and | | | |
| | Kind | (g) | (g) | Conditioning | resilience | Rise | Sustainability | Voluminousness |
| Example 107 | Sample A | 30 | 70 | Excellent | Fair | Fair | Fair | Fair |
| Example 108 | Sample D | 30 | 70 | Excellent | Good | Good | Good | Good |
| Example 109 | Sample E | 30 | 70 | Excellent | Excellent | Excellent | Excellent | Excellent |
| Example 110 | Sample H | 30 | 70 | Excellent | Excellent | Excellent | Excellent | Excellent |
| Example 111 | Sample A | 40 | 60 | Good | Fair | Fair | Fair | Fair |
| Example 112 | Sample D | 40 | 60 | Good | Excellent | Excellent | Excellent | Excellent |
| Example 113 | Sample E | 40 | 60 | Good | Excellent | Excellent | Excellent | Excellent |
| Example 114 | Sample H | 40 | 60 | Good | Excellent | Excellent | Excellent | Excellent |
| Example 115 | Sample A | 50 | 50 | Fair | Fair | Fair | Fair | Fair |
| Example 116 | Sample D | 50 | 50 | Fair | Excellent | Excellent | Excellent | Excellent |
| Example 117 | Sample E | 50 | 50 | Fair | Good | Good | Good | Good |
| Example 118 | Sample H | 50 | 50 | Fair | Excellent | Excellent | Excellent | Excellent |
| Example 119 | Sample A | 60 | 40 | Failed | Fair | Fair | Fair | Fair |
| Example 120 | Sample D | 60 | 40 | Failed | Excellent | Excellent | Excellent | Excellent |
| Example 121 | Sample E | 60 | 40 | Failed | Excellent | Excellent | Excellent | Excellent |
| Example 122 | Sample H | 60 | 40 | Failed | Excellent | Excellent | Excellent | Excellent |
| Comp. Ex. 10 | — | — | 100 | Excellent | Failed | Failed | Failed | Failed |

### (8) Hair Processing Agent Composition for Perming

### Example 123

In Example 123, 5 g of hair processing agent composition of the Sample J and 40 ml of the first agent of “Cream S. Design Lipid (trade name)” (Lebel Cosmetics) were mixed to be used as the first agent of the hair processing agent composition for perming, and 40 ml of the second agent of “Cream S. Design Lipid (trade name)” (Lebel Cosmetics) was used as the second agent of the hair processing agent composition for perming (Table 22).

### Example 124

In Example 124, 5 g of hair processing agent composition of the Sample J and 40 ml of the first agent of “Cream CYS (trade name)” (Lebel Cosmetics) were mixed to be used as the first agent of the hair processing agent composition for perming, and 40 ml of the second agent of “Cream CYS (trade name)” (Lebel Cosmetics) was used as the second agent of the hair processing agent composition for perming (Table 22).

### Example 125

In Example 125, 40 ml of the first agent of “Cream S. Design Lipid (trade name)” (Lebel Cosmetics) was used as the first agent of the hair processing agent composition for perming, and 5 g of hair processing agent composition of the Sample J and 40 ml of the second agent of “Cream S. Design Lipid (trade name)” (Lebel Cosmetics) were mixed to be used as the second agent of the hair processing agent composition for perming (Table 23).

### Example 126

In Example 126, 40 ml of the first agent of “Cream CYS (trade name)” (Lebel Cosmetics) was used as the first agent of the hair processing agent composition for perming, and 5 g of hair processing agent composition of the sample J and 40 ml of the second agent of “Cream CYS (trade name)” (Lebel Cosmetics) were mixed to be used as the second agent of the hair processing agent composition for perming (Table 23).

(Perming)

Head hair was shampooed, and water of the head hair was removed with a towel. Next, the first agent of a hair processing agent composition for perming was applied to the head hair, and the head hair was wound on rods and heated for 20 minutes. Next, after the test curl was confirmed, the second agent of the hair processing agent composition for perming was applied to the head hair and let stand for 5 minutes. Then, the rods were removed from the head hair, and, after the head hair was rinsed with lukewarm water, a rinse was applied to the head hair for finishing.

The evaluation results of the head hair after perming are shown in Table 22 (Examples 123 and 124) and Table 23 (Examples 125 and 126).

By any of the hair processing agent compositions for perming of Examples 123 to 126, no stimulus to the scalp was felt, which is felt upon using a conventional perming agent (expressed as “none” in the column of “stimulus to scalp” in Tables 22 and 23).

In Examples 123 and 125, well perming as in the general use of “Cream S. DESIGN LIPID” (Lebel Cosmetics) was possible (expressed as “excellent” in the column of “perm” in Tables 22 and 23). In addition, in Examples 124 and 126, well perming as in the general use of “CREAM CYS” (Lebel Cosmetics) was possible (expressed as “excellent” in the column of “perm” in Tables 22 and 23).

TABLE 22

| | Hair processing agent composition for perming | | | | | |
|---|---|---|---|---|---|---|
| | First agent | | | | Second agent | |
| | Hair | | Perm | | Perm | |
| | processing agent | | solution | | solution | |
| | composition | | (First agent) | | (Second agent) | |
| | | Use | | Use | | Use |
| | | amount | | amount | | amount |
| | Kind | (g) | Kind | (ml) | Kind | (ml) |
| Example | Sample J | 5 | Cream S. | 40 | Cream S. | 40 |
| 123 | | | DESIGN LIPID | | DESIGN LIPID | |
| | | | First agent | | Second agent | |
| | | | (Lebel Cosmetics) | | (Lebel Cosmetics) | |
| Example | Sample J | 5 | CREAM CYS | 40 | CREAM CYS | 40 |
| 124 | | | First agent | | Second agent | |
| | | | (Lebel | | (Lebel | |
| | | | Cosmetics) | | Cosmetics) | |

| | Performance evaluation | | | | | |
|---|---|---|---|---|---|---|
| | Bounce | | | | | |
| | and | | | | | Stimulus to |
| | resilience | Rise | Stiffness | Sustainability | Perm | scalp |
| Example | Good | Excellent | Good | Excellent | Excellent | None |
| 123 | | | | | | |
| Example | Good | Excellent | Good | Excellent | Excellent | None |
| 124 | | | | | | |

TABLE 23

| | Hair processing agent composition for perming | | | | | | | | | | | |
|---|---|---|---|---|---|---|---|---|---|---|---|---|
| | First agent | | Second agent | | | | | | | | | |
| | Perm | | Hair | | Perm | | | | | | | |
| | solution | | processing agent | | solution | | | | | | | |
| | (First agent) | | composition | | (Second agent) | | Performance Evaluation | | | | | |
| | | Use | | Use | | Use | Bounce | | | | | Stimulus |
| | | amount | | amount | | amount | and | | Stiff- | Sustain- | | to |
| | Kind | (ml) | Kind | (g) | Kind | (ml) | resilience | Rise | ness | ability | Perm | scalp |
| Example | Cream S. | 40 | Sample J | 5 | Cream S. | 40 | Excellent | Excellent | Good | Excellent | Excellent | None |
| 125 | DESIGN | | | | DESIGN | | | | | | | |
| | LIPID | | | | LIPID | | | | | | | |
| | First agent | | | | Second | | | | | | | |
| | (Lebel | | | | agent | | | | | | | |
| | Cosmetics) | | | | (Lebel | | | | | | | |
| | | | | | Cosmetics) | | | | | | | |
| Example | CREAM | 40 | Sample J | 5 | CREAM | 40 | Good | Excellent | Good | Excellent | Excellent | None |
| 126 | CYS | | | | CYS | | | | | | | |
| | First agent | | | | Second | | | | | | | |
| | (Lebel | | | | agent | | | | | | | |
| | Cosmetics) | | | | (Lebel | | | | | | | |
| | | | | | Cosmetics) | | | | | | | |

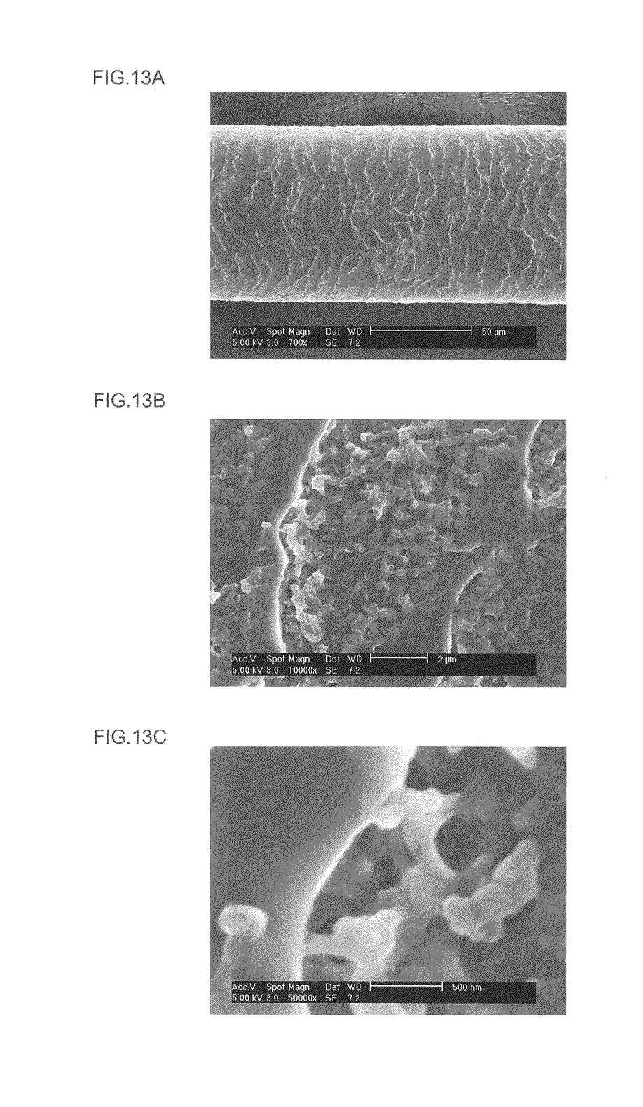

Regarding the head hair after perming using the hair processing agent composition for perming of Example 123, there are shown the whole image (FIG. 13A), an enlarged image of cuticles (FIG. 13B), and an enlarged image of a cuticle edge portion (FIG. 13C). In the head hair of Example 123, there was observed a state where silica particles stayed inside the gap between a cuticle edge portion exposed to the outside and a cuticle covered with the cuticle edge portion, adjacently located on the tip side of the outside cuticle (FIGS. 13B and 13C).

In addition, in the head hair of Example 123, a slightly larger amount of silica particles adhered to the surface in the vicinity of the edge portion of the cuticle. However, in comparison with the head hair of Comparative Example 1, where silica particles adhered to the surface of the cuticle in the state of having a bloom and emerging (FIG. 8B), the head hair of Example 123 had a state where silica particles clung closely to the surface of the cuticle (FIG. 13B). It was speculated that such a difference in the adhesion conditions of silica particles related to the “good” in “stiffness” of the head hair of Example 123 (Table 22), while “stiffness” of the head hair of Comparative Example 1 was “failed” (Table 2).

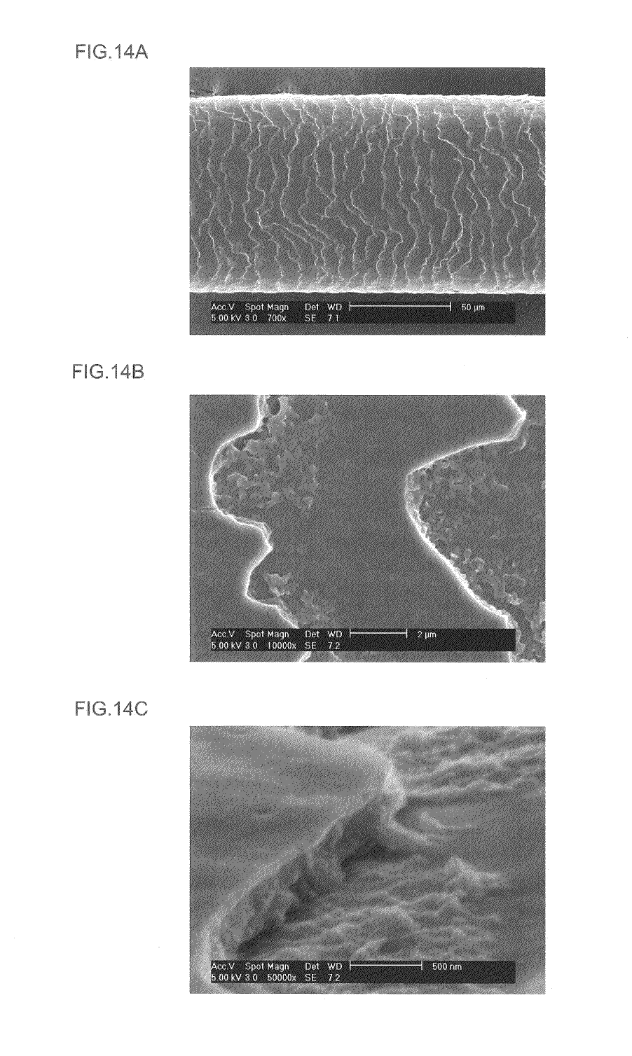

Regarding the head hair after perming using the hair processing agent composition for perming of Example 124, there are shown the whole image (FIG. 14A), an enlarged image of cuticles (FIG. 14B), and an enlarged image of a cuticle edge portion (FIG. 14C). In the head hair of Example 124, there was observed a state where silica particles stayed inside the gap between a cuticle edge portion exposed to the outside and an adjacent cuticle covered with the cuticle edge portion and located on the tip side of the outside cuticle (FIGS. 14B and 14C).

In addition, in the head hair of Example 124, silica particles slightly adhered to the surface in the vicinity of the cuticle edge portion. However, in comparison with the head hair of Comparative Example 1, where silica particles adhered to the surface of the cuticle in the state of having a bloom and emerging (FIG. 8B), the head hair of Example 124 had a state where silica particles clung closely to the surface of the cuticle (FIG. 14B). It was speculated that such a difference in the adhesion conditions of silica particles related to the “good” in “stiffness” of the head hair of Example 124 (Table 22), while “stiffness” of the head hair of Comparative Example 1 was “failed” (Table 2).

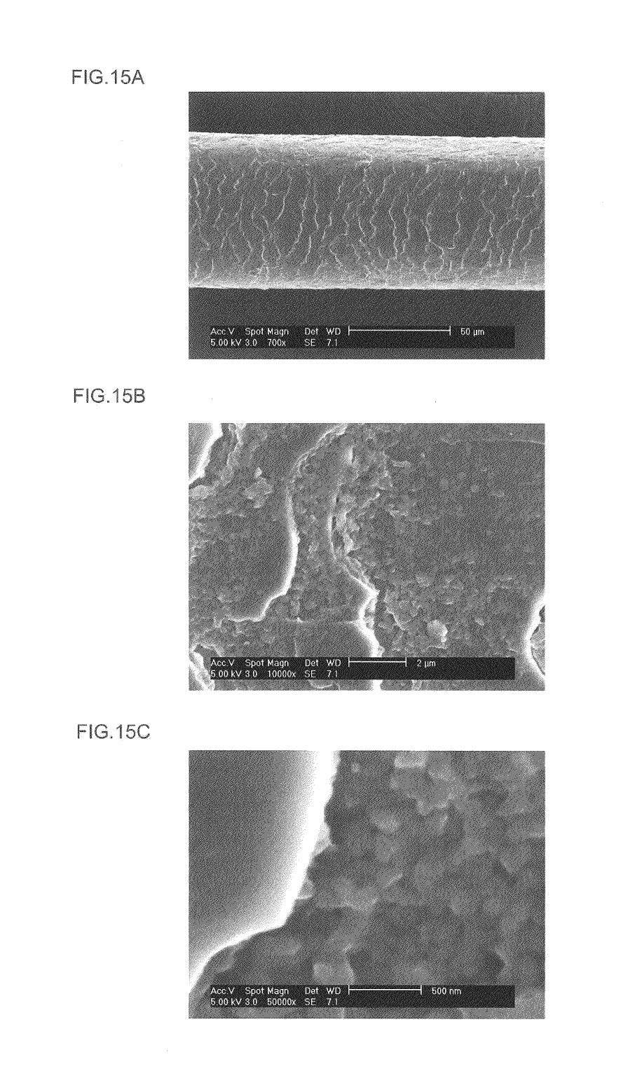

Regarding the head hair after perming using the hair processing agent composition for perming of Example 125, there are shown the whole image (FIG. 15A), an enlarged image of cuticles (FIG. 15B), and an enlarged image of a cuticle edge portion (FIG. 15C). In the head hair of Example 125, there was observed a state where silica particles stayed inside the gap between a cuticle edge portion exposed to the outside and an adjacent cuticle covered with the cuticle edge portion and located on the tip side of the outside cuticle (FIGS. 15B and 15C).

In addition, in the head hair of Example 125, a slightly larger amount of silica particles adhered to the surface in the vicinity of the edge portion of the cuticle. However, in comparison with the head hair of Comparative Example 1, where silica particles adhered to the surface of the cuticle in the state of having a bloom and emerging (FIG. 8B), the head hair of Example 125 had a state where silica particles clung closely to the surface of the cuticle (FIG. 15B). It was speculated that such a difference in the adhesion conditions of silica particles related to the “good” in “stiffness” of the head hair of Example 125 (Table 23), while “stiffness” of the head hair of Comparative Example 1 was “failed” (Table 2).

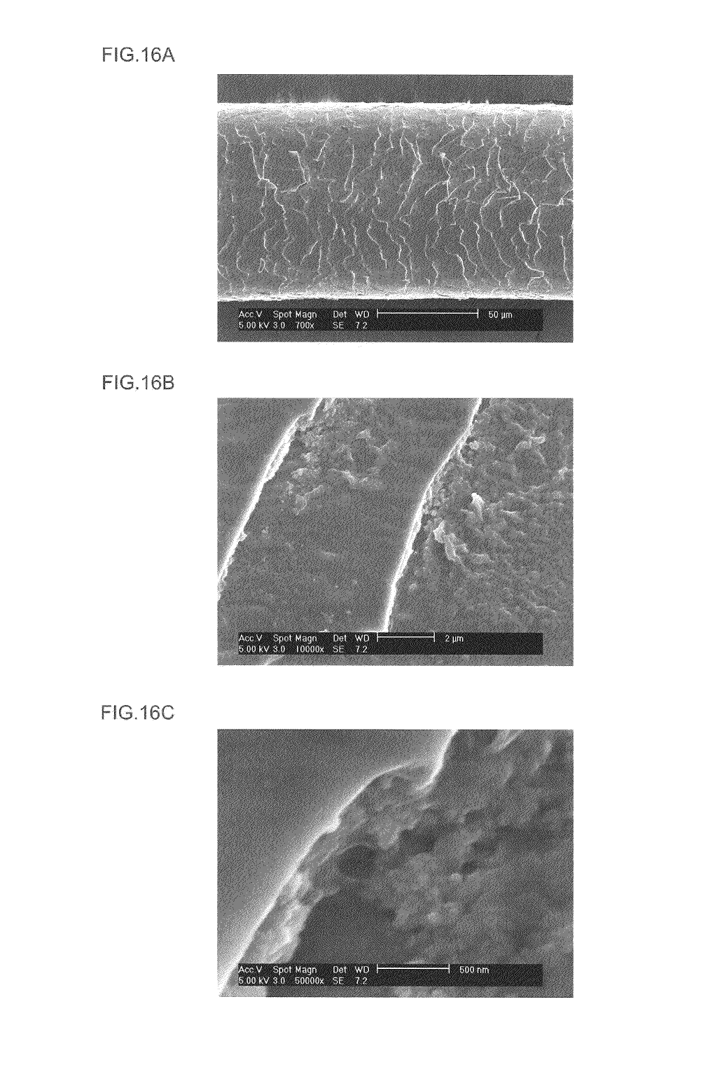

Regarding the head hair after perming using the hair processing agent composition for perming of Example 126, there are shown the whole image (FIG. 16A), an enlarged image of cuticles (FIG. 16B), and an enlarged image of a cuticle edge portion (FIG. 16C). In the head hair of Example 126, there was observed a state where silica particles stayed inside the gap between a cuticle edge portion exposed to the outside and an another cuticle covered with the cuticle edge portion and adjacently located on the tip side of the outside cuticle (FIGS. 16B and 16C).

In addition, in the head hair of Example 126, silica particles slightly adhered to the surface in the vicinity of the cuticle edge portion. However, in comparison with the head hair of Comparative Example 1, where silica particles adhered to the surface of the cuticle in the state of having a bloom and emerging (FIG. 8B), the head hair of Example 126 had a state where silica particles clung closely to the surface of the cuticle (FIG. 16B).

It was speculated that such a difference in the adhesion conditions of silica particles related to the “good” in “stiffness” of the head hair of Example 126 (Table 23), while “stiffness” of the head hair of Comparative Example 1 was “failed” (Table 2).

### Examples 127 to 134, Comparative Example 11

Hair processing agent compositions for perming of Examples 127 to 134 and Comparative Example 11 were prepared by blending the aforementioned materials to have the compositions shown in Table 24.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the hair (the bundle of hair) was washed with water, and then the second agent was applied to the hair (the bundle of hair) and let stand for 15 minutes. Subsequently, the hair (the bundle of hair) was washed with water and then dried.

Regarding “perm”, “excellent” was given to the case where the bundle of hair could be curled as in the general use of “Stealth TA Normal to Light Damage Hair” (Number Three Inc.). “Good” was given to the case where the curling condition was slightly weak in comparison with the curl in the general use of “Stealth TA Normal to Light Damage Hair” (Number Three Inc.). “Fair” was given to the case where the curling condition was weak in comparison with the curl in the general use of “Stealth TA Normal to Light Damage Hair” (Number Three Inc.). The results are shown in Table 24.

TABLE 24

| | Hair processing agent composition for perming | | | | | | | | | |
|---|---|---|---|---|---|---|---|---|---|---|
| | First agent | | | Second agent | | | | | | |
| | | | Perm | Perm | | | | | | |
| | | | solution | solution | | | | | | |
| | | | (First agent) | (Second agent) | | | | | | |
| | | | Stealth | Stealth | | | | | | |
| | Hair | | TA Normal | TA Normal | | | | | | |
| | processing agent | | to Light | to Light | Performance evaluation | | | | | |
| | composition | | Damage | Damage | Bounce | | | | | |
| | | Content | Hair | Hair | and | | | Sustain- | | Volumi- |
| | Kind | (g) | (g) | (g) | resilience | Rise | Stiffness | ability | Perm | nousness |
| Example 127 | Sample A | 50 | 50 | 100 | Good | Excellent | Excellent | Good | Excellent | Excellent |
| Example 128 | Sample B | 50 | 50 | 100 | Good | Excellent | Excellent | Good | Excellent | Excellent |
| Example 129 | Sample C | 50 | 50 | 100 | Excellent | Excellent | Good | Good | Excellent | Excellent |
| Example 130 | Sample D | 50 | 50 | 100 | Excellent | Excellent | Good | Good | Excellent | Excellent |
| Example 131 | Sample E | 50 | 50 | 100 | Excellent | Excellent | Good | Good | Excellent | Excellent |
| Example 132 | Sample F | 50 | 50 | 100 | Excellent | Excellent | Fair | Good | Excellent | Excellent |
| Example 133 | Sample G | 50 | 50 | 100 | Excellent | Excellent | Fair | Good | Good | Excellent |
| Example 134 | Sample H | 50 | 50 | 100 | Excellent | Excellent | Fair | Good | Fair | Excellent |
| Comp. Ex. 11 | — | — | 100 | 100 | Failed | Failed | Excellent | Good | Excellent | Fair |

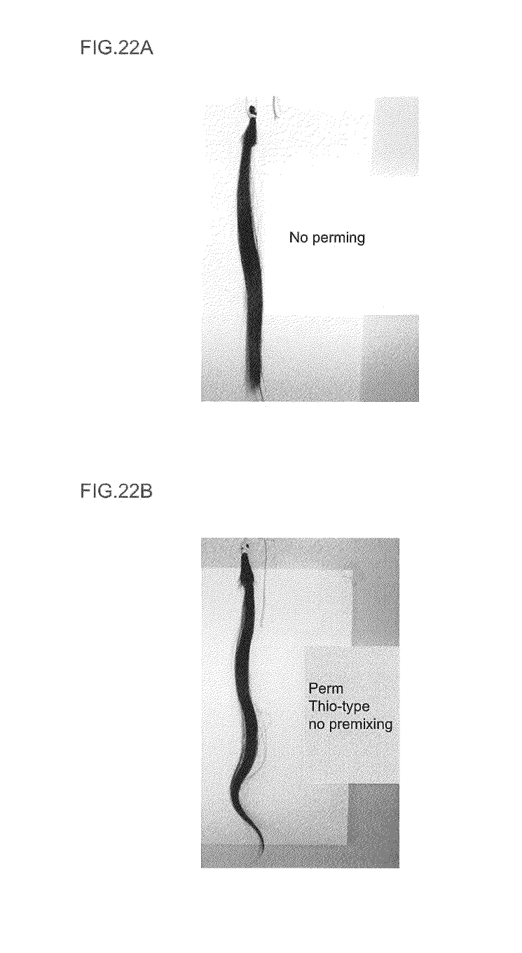

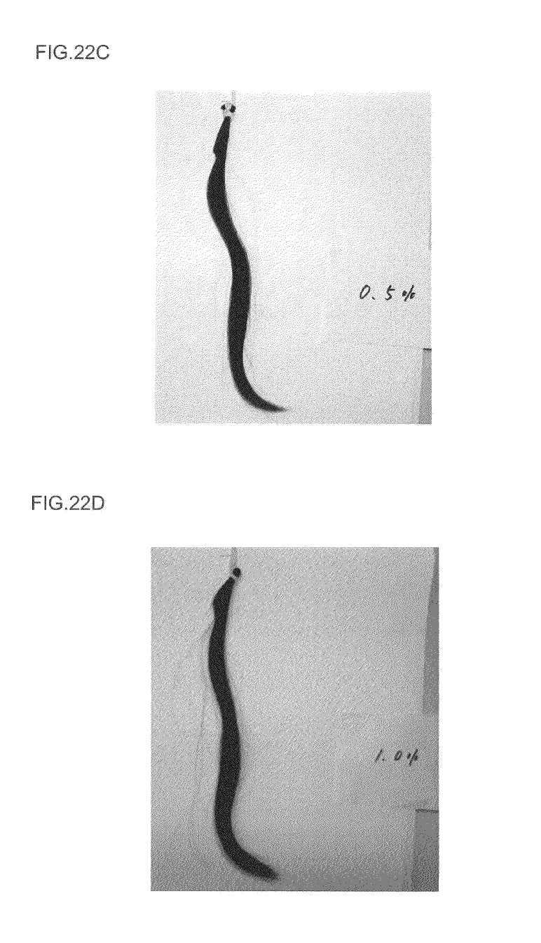

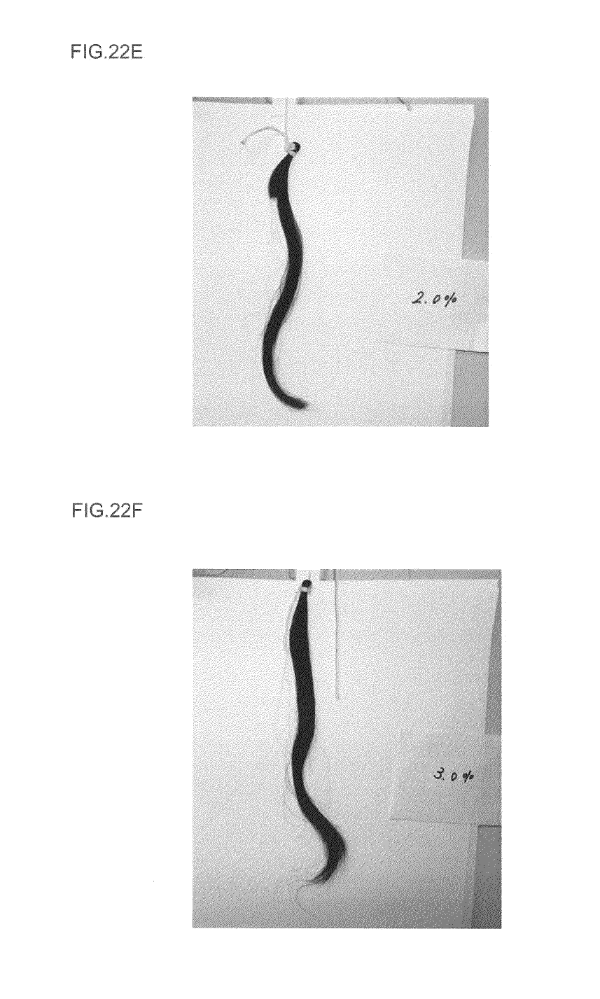

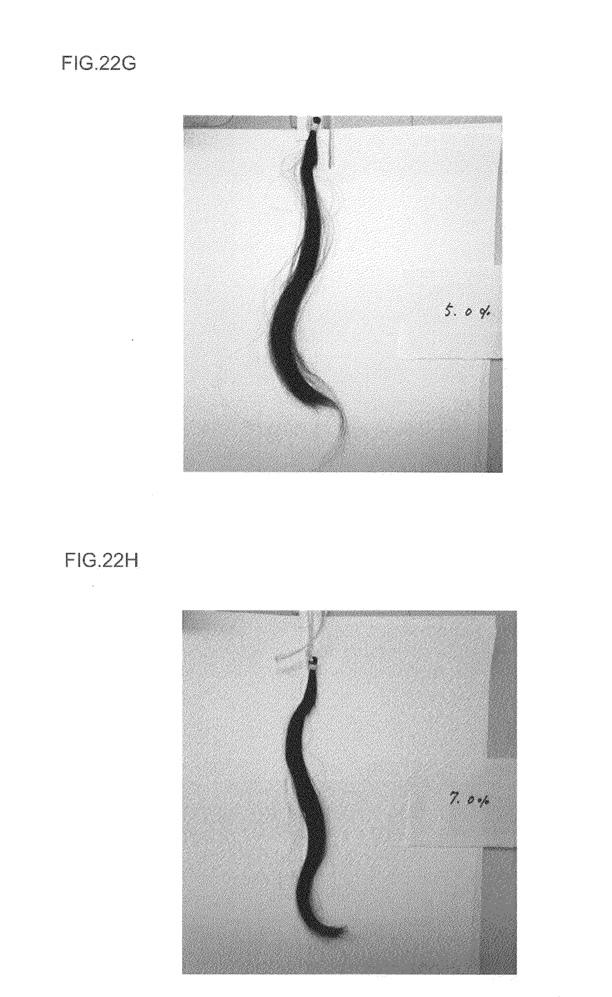

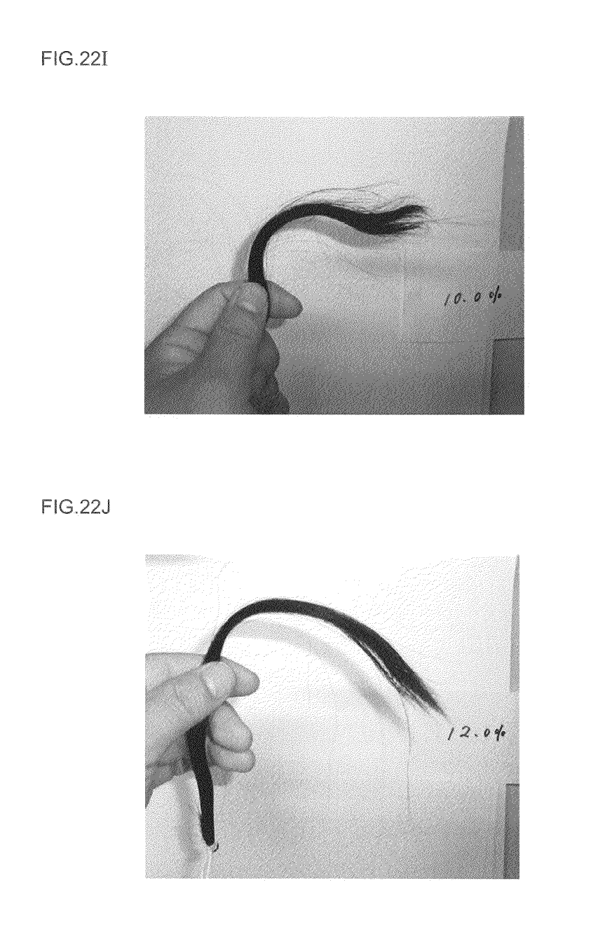

A photo of an unprocessed bundle of hair is shown in FIG. 22A, and photos of bundles of hair subjected to the processing with the hair processing agent compositions for perming of Comparative Example 11 and Examples 127 to 134 are shown in FIGS. 22B to 22J.

### Examples 135 to 142, Comparative Example 12

The hair processing agent compositions for perming of Examples 135 to 142 and Comparative Example 12 were prepared by blending the aforementioned materials to have compositions shown in Table 25.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the hair (the bundle of hair) was washed with water, and the second agent was applied to the hair (the bundle of hair) and let stand for 15 minutes. Subsequently, the hair (the bundle of hair) was washed with water and then dried.

Regarding “perm”, “excellent” was given to the case where the bundle of hair could be curled as in the general use of “Stealth CYS CY damage level 4 to 5” (Number Three Inc.). “Good” was given to the case where the curling condition was slightly weak in comparison with the curl in the general use of “Stealth CYS CY damage level 4 to 5” (Number Three Inc.). “Fair” was given to the case where the curling condition was weak in comparison with the curl in the general use of “Stealth CYS CY damage level 4 to 5” (Number Three Inc.). The results are shown in Table 25.

TABLE 25

| | Hair processing agent composition for perming | | | | | | | | | |
|---|---|---|---|---|---|---|---|---|---|---|
| | | | | Second agent | | | | | | |
| | First agent | | | Perm | | | | | | |
| | | | Perm | solution | | | | | | |
| | | | solution | (Second | | | | | | |
| | Hair processing | | (First agent) | agent) | Performance evaluation | | | | | |
| | agent composition | | Stealth CYS | Stealth CYS | Bounce | | | | | |
| | | Content | CY | CY | and | | | | | |
| | Kind | (g) | (g) | (g) | resilience | Rise | Stiffness | Sustainability | Perm | Voluminousness |
| Example 135 | Sample A | 50 | 50 | 100 | Good | Good | Excellent | Good | Good | Good |
| Example 136 | Sample B | 50 | 50 | 100 | Excellent | Good | Excellent | Good | Good | Good |
| Example 137 | Sample C | 50 | 50 | 100 | Excellent | Good | Excellent | Good | Good | Good |
| Example 138 | Sample D | 50 | 50 | 100 | Excellent | Good | Excellent | Good | Excellent | Good |
| Example 139 | Sample E | 50 | 50 | 100 | Excellent | Excellent | Excellent | Good | Excellent | Excellent |
| Example 140 | Sample F | 50 | 50 | 100 | Excellent | Excellent | Excellent | Good | Excellent | Excellent |
| Example 141 | Sample G | 50 | 50 | 100 | Excellent | Excellent | Good | Good | Good | Excellent |
| Example 142 | Sample H | 50 | 50 | 100 | Excellent | Excellent | Good | Good | Good | Excellent |
| Comp. Ex. 12 | — | — | 100 | 100 | Failed | Failed | Excellent | Good | Excellent | Fair |

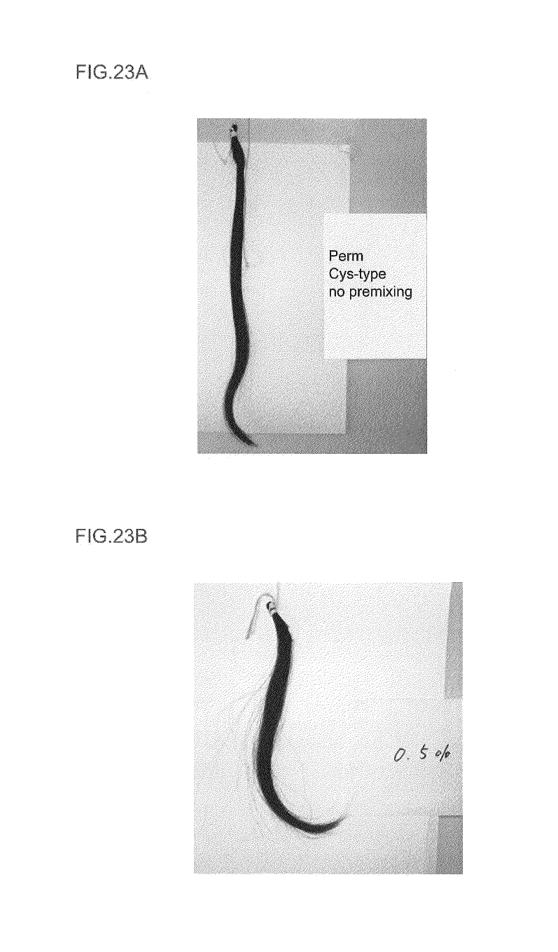

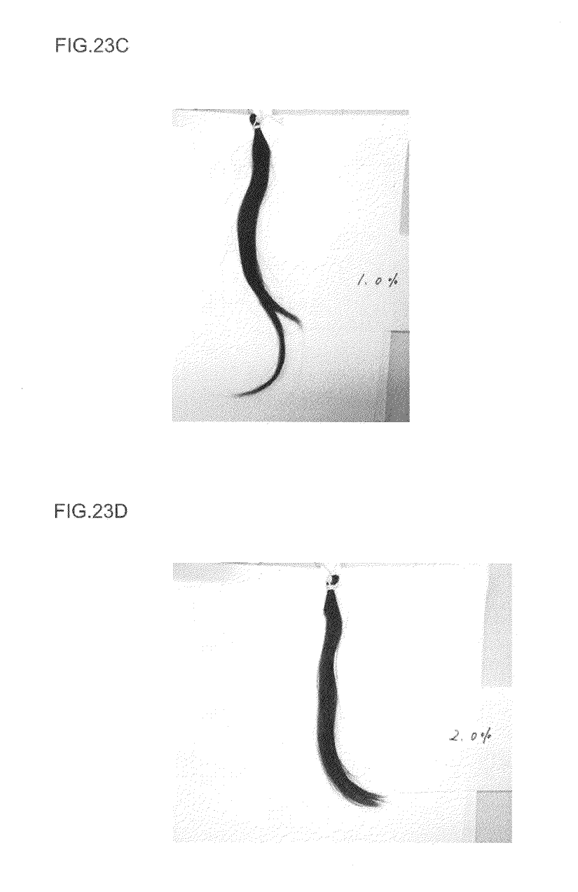

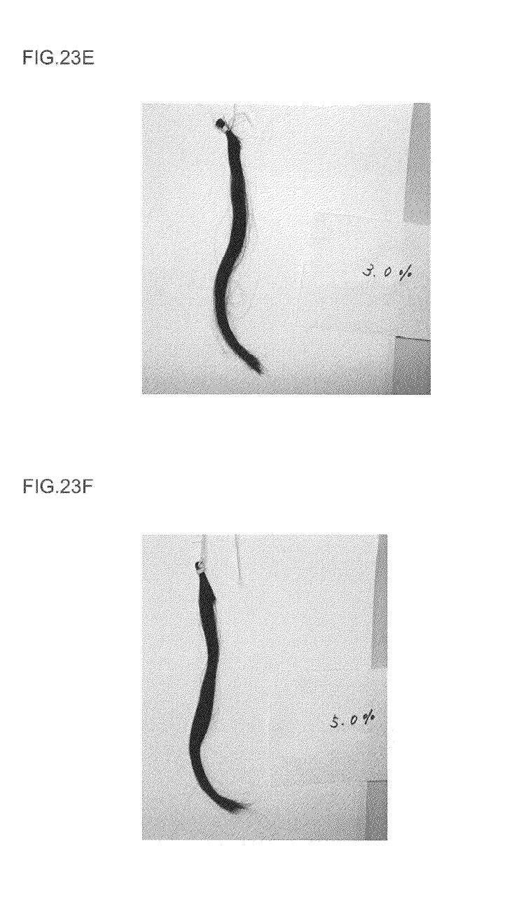

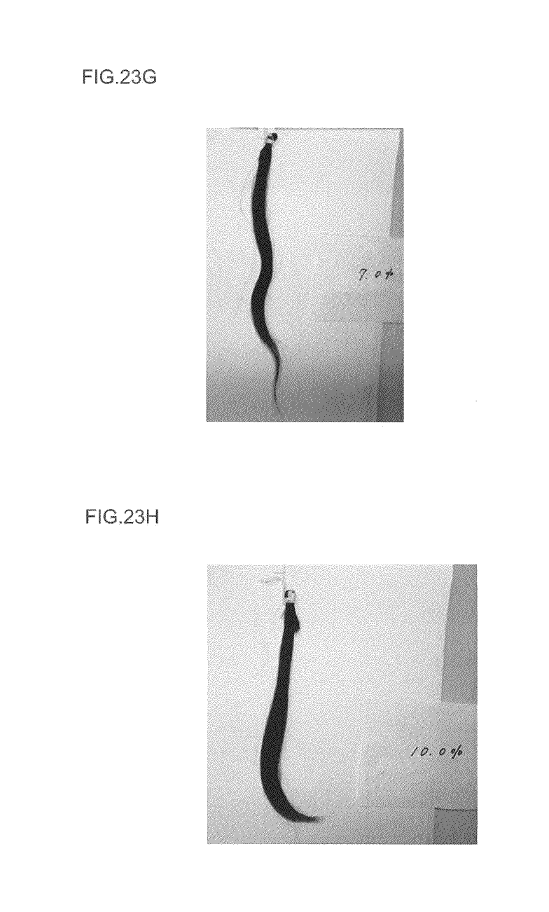

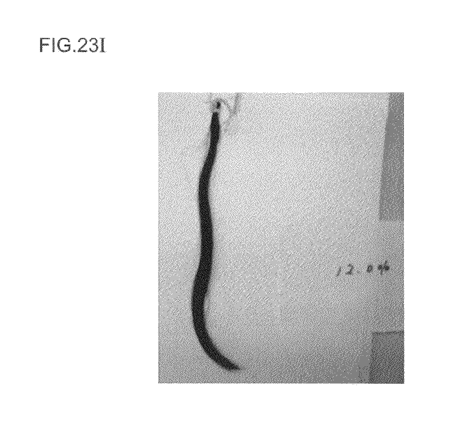

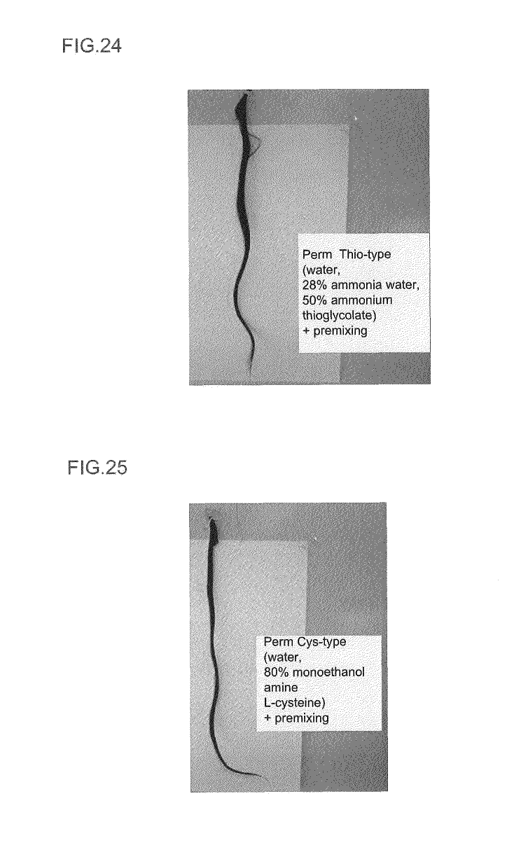

Photos of bundles of hair subjected to the processing with the hair processing agent compositions for perming of Comparative Example 12 and Examples 135 to 142 are shown in FIGS. 23A to 23I.

### Examples 143 and 144

A thio-based perm solution (first agent) and a cys-based perm solution (first agent) were prepared by blending the aforementioned materials to have compositions shown in Table 26. A bromic acid-base second agent was prepared by blending the aforementioned materials to have the compositions shown in Table 27. Note that the combination of the “thio-based perm solution (first agent)” shown in Table 26 with the “bromic acid-based second agent” shown in Table 27 corresponds to a thio-based perm solution configured with minimum components. In addition, the combination of the “cys-based perm solution (first agent)” shown in Table 26 with the “bromic acid-based second agent” shown in Table 27 corresponds to a cys-based perm solution configured with minimum components. The hair processing agent compositions for perming of Examples 143 and 144 were prepared by blending the aforementioned materials to have the compositions shown in Table 28.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the hair (the bundle of hair) was washed with water, and the second agent was applied to the hair (the bundle of hair) and let stand for 15 minutes. Subsequently, the hair (the bundle of hair) was washed with water and then dried.

Regarding “Perm”, “excellent” was given to the case where the bundle of hair could be curled as in the use of a simple combination of the “thio-based perm solution (first agent)” (Table 26) or the “cys-based perm solution (first agent)” (Table 26) and the “bromic acid-based second agent” (Table 27). “Good” was given to the case where the curling condition was slightly weak in comparison with the curl in the general use of the aforementioned simple combination. “Fair” was given to the case where the curling (curling condition) was weak. The results are shown in Table 28.

TABLE 26

| | Perm solution | | | | | |
|---|---|---|---|---|---|---|
| | | | 50% ammonium | | | |
| | Purified water | 28% ammonia water | thioglycolate solution | 80% monoethanol-amine | L-cysteine | Total |
| First agent | (mass %) | (mass %) | (mass %) | (mass %) | (mass %) | (mass %) |
| Thio-based perm solution | 87.00 | 3.00 | 10.00 | 0.00 | 0.00 | 100.00 |
| Cys-based perm solution | 93.00 | 0.00 | 0.00 | 2.00 | 5.00 | 100.00 |

TABLE 27

| Perm | | | | Dipotas- | |
|---|---|---|---|---|---|
| solution | Purified | Sodium | Monosodium | sium | |
| Second | water | bromate | phosphate | phosphate | Total |
| agent | (mass %) | (mass %) | (mass %) | (mass %) | (mass %) |
| Bromic | 91.9 | 7.00 | 1.00 | 0.1 | 100.0 |
| acid-based | | | | | |
| second | | | | | |
| agent | | | | | |

TABLE 28

| | Hair processing agent composition for perming | | | | | | | | | | |
|---|---|---|---|---|---|---|---|---|---|---|---|
| | | | | | Second agent | | | | | | |
| | First agent | | | | Perm | | | | | | |
| | Hair processing | | | | solution | Performance evaluation | | | | | |
| | agent composition | | Perm solution | | (second | Bounce | | | | | |
| | | Content | (first agent) | | agent) | and | | | | | |
| | Kind | (g) | Kind | (g) | (g) | resilience | Rise | Stiffness | Sustainability | Perm | Voluminousness |
| Example | Sample D | 50 | Thio-based | 50 | 100 | Excellent | Excellent | Good | Good | Excellent | Excellent |
| 143 | | | perm | | | | | | | | |
| | | | solution | | | | | | | | |
| Example | Sample D | 50 | Cys-based | 50 | 100 | Good | Good | Good | Good | Good | Good |
| 144 | | | perm | | | | | | | | |
| | | | solution | | | | | | | | |

Photos of bundles of hair subjected to the hair processing agent compositions for perming of Examples 143 and 144 are shown in Tables 24 and 25.

### Examples 145 to 152, Comparative Example 13

The hair processing agent compositions for perming of Examples 145 to 152 and Comparative Example 13 were prepared by blending the aforementioned materials to have compositions shown in Table 29.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the second agent was applied to the hair (the bundle of hair) and let stand for 15 minutes. Then, the hair (the bundle of hair) was washed with water, and then the second agent was applied to the hair (the bundle of hair) and let stand for 15 minutes. Subsequently, the hair (the bundle of hair) was washed with water and dried.

Regarding “Perm”, “excellent” was given to the case where the bundle of hair could be curled as in the use of a combination of “FELADYCA tocosme Cream L I CREAM LINER <hair treatment>” (first agent) (Number Three Inc.) and “FELADYCA tocosme Second Cream II SECOND CREAM <hair treatment>” (second agent) (Number Three Inc.). In addition, “good” was given to the case where the curling condition was slightly weak in comparison with the curl in the general use of the aforementioned simple combination, and “fair” was given to the case where the curling condition was weak. The results are shown in Table 29.

TABLE 29

| | Hair processing agent composition for perming | | | | | |
|---|---|---|---|---|---|---|
| | | | | Second agent | | |
| | First agent | | | Perm solution | | |
| | | | Perm solution | (second agent) | | |
| | | | (first agent) | (g) | | |
| | | | | FELADYCA | | |
| | | | FELADYCA | tocosme Second | | |
| | | | tocosme Cream | Cream II | | |
| | Hair processing | | L I CREAM | SECOND | Performance | |
| | agent composition | | LINER <hair | CREAM <hair | evaluation | |
| | | Content | treatment> | treatment> | Bounce and | |
| | Kind | (g) | (g) | (g) | resilience | Rise |
| Example 145 | Sample A | 50 | 50 | 100 | Good | Good |
| Example 146 | Sample B | 50 | 50 | 100 | Good | Good |
| Example 147 | Sample C | 50 | 50 | 100 | Excellent | Excellent |
| Example 148 | Sample D | 50 | 50 | 100 | Excellent | Excellent |
| Example 149 | Sample E | 50 | 50 | 100 | Excellent | Excellent |
| Example 150 | Sample F | 50 | 50 | 100 | Excellent | Excellent |
| Example 151 | Sample G | 50 | 50 | 100 | Excellent | Excellent |
| Example 152 | Sample H | 50 | 50 | 100 | Excellent | Excellent |
| Comp. Ex. 13 | — | — | 100 | 100 | Failed | Failed |

| | Performance evaluation | | | |
|---|---|---|---|---|
| | Stiffness | Sustainability | Perm | Voluminousness |
| Example 145 | Excellent | Good | Good | Good |
| Example 146 | Excellent | Good | Good | Good |
| Example 147 | Excellent | Good | Excellent | Excellent |
| Example 148 | Excellent | Good | Excellent | Excellent |
| Example 149 | Excellent | Good | Good | Excellent |
| Example 150 | Excellent | Excellent | Good | Excellent |
| Example 151 | Good | Excellent | Good | Excellent |
| Example 152 | Good | Excellent | Good | Excellent |
| Comp. Ex. 13 | Excellent | Good | Excellent | Good |

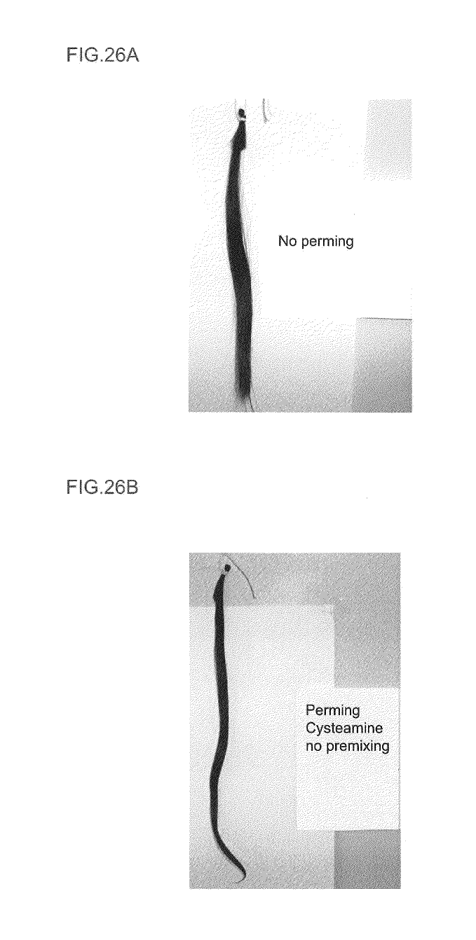

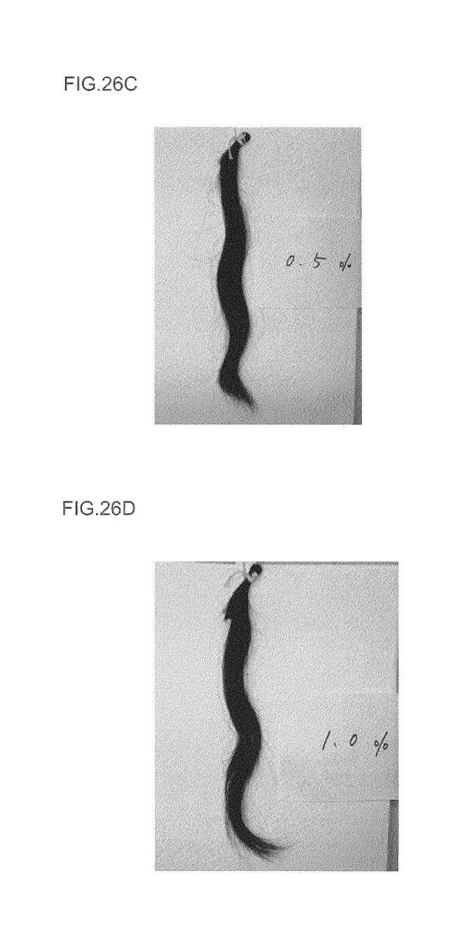

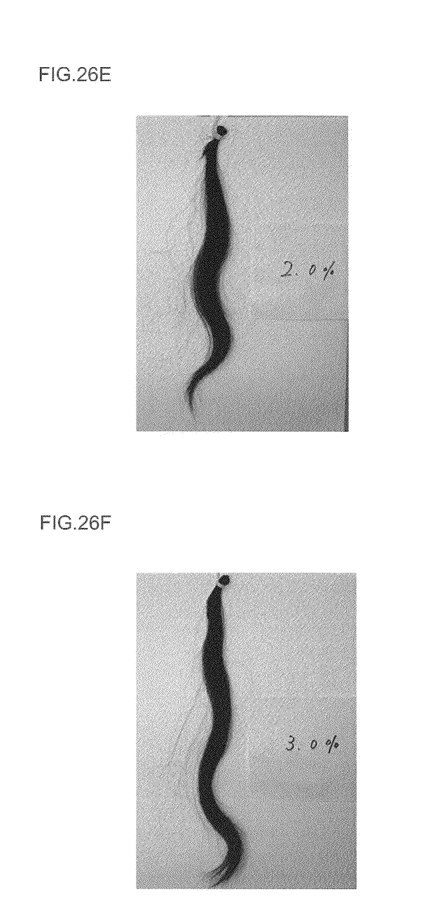

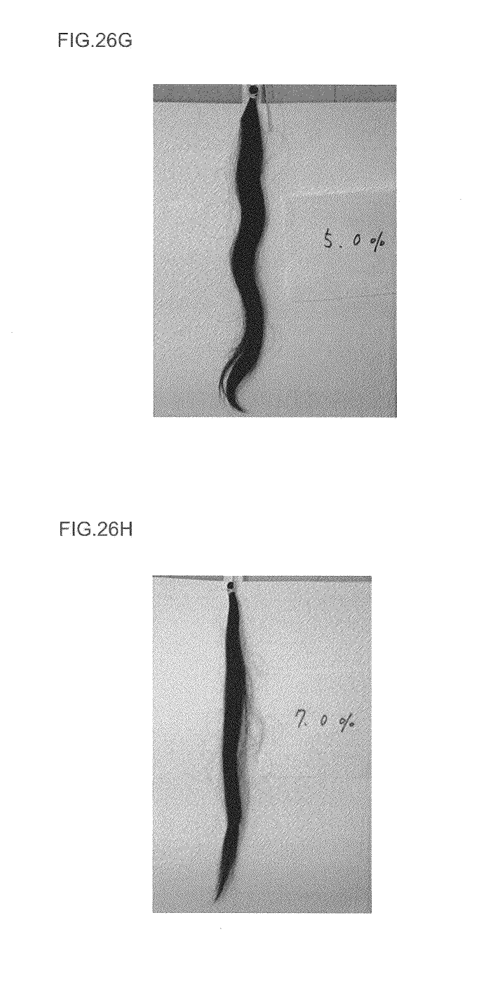

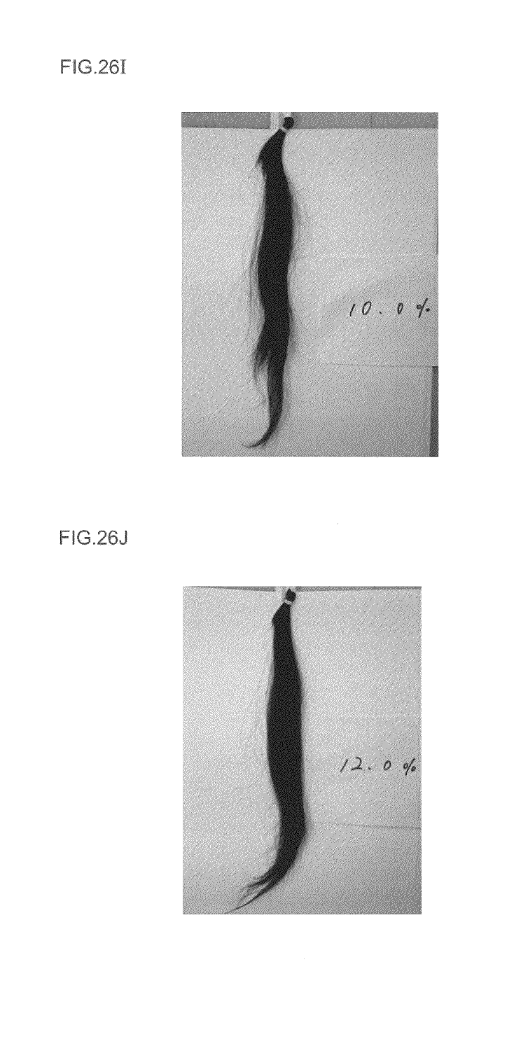

A photo of an unprocessed bundle of hair is shown in FIG. 26A, and photos of bundles of hair subjected to the processing with the hair processing agent compositions for perming of Comparative Example 13 and Examples 145 to 152 are shown in FIGS. 26B to 26J.

### Examples 153 to 160, Comparative Example 14

The hair processing agent compositions for perming of Examples 153 to 160 and Comparative Example 14 were prepared by blending the aforementioned materials to have compositions shown in Table 30.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the hair (the bundle of hair) was washed with water, and the second agent was applied to the hair (the bundle of hair) and let stand for 15 minutes. Subsequently, the hair (the bundle of hair) was washed with water and then dried.

Regarding “Perm”, “excellent” was given to the case where the bundle of hair could be curled as in the general use of a combination of “Safety Wave Labo SP Curl 1, Spiera R-mixed two-bath type curling agent cosmetic product, exclusive to professionals” (first agent) (Nousfit Corporation) and “Safety Wave Labo SP Fix 2, cosmetic product exclusive to Safety Wave Labo SP, exclusive to professionals for fixing curl” (second agent) (Nousfit Corporation). In addition, “good” was given to the case where the curling condition was slightly weak in comparison with the curl in the general use of the aforementioned combination, and “fair” was given to the case where the curling condition was weak. The results are shown in Table 30.

TABLE 30

| | Hair processing agent composition for perming | | | | | | | | | |
|---|---|---|---|---|---|---|---|---|---|---|
| | | | | Second agent | | | | | | |
| | First agent | | | Perm solution | | | | | | |
| | | | Perm solution | (second agent) | | | | | | |
| | | | (first agent) | Safety Wave | | | | | | |
| | | | Safety Wave | Labo SP Fix 2 | | | | | | |
| | | | Labo SP Curl 1 | exclusive to | | | | | | |
| | | | Spiera-mixed | Safety Wave | | | | | | |
| | Hair processing | | two-bath type | Labo SP | | | | | | |
| | agent composition | | acidic curling | for fixing curl [symbol] | Performance evaluation | | | | | |
| | | Content | agent | (second agent) | Bounce and | | | Sustain- | | Voluminous- |
| | Kind | (g) | (g) | (g) | resilience | Rise | Stiffness | ability | Perm | ness |
| Example 153 | Sample A | 50 | 50 | 100 | Good | Good | Excellent | Good | Good | Good |
| Example 154 | Sample B | 50 | 50 | 100 | Good | Good | Excellent | Good | Good | Good |
| Example 155 | Sample C | 50 | 50 | 100 | Good | Excellent | Excellent | Good | Excellent | Excellent |
| Example 156 | Sample D | 50 | 50 | 100 | Excellent | Excellent | Excellent | Good | Excellent | Excellent |
| Example 157 | Sample E | 50 | 50 | 100 | Excellent | Excellent | Excellent | Good | Good | Excellent |
| Example 158 | Sample F | 50 | 50 | 100 | Excellent | Excellent | Excellent | Good | Good | Excellent |
| Example 159 | Sample G | 50 | 50 | 100 | Excellent | Excellent | Excellent | Good | Fair | Excellent |
| Example 160 | Sample H | 50 | 50 | 100 | Excellent | Excellent | Excellent | Good | Fair | Excellent |
| Comp. Ex. 14 | — | — | 100 | 100 | Failed | Failed | Excellent | Good | Excellent | Fair |

[symbol]

indicates data missing or illegible when filed

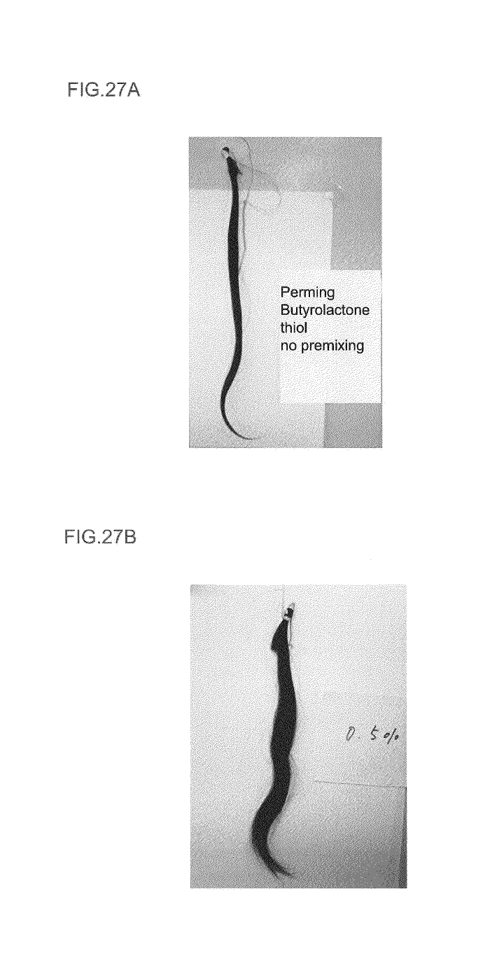

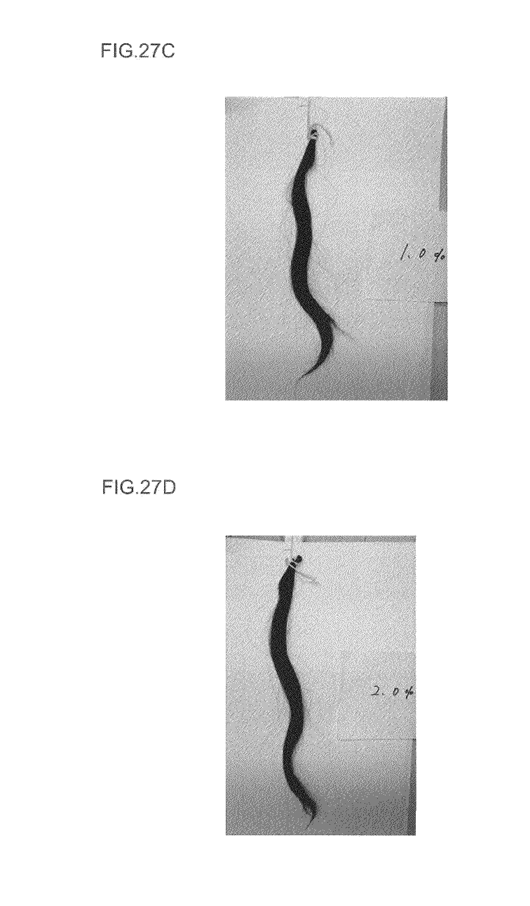

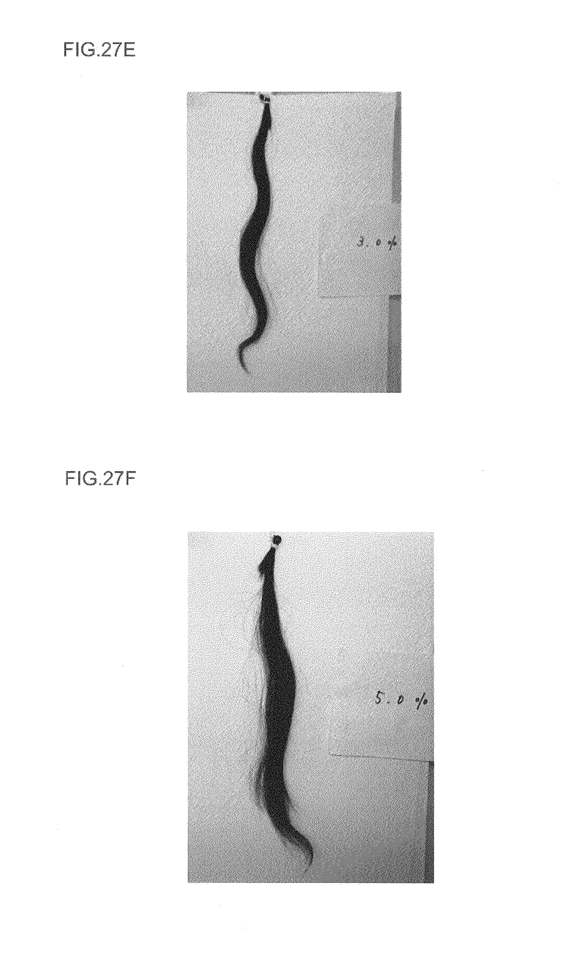

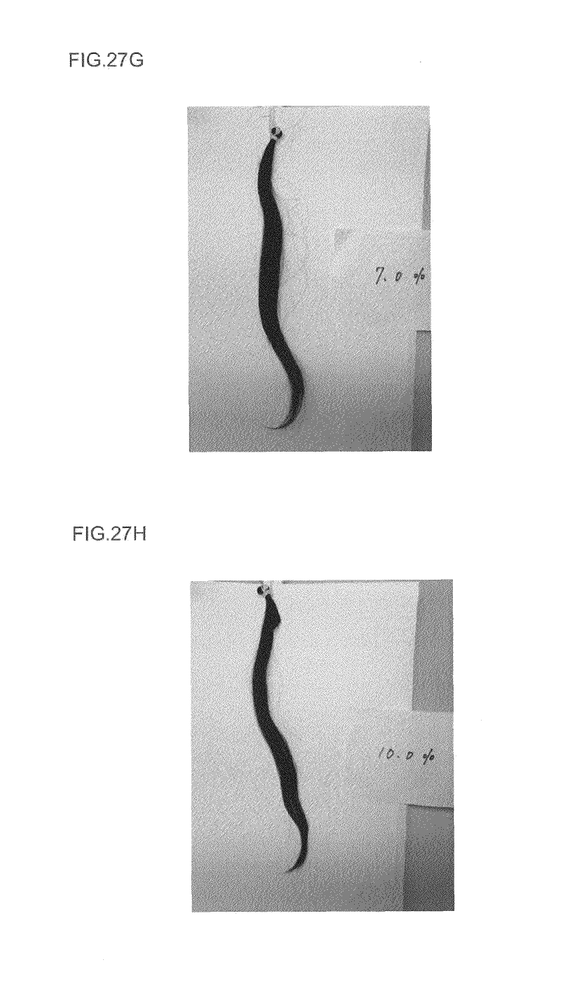

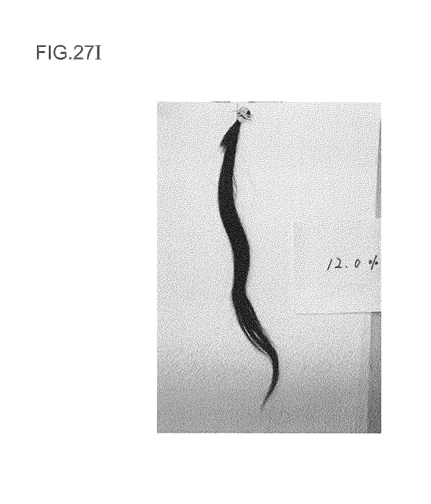

Photos of bundles of hair subjected to the treatment with the hair processing agent compositions for perming of Comparative Example 14 and Examples 153 to 160 are shown in FIGS. 27A to 27I.

### Examples 161 to 168, Comparative Example 15

The hair processing agent compositions for perming of Examples 161 to 168 and Comparative Example 15 were prepared by blending the aforementioned materials to have compositions shown in Table 31.

(Perming)

Hair (a bundle of hair) was washed with a soap-based shampoo, rinsed with water, and dried with a towel. Next, the first agent was evenly applied to the hair (the bundle of hair), and the hair (the bundle of hair) was wound on rods (diameter of 32 mm) and let stand for 15 minutes. Then, the hair (the bundle of hair) was washed with water, and the second agent was applied to the hair (the bundle of hair) and let stand for 15 minutes. Subsequently, the hair (the bundle of hair) was washed with water and then dried.

Regarding “Perm”, “excellent” was given to the case where the bundle of hair could be curled as in the general use of a combination of “Creator Cosme 37<set lotion>” (first agent) (Shiseido Professional) and “Creator Cosme AT <after treatment>” (second agent) (Shiseido Professional). In addition, “good” was given to the case where the curling condition was slightly weak in comparison with the curl in the general use of the aforementioned combination, and “fair” was given to the case where the curling condition was weak. The results are shown in Table 31.

TABLE 31

| | Hair processing agent composition for perming | | | | | | | | | |
|---|---|---|---|---|---|---|---|---|---|---|
| | | | | Second agent | | | | | | |
| | | | | Perm solution | | | | | | |
| | First agent | | | (second agent) | | | | | | |
| | | | Perm solution | (g) | | | | | | |
| | Hair processing | | (first agent) | Creator Cosme | | | | | | |
| | agent composition | | Creator Cosme | AT <after | Performance evaluation | | | | | |
| | | Content | 37 <set lotion> | treatment> | Bounce and | | | | | Voluminous- |
| | Kind | (g) | (g) | (g) | resilience | Rise | Stiffness | Sustainability | Perm | ness |
| Example 161 | Sample A | 50 | 50 | 100 | Excellent | Excellent | Excellent | Good | Excellent | Excellent |
| Example 162 | Sample B | 50 | 50 | 100 | Excellent | Excellent | Excellent | Excellent | Good | Excellent |
| Example 163 | Sample C | 50 | 50 | 100 | Excellent | Excellent | Excellent | Excellent | Good | Excellent |
| Example 164 | Sample D | 50 | 50 | 100 | Excellent | Excellent | Excellent | Excellent | Good | Excellent |
| Example 165 | Sample E | 50 | 50 | 100 | Excellent | Excellent | Excellent | Excellent | Good | Excellent |
| Example 166 | Sample F | 50 | 50 | 100 | Excellent | Excellent | Excellent | Excellent | Good | Excellent |
| Example 167 | Sample G | 50 | 50 | 100 | Excellent | Excellent | Good | Excellent | Good | Excellent |
| Example 168 | Sample H | 50 | 50 | 100 | Excellent | Excellent | Good | Excellent | Good | Excellent |
| Comp. | — | — | 100 | 100 | Failed | Failed | Excellent | Good | Excellent | Fair |
| Ex. 15 | | | | | | | | | | |

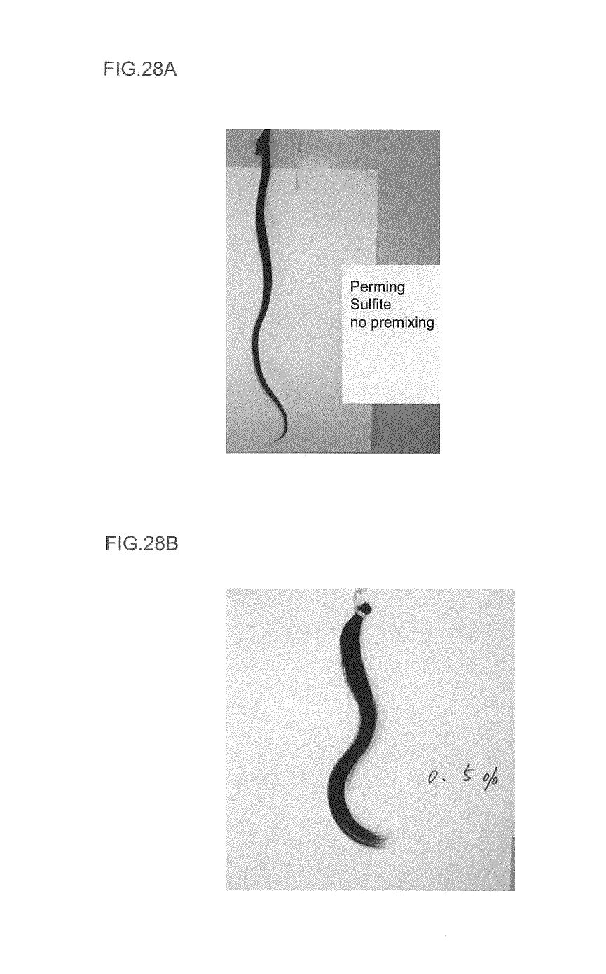

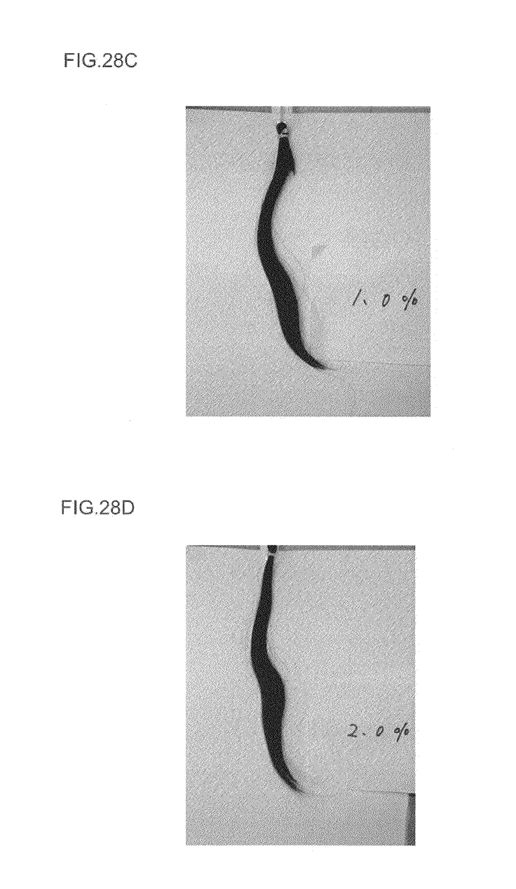

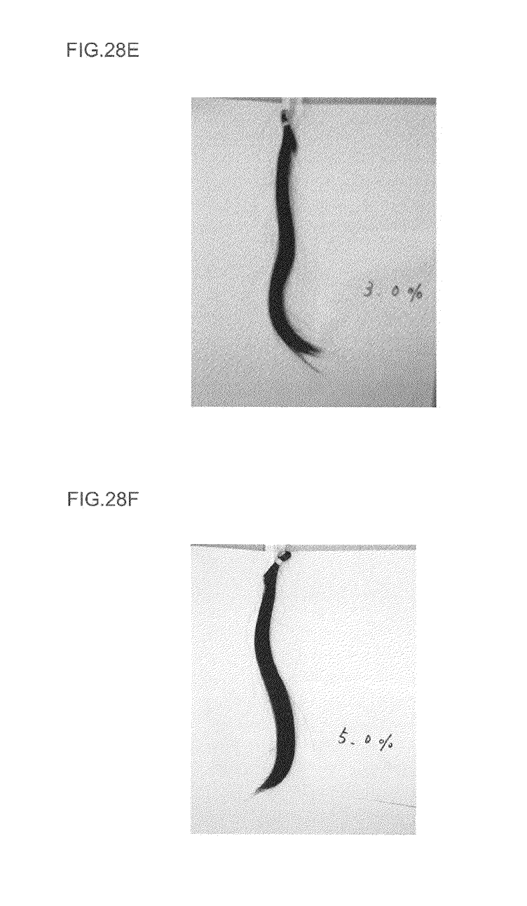

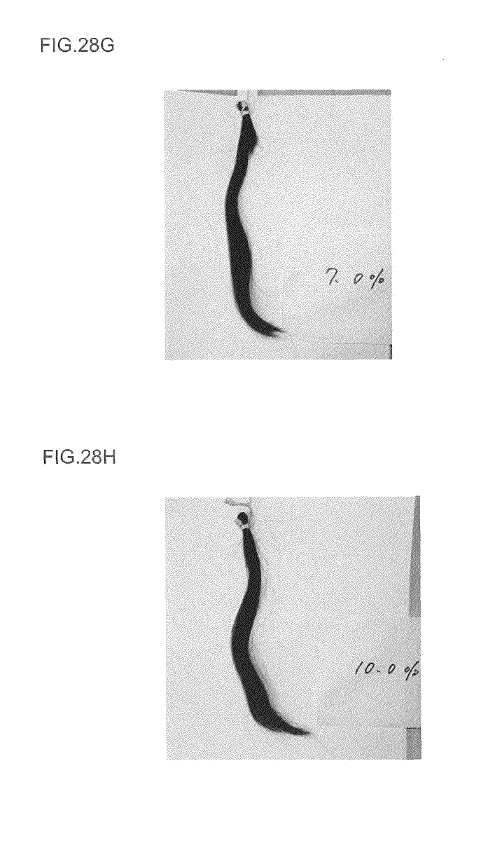

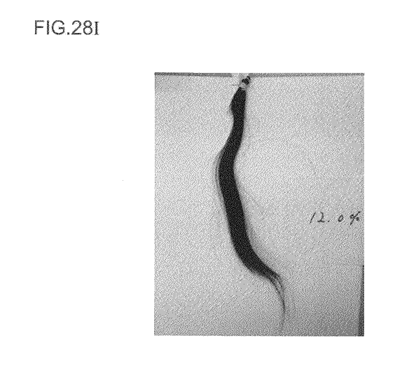

Photos of bundles of hair subjected to the processing with the hair processing agent compositions for perming of Comparative Example 15 and Examples 161 to 168 are shown in FIGS. 28A to 28I.

### INDUSTRIAL APPLICABILITY

The present invention relates to a hair processing agent composition for imparting bounce and resilience to hair, hair processing agent composition for dyeing hair, first agent for oxidation hair dye, second agent for oxidation hair dye, hair processing agent composition for shampooing, hair processing agent composition for hair treatment, hair processing agent composition for perming, thio-based heating two-bath type perm solution first agent, thio-based heating two-bath type perm solution second agent, cys-based heating two-bath type perm solution first agent, and cys-based heating two-bath type perm solution second agent using the aforementioned composition.

### DESCRIPTION OF REFERENCE NUMERALS

1: inorganic particle dispersion, 2: inorganic particle, 3: primary particle (of inorganic particle), 4: secondary particle (of inorganic particle), 5: oil-soluble dispersant, 6: core-shell particle, 7: core portion, 8: shell layer, 9: surfactant, 10: aqueous solvent, 11: first phase, 12: second phase

1. A hair processing agent composition including:
22% by mass or less of inorganic particles having an average particle size of primary particles of 1 nm to 1 μm and
an oil-soluble dispersant.

2. The hair processing agent composition according to claim 1, wherein the oil-soluble dispersant includes cetanol and lanoline alcohol, and a content of the cetanol is 0.5 to 5% by mass and a content of the lanoline alcohol is 0.05 to 0.5% by mass with respect to the entire mass of the hair processing agent composition.

3. The hair processing agent composition according to claim 1, including an inorganic particle dispersion where the inorganic particles are dispersed in the oil-soluble dispersant in a state of primary particles and/or secondary particles having an average particle size of 5 μm or less.

4. The hair processing agent composition according to claim 3, including:
a surfactant, and
an aqueous solvent;
wherein a first phase including the inorganic particle dispersion and a second phase including the aqueous solvent form an emulsion.

5. The hair processing agent composition according to claim 1, including core-shell particles each having a core portion including the primary particles of the inorganic particles or the secondary particles having an average particle size of 5 μm or less of the inorganic particles and a shell layer including the oil-soluble dispersant and enclosing the core portion.

6. The hair processing agent composition according to claim 5, including:
a surfactant, and
an aqueous solvent;
wherein the core-shell particles are dispersed in the aqueous solvent to form a colloid.

7. The hair processing agent composition according to claim 1, wherein the inorganic particles are silica particles.

8. The hair processing agent composition according to claim 1, including a moistening auxiliary.

9. A hair processing agent composition for dyeing hair, including:
the hair processing agent composition according to claim 1, and
a hair dye.

10. The hair processing agent composition for dyeing hair according to claim 9, including:
a first agent including an oxidation dye, and
a second agent including the hair processing agent composition and hydrogen peroxide.

11. The hair processing agent composition for dyeing according to claim 9, including:
a first agent including the hair processing agent composition and an oxidation dye, and
a second agent including hydrogen peroxide.

12. A first agent of an oxidation hair dye including:
the hair processing agent composition according to claim 1, and
an oxidation dye.

13. A second agent of an oxidation hair dye including:
the hair processing agent composition according to claim 1, and
hydrogen peroxide.

14. A hair processing agent composition for shampooing, including:
the hair processing agent composition according to claim 1, and
a shampoo agent.

15. A hair processing agent composition for hair treatment, including:
the hair processing agent composition according to claim 1, and
a hair treatment agent.

16. A hair processing agent composition for perming, including:
the hair processing agent composition according to claim 1, and
a perming agent.

17. The hair processing agent composition for perming according to claim 16, including:
a thio-based heating two-bath type perm solution first agent including the hair processing agent composition, and
a thio-based heating two-bath type perm solution second agent.

18. The hair processing agent composition for perming according to claim 16, including:
a thio-based heating two-bath type perm solution first agent, and
a thio-based heating two-bath type perm solution second agent including the hair processing agent composition.

19. The hair processing agent composition for perming according to claim 16, including:
a cys-based heating two-bath type perm solution first agent including the hair processing agent composition, and
a cys-based heating two-bath type perm solution second agent.

20. The hair processing agent composition for perming according to claim 16, including:
a cys-based heating two bath type perm solution first agent, and
a cys-based heating two-bath type perm solution second agent including the hair processing agent composition.

21. A thio-type heating two-bath type perm solution first agent including:
the hair processing agent composition according to claim 1, and
a reducing agent.

22. A thio-based heating two-bath type perm solution second agent including:
the hair processing agent composition according to claim 1, and
an oxidizing agent.

23. A cys-based heating two-bath type perm solution first agent including:
the hair processing agent composition according to claim 1, and
a reducing agent.

24. A cys-based heating two-bath type perm solution second agent including:
the hair processing agent composition according to claim 1, and
an oxidizing agent.
